SEQUENCE LISTING (148 sequences)

SEQ ID NO: 1 - DNA, 201 bp, Pisum sativum
  source 1..201: mol_type = genomic DNA
  variation 101: note = g or a
ataaccaattaaaaatgagatttyatttgctgcaaatgttatgactataaaccacttaaaataagaaatgatggtgtatgctctacttcttaaagaacttnttgatttggtttgacttgtcttgatgcartaaccaagataagttgcaattaatggaaaaaaaayaatgaacatacttcataartagtgyttgtaaaycat

SEQ ID NO: 2 - DNA, 201 bp, Pisum sativum
  source 1..201: mol_type = genomic DNA
  variation 101: note = T or C
ccctcctcgggaagatcagcatcgcaaaagctcaaattgttacaagcagttatgtttgcaacaatgctatcaaactgttctattgtgacatcgtgatccangtatgacacgtccaacaccttttgcaaagcttcatggtgtggctcggaattcatcagcaaagataggacaratatctttgacggagtttgaagtagctga

SEQ ID NO: 3 - DNA, 201 bp, Pisum sativum
  source 1..201: mol_type = genomic DNA
  variation 101: note = c or t
tcactgaggatacaaagaagatgtattcargagyaatgaaagaagatgatcaaayagagaactcaactctgatggggatcatctmtggaataagagtaaanaaaggtaagaccttaaccatagctggggataggcacagtcaaagcttccaactgyaggagataacactctgcataggagamgtcttccaggagcaaacaa

SEQ ID NO: 4 - DNA, 201 bp, Pisum sativum
  source 1..201: mol_type = genomic DNA
  variation 101: note = g or c
cttgactytgaaaagtcaaagctagttgagaaatttgtagatatggagatcacttactcaggttctgaagggaagaatttagaagccataaaagctgaagntaaagactctgaagatccttagttagaagttgttgaagctcaaactcctttgcaccagttgccaagttatagtytattcgtytcttaatctcttttgcaa

SEQ ID NO: 5 - DNA, 201 bp, Pisum sativum
  source 1..201: mol_type = genomic DNA
  variation 101: note = c or t
gactctaasatagaagactcattccaattttctaaaaggtttcccagacgctacacttataaactttaggaagcaactgttttgaatcgactacatatcanrtgtgcartctaaggtattgatcctaaccataattcatttaatttatttttcttcattgaagtgataaccttatatgttgtcaccgtaytagaagtccaa

SEQ ID NO: 6 - DNA, 201 bp, Pisum sativum
  source 1..201: mol_type = genomic DNA
  variation 101: note = g or c
gtaayagttttcgaatggaaatcatctatgaatgtaacaaagtatatgttrcatcyaattaaatccacctggataggaccrcatacatyagagtatgtganttcaagaattgccttcgacctgcttcytgcatccttactgaagttattcttgtgctacttcggctgtacacattcttcacacacttcatttggaatgycr

SEQ ID NO: 7 - DNA, 201 bp, Pisum sativum
  source 1..201: mol_type = genomic DNA
  variation 101: note = a or g
cagacaaagaggamtatccagtctttggaggagttgttgagggcttgtgttctagagcatgacrrtacttggatagttacttkcctctgatagagttcayntacaataacaaytaccmttctagtattgggatgacactattcgaggtgttgtatgataggagatgtaggactcctctatgytggtatgattctggagaga

SEQ ID NO: 8 - DNA, 201 bp, Pisum sativum
  source 1..201: mol_type = genomic DNA
  variation 101: note = t or c
atttggmtccarattcgaggtgttcagtgatcaaaagagtttgaagtatttattcaatcaaaaagakttsacwatgaggcagaagagatggctygaattcntraaggattattattttagattgagctaccctcctggtaaarccaatkttgyaactratgcgctawgtaggaagttgttacatatgtcgatgctgatgat

SEQ ID NO: 9 - DNA, 201 bp, Pisum sativum
  source 1..201: mol_type = genomic DNA
  variation 101: note = g or a
ttggmtccarattcgaggtgttcagtgatcaaaagagtttgaagtatttattcaatcaaaaagakttsacwatgaggcagaagagatggctygaattcytnaaggattattattttagattgagctaccctcctggtaaarccaatkttgyaactratgcgctawgtaggaagttgttacatatgtcgatgctgatgattt

SEQ ID NO: 10 - DNA, 201 bp, Pisum sativum
  source 1..201: mol_type = genomic DNA
  variation 101: note = t or c
aggtattctccctttccagatttcatataaagtagtaggagtccctttcttcaaggttactcttgaacatgttcatagcttcagcccaaaagtggtagggnaatttcttagcatgaatcatagctctggctgagtcttggagagttctatttttcctttctatcacaccattttgctgaggagtaatgggagatgagaacc

SEQ ID NO: 11 - DNA, 201 bp, Pisum sativum
  source 1..201: mol_type = genomic DNA
  variation 101: note = g or a
aaygagatttgtttakgrccttaaaarmtttygatgataacaaaatatttaaaraacaaaagagtttgctaacattttgtttaagtgtgcaagatcacaancattgaattaattctgcttgttagaatttgattgcctcygaaggcatcagattctggcatcatgacgcaacttctgatgayaactcaaaytctgaaggtg

SEQ ID NO: 12 - DNA, 201 bp, Pisum sativum
  source 1..201: mol_type = genomic DNA
  variation 101: note = c or t
gagcttcaaggtcctaaaaaaatggtgaatggaggtttcccttttcaaatgtcgatgctcacaaagaacaactatggcaattggagtatcaagatgaaggngctactagaagctcaagatgtgtgagatatcgttaagaaagacttcaaggagcaaaatgaagtctcgctaagccaaggtgtaaaggagacattgaaggag

SEQ ID NO: 13 - DNA, 201 bp, Pisum sativum
  source 1..201: mol_type = genomic DNA
  variation 101: note = a or g
caacattgtatttatattgcttgayraaagcttcgccaagatcattgaaggagmggatgtttgcactatccaaacccatataccatctragagcggcaccngacaggctgtcctggaaatagtggataaggagttgatcgttgtctrtctgagtcammatcttrcgtgcgtacatcacaagatggctgrgagggcaagtgt

SEQ ID NO: 14 - DNA, 201 bp, Pisum sativum
  source 1..201: mol_type = genomic DNA
  variation 101: note = a or g
tttctcacttgtgtatggaatggaagtggtgytgccggttgaggtccagcttccatcgttgagagtcctgctggatgtgaaattgaaagaggctgaatggntaagaactcggtatgaggagttgagcctgattgaggaaaagaggctggcgtccatttgccatgggcagttgtaccaacagcgaatgaagcatgcttttga

SEQ ID NO: 15 - DNA, 201 bp, Pisum sativum
  source 1..201: mol_type = genomic DNA
  variation 101: note = g or a
ccatgcaagcatgcaagcatgcaagagtgattttcrwgttttgccaaaaatggaagtgtgcaaatatcagtcccattggcctatawatataagctctcttnctcakaaataggaacctcatgtgcaagctttgattcarcaaccctaaaccctcaccattaaaggataagcttgaagattttctttgaaaatcgagtttca

SEQ ID NO: 16 - DNA, 201 bp, Pisum sativum
  source 1..201: mol_type = genomic DNA
  variation 101: note = c or t
caaaaggattaaaaamacaaataaaaatcaggcataaaagtgctcaaaaaattaaaatggatacactacaataatctgcgcgaaataaactcgaaacatanaggtttttgtcaaattttgaaatggaaaacgcyaggttaaaatgctcagacggattgaaatgtgccaaaaaatttagccgaaaaataaatcgagcacacy

SEQ ID NO: 17 - DNA, 201 bp, Pisum sativum
  source 1..201: mol_type = genomic DNA
  variation 101: note = g or a
tcattctctctctctctctctctctctctctctctctctttctctctctctctctctccttactaaaagtgtctcacacactaacaaatcctactgtggcnaagactaaaatggatgtggtgagatttcctatcyrtactagttgtcatgttaycattaatgaagttcttaaagttaaaatcaatgggatctycttcagaa

SEQ ID NO: 18 - DNA, 201 bp, Pisum sativum
  source 1..201: mol_type = genomic DNA
  variation 101: note = g or a
gaaatttaaattttatattgcaacttcacataatttattgtatattactattattatcgatttgcttatcaacaacatttgatattttttgctatgatttncttgcttaatattcaaccaaattttgattaattcacgttaggttaaaaactcttggctgtaatatttttcaattaaactaactaactcaatctattgaat

SEQ ID NO: 19 - DNA, 201 bp, Pisum sativum
  source 1..201: mol_type = genomic DNA
  variation 101: note = g or a
ttcactattacttcttcttacatatactccatgytcakttgtgcacttataaattccttctctsttaggaaattgtctatcttcttatttcaagctcwtgnagcytgcttaagtccatayagggctttatgcagcctgcataccttgctttcttmgccatgtttmrtaaatccaactagttgtgcaacataaamttcktca

SEQ ID NO: 20 - DNA, 201 bp, Pisum sativum
  source 1..201: mol_type = genomic DNA
  variation 101: note = a or g
tactgatgaatatttttatatgattgtgagattttattgtgatatggatgagatgtgaatacatgataytcaagatgcagtgtgtgaatctatcatgattntttatgcaccatttattatttgaatgtattctcaccactttttgtattgttgcgtgtgcgctcctatgataacaaataatgagatatttgagtatgaact

SEQ ID NO: 21 - DNA, 201 bp, Pisum sativum
  source 1..201: mol_type = genomic DNA
  variation 101: note = a or g
taaaatcatataccatccaagagytccttctttaaaggtaccaaccatgagtttacatttaagggagtcagaagcccctactatttccatctgattactgntcrtaataatatgctcmtgtrggttggttttatcattaaatgacaccagtgaaggcagtttaaagttttctrggacttgatcatcccagatagcctcrga

SEQ ID NO: 22 - DNA, 201 bp, Pisum sativum
  source 1..201: mol_type = genomic DNA
  variation 101: note = t or c
catctaagctccccatgagctattttctragccsagatgtcsagaagttttggagcgatattacrtgtggaagtagtcctgacccttccacccaagtctcnagmaagattcacaaccctgctttcaggtacttccagatgatcatttcccacaccttcctgaggaagagtgaccctgatatgcatgtgagtgttgaagaga

SEQ ID NO: 23 - DNA, 201 bp, Pisum sativum
  source 1..201: mol_type = genomic DNA
  variation 101: note = c or t
cgrccrttattcttatttctaaaggttttctcgatattttcctattcctttattaggaataaataaagttcggtggcgactctgtttcgaacaatttttcngcgttccatcgcgagggatcgcattatcatcatttttttcgaggtgcgacagaactcactacacagctcttttatcatcttgttgttcaagttagatcca

SEQ ID NO: 24 - DNA, 201 bp, Pisum sativum
  source 1..201: mol_type = genomic DNA
  variation 101: note = t or c
gttgttttccaaaatatgacggtaagacggcttgccccagtattttgaatcttatagaggtatgcccctgattggctaacttttggagtgacgggcttcanagtaccatggtagtgacttgcyccagtatgagccttggagagacctgttgcgactcaaagtgattgccccaaattcactgaattttgaaacaacttacac

SEQ ID NO: 25 - DNA, 201 bp, Pisum sativum
  source 1..201: mol_type = genomic DNA
  variation 101: note = a or g
tcaaagatctcaayccaatttataattcagcaacctaaattggacgttatcaacattgattctagatgrcacccaaayaaagaatgattatgtgttgtttntttatgtgtatgcatagaatgtgggttgaacatgatgagaactctttgaaatctcgatttcatgtagrtttactctagaaccttactagtaatgatgcat

SEQ ID NO: 26 - DNA, 201 bp, Pisum sativum
  source 1..201: mol_type = genomic DNA
  variation 101: note = a or g
tttgaaacccatttctttggcgaattcctgcattttctgaccagtaaatacttatccttgatctgttgtgattgtctatggaattccaaacctatagatgntatgtttttggataaattcgatcacatcctcttggttcacatttggcaagggyatggcttcaatccactttgtaaaataatcgactcccaccaatatata

SEQ ID NO: 27 - DNA, 201 bp, Pisum sativum
  source 1..201: mol_type = genomic DNA
  variation 101: note = g or a
ttttaycctaccmattgtggataatttttrtggatayccactrgggatgggtacarttgccatccctatgagtaggtgaaaggtaaggacaagatarcttnacgtccttgcttgcctgwtgtwttggatgmcattttcgttatgkatataactatgttgagacattttgagaatgatctagttcctagttttattttggat

SEQ ID NO: 28 - DNA, 201 bp, Pisum sativum
  source 1..201: mol_type = genomic DNA
  variation 101: note = g or t
ctagagtggttaggtgacagaggggggttaactyttattttcattttattggttttaacattttctttatttgtgtgtctsrcgytgtggggtgttgattnttgtggtagyatgcacgyggratttatgacarcacccctragtctttatgttgtggcctcratctcttcratcgtaccttggrcktgtcattaccttaca

SEQ ID NO: 29 - DNA, 201 bp, Pisum sativum
  source 1..201: mol_type = genomic DNA
  variation 101: note = a or g
ttttctgttttcccttggaaacaataaaagcacggtgrcgactctggttttattracgttaagcttatccataggttgatggtcatgaatttaccactatngaaattaagtggcgactctgytggggagtagtcctcagtgggtttagcctacttttttatgtgtatatarttgtatatttgatgtgtgtatacttktttt

SEQ ID NO: 30 - DNA, 201 bp, Pisum sativum
  source 1..201: mol_type = genomic DNA
  variation 101: note = c or t
taatgatgagagtcaaataaaaaaacaataaattaagwggaagagaatatcactaataaggatgccatcgagaaagagaaaacttatgtacctycacctcngtataaaccaaaaatatcataccctcaaagactagccaaaaccaagaatgagggtcaatttaaraaattcatytttctcctaaaacaacttcattttact

SEQ ID NO: 31 - DNA, 201 bp, Pisum sativum
  source 1..201: mol_type = genomic DNA
  variation 101: note = a or c
caacaccaagtgycggattcagagaagaagaaggagtccccacaggaygggctgcactatcaacatcatcagctcttctggcrtcaacctggggtttcgrnggcggcgagaaaacacgaccactacgggtcaaaccactgycatcmgaaatactrgtcacagaagtggagggtaaaggcacctccttcccatcttctaatg

SEQ ID NO: 32 - DNA, 201 bp, Pisum sativum
  source 1..201: mol_type = genomic DNA
  variation 101: note = g or a
aggrtaattcagtattttagtattttgtctaygtgkcattattctgtgtatataagtagtagtgtgcttagctattaagtaacataacwgtmtgtacacancagagcartgtgagtgcaaccattttgctgtaaytgtttttagcagagtaarggaaatttgttattctctcttctctctyttcttccatcttcatcttct

SEQ ID NO: 33 - DNA, 201 bp, Pisum sativum
  source 1..201: mol_type = genomic DNA
  variation 101: note = a or g
aaactcatyaaagtgctatgaagcaacctctrgrygtccaccatatattgacctcaaatcccatatctywttcatgycatcaaagtccttactagccgatnatgaatggacaacaayattggaaccttctaaartatttaagccacatattttagaccmtttaatacctcatgttcaactctagtgcaaaakcctagayca

SEQ ID NO: 34 - DNA, 201 bp, Pisum sativum
  source 1..201: mol_type = genomic DNA
  variation 101: note = g or c
ccaatgaaaaaatcaattctgctttcacttttttccatctgaagcaattcatacgttcttttgtgagtttgtaacctcacatctttcacattctcagtgcntcaaaatgatttgtcaagaatttctcatgyttcttttgctaattcaacatcattgaccttttygaaattatctgcatcaacattgatgaatcataaagag

SEQ ID NO: 35 - DNA, 201 bp, Pisum sativum
  source 1..201: mol_type = genomic DNA
  variation 101: note = t or c
agcaaatacaagatgacgttagagcaagttgaggctaaccagattaccatgaggacaaacatcagtacgatccaagagaagatggatcagctgttggaaanaatgctcgcgattgctcaaagagagaggattgaggaggaagaagctagggyaaaaaggagatggagatcgtccccaccaagaagaggctggttcagatac

SEQ ID NO: 36 - DNA, 201 bp, Pisum sativum
  source 1..201: mol_type = genomic DNA
  variation 101: note = t or g
ttgcaacattagggttagcgtagaagaatttggagtaaaggaattggggatgagttgtaaattttaatggataaggatgarcactaggtgccmaaagtctnaaaagttgtsaaggwctgytgytgrtactacttcagtgatgtgacaatgtcatgttgtcttccatgtgcattgtactatcatagtacaayacaaaatatt

SEQ ID NO: 37 - DNA, 201 bp, Pisum sativum
  source 1..201: mol_type = genomic DNA
  variation 101: note = g or a
mctcggtccaatcacaagaccgatmtttctgaaggatcttgaatataggcrcacatgtkccagtcatatgcgatataaatcttgagatatagttcaagcgnccgaggaaacttctgacttgcttctcarttttrggcacatgcatttcttrtattgctttkaccttggcgggatcaacctyaatacccttctcgytgacaa

SEQ ID NO: 38 - DNA, 201 bp, Pisum sativum
  source 1..201: mol_type = genomic DNA
  variation 101: note = g or t
tgtctaagcaatttgcgggcgagagcctttccaccaatatggytgccacatgttcctttgtagacttaggaaagtactaaggcaatttcatattctcccanacatygtaacattggaattactctccttattttgtaaagttttcatacaagaagggtgtatctgggcgcctgcttttggacctttcttgattccacttcc

SEQ ID NO: 39 - DNA, 201 bp, Pisum sativum
  source 1..201: mol_type = genomic DNA
  variation 101: note = g or t
ttgatattgttgggtgttttacctaagtcaygtgctcatttmaaataaactctcttatatggaagagattcrttkacctttgaagaggttcaatcagcctngtactctaagggtttaaacraacgaaaagagcacaagmtatcttttrttgatgaaagattgtccgtaaaaggaaaattcacaaataaagatagtaggttt

SEQ ID NO: 40 - DNA, 201 bp, Pisum sativum
  source 1..201: mol_type = genomic DNA
  variation 101: note = a or t
accgcaaaacaaacaaacactaagtctcccatctgcgagcaaacaagccaatgtttaayyattagaatgtgacataaacattcattcactaaaatcaatynaccaacactcatttgctactaaaaactacgtagctttgagttctccatygcacctrgagatacgtaggagggagattcaacatctcrtcaarcaytataa

SEQ ID NO: 41 - DNA, 201 bp, Pisum sativum
  source 1..201: mol_type = genomic DNA
  variation 101: note = t or c
gatgcagtcattttgaccatggaggaaacttccttgtaccccatttygtcaaagaggagycgtgatgcagataaaaaatctcccataaaagagggactccnaagtgaacgygcctaactagggatgcttcttcaggggagataacttgtttcttatgaaagcctgacatccttcctttccggcwtgcttgcacccttaaaa

SEQ ID NO: 42 - DNA, 201 bp, Pisum sativum
  source 1..201: mol_type = genomic DNA
  variation 101: note = 42
gtttctttcatccacttgatcactggaaacacacggtagctttcccttcttcwmctggctctyaatttgttctmtaacwgaaacaacctcaragaaagttnggaagctggagagtccctwtaaacatatcratcaactatttgtcaaggatatgaggytggacaygagttgcaagttgtatccaccactggrcatattcct

SEQ ID NO: 43 - DNA, 201 bp, Pisum sativum
  source 1..201: mol_type = genomic DNA
  variation 101: note = g or a
ctcytatttataccaattgaaataacckcttacaacggcctacttaccasattgagacaagtggcgcyctgcatgcatgtaactgcccactacgtccmacntatatccttagcagttatataagaacaragaattccmtcccttatttgaattcaaatcctttgtcaaactaacacyagtgacrcttctgtcgaaaacatc

SEQ ID NO: 44 - DNA, 201 bp, Pisum sativum
  source 1..201: mol_type = genomic DNA
  variation 101: note = t or g
caayggcrgtacaatcatgtggatgaaayaacttccacttgagggagacratagtggaaggactcacaytcgagggggagtgttkagatggaaatrtgganacttgagcatttatgagtgatagaayccacccacytatcatcttaagattttgggttgagatgtggtatmttcctctcttgtggtcctgaagcattagtc

SEQ ID NO: 45 - DNA, 201 bp, Pisum sativum
  source 1..201: mol_type = genomic DNA
  variation 101: note = t or c
cacgcactcctgagactaggatttgagatggatatctcgcccatccaagttctcgccattactcaaaacacatcaaaccaatcaatttcttttctagccgncgtgcgattaactcttaaaccttttctcgccgccgtgcgattgacccttcaatccttttctcaaatgaaaggtattttgttttaaaatgatgcaaggcaa

SEQ ID NO: 46 - DNA, 201 bp, Pisum sativum
  source 1..201: mol_type = genomic DNA
  variation 101: note = g or a
ggaaattaagaatatcagcctcaagaagtttcataaccatccaagaataaatacaccaataaaaaggagtgaaaatgaaaaatgatgccagtagaatttangagaaagggttttcaaagaaaggagaaggggaaaaaagaaacctgaacatatcagggcgttgatcgtattcaacatgctgcccattggacgtgtctctcc

SEQ ID NO: 47 - DNA, 201 bp, Pisum sativum
  source 1..201: mol_type = genomic DNA
  variation 101: note = t or c
atgcattggtttattgattgaagaattatgaataggtaatcatatgcaaagtaatcaacttttggacccctttgcttctaattctcaaccataatgacaancaaaatcctacaaagattcagatgacgatagtattcttgattgtcaccctctaacctctgaaacatgatgcaatgggatggatgatgcattgagattgag

SEQ ID NO: 48 - DNA, 201 bp, Pisum sativum
  source 1..201: mol_type = genomic DNA
  variation 101: note = t or c
tccagaakaagagacaatggccaatcatatattgacatgctatgagtaaacatactgagaatgatggatttaaccaactyaccatacctgcaaaatagagnrgcgaacctttccacgaarccaacttgtttataattttatatgcaataaactgcaagaaaagcttmtttggttttcctttaaayaraggaacaccaagat

SEQ ID NO: 49 - DNA, 201 bp, Pisum sativum
  source 1..201: mol_type = genomic DNA
  variation 101: note = t or c
cgtgcccaagtgttttttcacgccgygataactcttttattttaaaatcaaccaacccacaawttttttacaaagaactatgyagctttgaattcttcatngcacctagagatacataggagcaygattcaacatcttgtcaagcatcttaataaaaaataatakttttccctttcctataaacatgtcatagtaattatg

SEQ ID NO: 50 - DNA, 201 bp, Pisum sativum
  source 1..201: mol_type = genomic DNA
  variation 101: note = t or c
gaagytagggctccagtgatgtgttggcgtgcctcyarccatctgatcrtgttaaaacrttttaatctcagyctttgttttrwttgacccagcgtacgcrnrcattkactaaggaccagcgtggatgacatgcacgtggccatcagatatgtcacctcaawtaatgaaggagttctgatggcccttgtttttctattttct

SEQ ID NO: 51 - DNA, 201 bp, Pisum sativum
  source 1..201: mol_type = genomic DNA
  variation 101: note = c or t
ttattgaggtaaattattattccatgataacatgatttaagcttctactcacaatgcattgtgtttctcgtgttatttacacaagggtggaaactatatanggatgacccatgtaaaatttctgtacaaaattggttgtgtcagkagaatctgtgtcctattattggaaaatgggaaaaattcccttatattaccaaaaca

SEQ ID NO: 52 - DNA, 201 bp, Pisum sativum
  source 1..201: mol_type = genomic DNA
  variation 101: note = t or a
ttgtttttgcgagaaataaattggttctaaattcaataacaagaaaaygattaggttttaytttaatcccttgtattgtcccttgttgatgtttaatgganaatatgaatggtaatgtcattatatttaacaaggtaatattaaaagatatccactccacgaggattgcgatttaacaaggtaaaaatmattcgaattaaa

SEQ ID NO: 53 - DNA, 201 bp, Pisum sativum
  source 1..201: mol_type = genomic DNA
  variation 101: note = c or g
agtgaagtgttgtctgaactggtggaagtttcaaaggctcttcaaaagactagtactgctwgtattaccagaaagaggaatataaattagttgatcaaaanattatctaaggagaaggaagttgacaaggcagaagaggytcacaatgaagatgaagagaaagaraagcctgcaaataatggagattctaatagctcaaat

SEQ ID NO: 54 - DNA, 201 bp, Pisum sativum
  source 1..201: mol_type = genomic DNA
  variation 101: note = a or g
cggattccatcagcgtcgtaagtttggcaatctcatccttcagttctctattttcttgctytagatgttccattctctttggttgatcagcagtgttatancggtgagtcagcttkgctgatgcacaaaagaagaactgataagacatctggmgggagaaacctgtcatrcaaatgatgcatgaaatgcaatgttttttta

SEQ ID NO: 55 - DNA, 201 bp, Pisum sativum
  source 1..201: mol_type = genomic DNA
  variation 101: note = g or t
cacgtggcgtctataactayagttattaattctgctccagttgctcaagcarcaccgagttatcaaccgcatttccagcaacacacgaatcaacagaatcntgcccagaggcctgtacaatttgacccaatttcgatgacttatacagagttgtttcctactttaattcagaagaattttgtgcaaacaagaactccaccg

SEQ ID NO: 56 - DNA, 201 bp, Pisum sativum
  source 1..201: mol_type = genomic DNA
  variation 101: note = a or c
aaatttgaagtccattggattaaaatcaagcatttcgcaaaggaaagagtaaggcaaggtcacaaatgcacctatggtcaaagattataacaccagcaaanatcaagccatgaacaactttcaaacttatgatcataaaaaactagacattctaaggatcattatgcaaaaaattgggagtgatatgatgcatttctcaat

SEQ ID NO: 57 - DNA, 201 bp, Pisum sativum
  source 1..201: mol_type = genomic DNA
  variation 101: note = g or a
atcrtctttcaggttgtctccttagtgagcacagagaaagtgatacaccacccagatcccctgagagtctggagtttggtcctaatcttctggctcyatantgaggctggatctagcatytgtgttatcaattatatggttgtcatccctcgtgttgattgacctaaaatcccttatagattgataaattggtatgttgtc

SEQ ID NO: 58 - DNA, 201 bp, Pisum sativum
  source 1..201: mol_type = genomic DNA
  variation 101: note = g or a
cagcttcctcagtgrtctatattattaaccacacttgctaagacattgatggtgatattattgaagttattgagttgctcaattcttgattsaaaacccantagttgagattgtaggtcaatccaactaagagatttgaccagaatgtttaattactacggggttatacttagagtaaaaaccatttaaggtttgaattat

SEQ ID NO: 59 - DNA, 201 bp, Pisum sativum
  source 1..201: mol_type = genomic DNA
  variation 101: note = a or g
ccttctcacaatgakccmttgatcaacatcaarttarwttcaaaatctcrccattttcaaatcatttctcacatatgcgaattaaacctcgatcaacatcnattgryttttcttaaatcaraygtaatatcaaattctttttcttaaacaaaygcgaaagaataggacygttgaartccagtattccagtggaaccctcga

SEQ ID NO: 60 - DNA, 201 bp, Pisum sativum
  source 1..201: mol_type = genomic DNA
  variation 101: note = c or t
caattgaattggagactcctcttaaaattgaatggaggcgccaattggattggcgcccctatgtaggatttaagagraggcgccaattcaactggagcctnagtrtaaaatgcctttttttkggtaaatggtatacgtgatagataaatttgatatatgaccaattgatattaataaaaattgtcgtttacacaaagaaac

SEQ ID NO: 61 - DNA, 201 bp, Pisum sativum
  source 1..201: mol_type = genomic DNA
  variation 101: note = g or c
tgaataccgatctcagaaggcaattaatsgtagtatcttagctgaccatttggctcaccaaccgattgaagattaccartcagtgtagtacgattttcctnatgaagagatcttgtacatgaaaatgaaatattatgatgaatcgttgcttgaagaagggccagaacctggttcccgttggggcatggtatttgatggagt

SEQ ID NO: 62 - DNA, 201 bp, Pisum sativum
  source 1..201: mol_type = genomic DNA
  variation 101: note = g or a
caataaaaaatcragaaattattctaggacacttcatgttttttttaaatatcacatattccctcagtttttgaatataaccggggtgaaaacaaaytcgngacatgcttctaatcccatcaaatgcagtttatgtttttatttctgtaaaagtgttgtctttatttttgttttactttttatttataaattaaaggatkt

SEQ ID NO: 63 - DNA, 201 bp, Pisum sativum
  source 1..201: mol_type = genomic DNA
  variation 101: note = t or g
gcarcaagaaaaagtaagtattttgatttttatcgtctycataggrattaatgtgatatcaatgtcgttcaacggtctctacgattttaatcttggrtttntaagatttgaatttaaaggtaagctatgtaaaacggataaataaataaataaagcttcagagagatataaragttttacaagatwgaatttcatctaccg

SEQ ID NO: 64 - DNA, 201 bp, Pisum sativum
  source 1..201: mol_type = genomic DNA
  variation 101: note = c or g
tttattccatctcattttataaatttagaacagcaaataagcaatgtatggaaactattgtaggacctagcattagagttcccaaaactcgatcctgctgntcttgatcttctttcggtaagttaactgccccgtacacattactgtttacgtttcttcgcctaaattactcarctcttttattgatctacttaaagttta

SEQ ID NO: 65 - DNA, 201 bp, Pisum sativum
  source 1..201: mol_type = genomic DNA
  variation 101: note = t or a
tatctgtytaggtargagctagaaagaaaacarycaatcaagactcaacccaatraggacataactcaakgkgagtaattccrtccagaaaatttgctggngtggaagcagaattagmaatcatmcacgasgaaacaaactcagtrgggaaacagagaaaggktaaagtcttttctrcwtaggggctgacactctacagtt

SEQ ID NO: 66 - DNA, 201 bp, Pisum sativum
  source 1..201: mol_type = genomic DNA
  variation 101: note = g or a
tacrgaawagtggcgactcagctagggartagycctcagtggktttagcctatctttgtttatgtgtatatattatgttcactrttgtttattattgtttnttytgtctggtrcttggtgatctctatgtggtgagataagtcttatamccrtacttgagakmtcttattgtaggagggtggtatagtcttgtttggctta

SEQ ID NO: 67 - DNA, 201 bp, Pisum sativum
  source 1..201: mol_type = genomic DNA
  variation 101: note = c or a
attattatcatgtgaggtttgatatataacaggatcttatgctgcttgtcaagaaatttggattagatatgtgaagcaagatatgaatgtcgaagtgaagnaacctttggtgttgcarattaacaacaagtcatctatgaatctcgcaaagaatccaattctacatgggagaagtaagcacatcgagcctaggtttcatgt

SEQ ID NO: 68 - DNA, 201 bp, Pisum sativum
  source 1..201: mol_type = genomic DNA
  variation 101: note = a or g
tagagaagtttgtgaagaaaataacgagattattcagttacaacaacaactacttttagatatcacaaacacaaatgtgatgatacatarcattgcatcangggaaatgatgatcaataatctttatggcactaaagtaccactacaacattttatactttagrcaacrgaagaataggcaacgattgaaaaaacgtcacc

SEQ ID NO: 69 - DNA, 201 bp, Pisum sativum
  source 1..201: mol_type = genomic DNA
  variation 101: note = c or g
ataataaattcttgaagggactttgatacgtgcattaattcaaacaaaacaaaayttctggmagtgccaracattagagggacttyagggrggtcmtcttnattttttmgataaaaatggatgttggacttagacatgtaggraacatgtatccaagaaaatatggggtagttgaaagttagtggacttrcactaacaccc

SEQ ID NO: 70 - DNA, 201 bp, Pisum sativum
  source 1..201: mol_type = genomic DNA
  variation 101: note = t or c
ctgwgtartgggataattgagaaaggctttgagrcaagggtargaggtgtacrcaytatccttcyggaryctcccctattcyaactcaagtgcactrcctncrwtttaaacgccttttgaccrgatctttcrcccaacttccatttcctagtccataacgtgcatttrtgagtataacgtgtaataagtcaacaaatagtg

SEQ ID NO: 71 - DNA, 201 bp, Pisum sativum
  source 1..201: mol_type = genomic DNA
  variation 101: note = g or a
attgcaracatcatgacgaaagaagtgcatgtcaaagtgtymaagaagttaagatctatgatgaacgtagatagcttagacayaatraattaggtggtgtnttgagaatgtaattcggygtgwcgatgcagcaatttcgacacartcaaggaaatgcatgrtgtgtcaaaktrtaatagttgttgaaagaagtgtgtctty

SEQ ID NO: 72 - DNA, 201 bp, Pisum sativum
  source 1..201: mol_type = genomic DNA
  variation 101: note = t or c
gatgctgagcagttgcgttcatcrccaaatgaggacgactctgattgggaggatgaagaccctgatgacgaaggtatcatttatgtggaatagctgtcccngaaagcatcagtgttggtaagtatgttttgatgagcttatgtcatgaagatgattagttcagttatactttgtatatttgagtttggttygctttagaat

SEQ ID NO: 73 - DNA, 201 bp, Pisum sativum
  source 1..201: mol_type = genomic DNA
  variation 101: note = c or a
gatttaaagacaataatctttattctcagttggtcggcaacatatctcgaggggatttaaattatatttttcacgaagctwaacgatctgataatgtaggntccaatagcacaaagygtggatgcacaattgtgaaaatatatggtmttccatgtgcctgtgttattgcaaaaaasgtgaaacttggtagcctgataagaa

SEQ ID NO: 74 - DNA, 201 bp, Pisum sativum
  source 1..201: mol_type = genomic DNA
  variation 101: note = g or a
aaaycaactayaacctgmcaagyttggatactcagagaaamgaaaggggaaaaaacataccatgataggctactaatttcttcttctcttctgagacaacngcgtttttcttttctggtccagcaatgatcctttcaagtgcatcagatatctcatctttacttatttccttgagctcgcgcctggctgcaagaatagcag

SEQ ID NO: 75 - DNA, 201 bp, Pisum sativum
  source 1..201: mol_type = genomic DNA
  variation 101: note = a or g
tcatgaagatcaccatgtagaaatacatcatggacatctagccgatgaataggtcgygatttggacagagcaatggtgagaayggagygaatagtggccgntttgaccacatgaccraatgtctcattataatctacacctgaaatatgtgaccttccatcacctacaagaygagctttgtaggctgaaagcaaccattag

SEQ ID NO: 76 - DNA, 201 bp, Pisum sativum
  source 1..201: mol_type = genomic DNA
  variation 101: note = c or t
cctcttggtagttaaatacatgtgtcaaarttaactgaaygtaggactgaaagtcatcaaggagaaggggtgggttaaagttaaattttgagccttatatncttttgttcaaaaaaagcatgaaccrgaccatgttacaaacttaaaagacctaattaagrcatgatttattttgaaagcatattgttgaagcgataaagc

SEQ ID NO: 77 - DNA, 201 bp, Pisum sativum
  source 1..201: mol_type = genomic DNA
  variation 101: note = g or a
gtgcatgragaaaacraggaagaaggatcagaaggcgttgtttaatwttcataagcgtgtraatacaaatgtgtttgagaatatcaytaattcaacgacgncgaagrtgaaggtgtgaaaaacayaagaaatgggggtttaaattgggttttataagcaaaarytttttcyaaaacaagaacacyacaaacaagttaaaaa

SEQ ID NO: 78 - DNA, 201 bp, Pisum sativum
  source 1..201: mol_type = genomic DNA
  variation 101: note = c or t
gtwgaacctctgaccttagrgtcatggggtcaacccccttaccaactgagccaagcgctcttggtgacaaaagcgtgcttcaatgaaataaataataaaanaacaacaaaaataaarcasrcgtgtgattcaggcaatsagagtgagacaacaaaagttttcaaatkcaawcgcgtgaargtgacttcgtcttcaaccttt

SEQ ID NO: 79 - DNA, 201 bp, Pisum sativum
  source 1..201: mol_type = genomic DNA
  variation 101: note = a or c
tagcttggttaccactttggttttggttgtttctccaacgaaaattcggatgmtccttccatctcgaattctaggtattggaataagggttatttttctgnaaatttgcaaattgggyctcttcacttaccccttccaacakacacctttcrttttcatgtcctcctccataaaagtcacactaagtgattgtaccyggct

SEQ ID NO: 80 - DNA, 200 bp, Pisum sativum
  source 1..200: mol_type = genomic DNA
  variation 101: note = a or g
caaacacaatatacacaatcacaatatatcacaaaatatggtccaaatggacaaagggaaaatgacattaacataaacaacttgaatggtatgaataatncaaataaataaagcttaaaaattaaagtgcattaaaagtaaaggcttgaaattaaatgttagttgttagttgattagaaattagtattgcttttgattta

SEQ ID NO: 81 - DNA, 201 bp, Pisum sativum
  source 1..201: mol_type = genomic DNA
  variation 101: note = a or g
actgtacaaayaatrtggttggatatgaagcttatgtcataggtctcraagctrctatygacctaaggatcaaaactctggaggtgtacagagactcaacnctygttatctgtcaagtaaaaggmgaatgggtaactcrtcacccaaatttgattccttatcatgatcatgtcttagaattgatgaagaatttygaagaaa

SEQ ID NO: 82 - DNA, 201 bp, Pisum sativum
  source 1..201: mol_type = genomic DNA
  variation 101: note = g or a
ccttctrgatcagctrttrttrccatcaatgcaacattrgctygttcctcgtcagactcwtcttcttcttctgattytgagtcgtcccatgtagccmtcanccccttattrcctttagaaaaatwcttcttaggccytctgtcmttttttagcttaggacaatcatttttatagtggcctkrctccttgyactcaaagcaa

SEQ ID NO: 83 - DNA, 201 bp, Pisum sativum
  source 1..201: mol_type = genomic DNA
  variation 101: note = g or a
ttgrgactgacactaaaaaaatgcaaaattaccttttgttgctatagagcaagaagrggttggagactcgaggagtytgtacakyctgtctagttgctccntagtgagtgaaaactgagacrratgrgattrwtgtccttgatcagaattaccagcctaaaatgcacgrccayttttcttcttmcakttakgaggtttgcc

SEQ ID NO: 84 - DNA, 201 bp, Pisum sativum
  source 1..201: mol_type = genomic DNA
  variation 101: note = a or g
ggtagggatacgcaattytgtttcttgctgaaccatgaaatcaaattgttcccaagaagaaacatcctccrggggtactcttcctatcatyaacactaccngyccaatcracatcataataccccactaatatagaatttgaatcacgtgaatacaacattccatactcacttgtgccattsacatactttagartccttt

SEQ ID NO: 85 - DNA, 201 bp, Pisum sativum
  source 1..201: mol_type = genomic DNA
  variation 101: note = t or c
gtcttcttatctttcaaagatgccccasacggataaatctgactttgggraaaacacttgatatcaaaataccatggcttttcatctttgatctcwtcaanaacaaacacatgagctgcctatcaagacgcatcacagtcaaattgrraacttcattcyagaacttcaccataattattgaaaccaacgttgcaagagcat

SEQ ID NO: 86 - DNA, 201 bp, Pisum sativum
  source 1..201: mol_type = genomic DNA
  variation 101: note = t or c
ttcaaggatccatgcttgtgaatagaggattgagtgttctccaaagaatgacttaaacaattaaaaagcaaaatcccactaacttctaattaaataacatntgactaacttttaatttcaagccatttactcttatgcactttaaattcaaaccttttatcattttccattatacatatcatttgacttgttgatgtttat

SEQ ID NO: 87 - DNA, 201 bp, Pisum sativum
  source 1..201: mol_type = genomic DNA
  variation 101: note = a or g
aggaccaraatcagctaacgtctataaayytctaggatgcaattataattgttccctaaaatcaaccaacaaatcggtattttctatcaagaactacgranctctgattttctcattgtactatgagaatatgtaggcacgaggrttcgaatcctcggagagcacaccaattaaaaacttatttttccccttttatcattg

SEQ ID NO: 88 - DNA, 201 bp, Pisum sativum
  source 1..201: mol_type = genomic DNA
  variation 101: note = c or t
catcmaaaatgcaacttacaygaccwtacstaagcaagattcaacatcgtataacrtacmacaccctaaaataccaatgcatgcagtcatatggaaacatntaacaacascgaacccacttcgttaagaatgaaaaactaaaccaaacacaattatattactcttaacaagacataatgcacgtrcaaaaygagttatctg

SEQ ID NO: 89 - DNA, 201 bp, Pisum sativum
  source 1..201: mol_type = genomic DNA
  variation 101: note = g or t
ctgawgttagtttgtccatcaacttacaaaaaaggttaatggtgcattatggttccaagaaagcatggagtkattwagggagagtaggagtgyttttccknagtttaagggccttgcttmtggtgtttagatagaktaagatcaaascaaaatttatttacaaagctccaagcaaaagtggttgagaaaagtgatctttat

SEQ ID NO: 90 - DNA, 201 bp, Pisum sativum
  source 1..201: mol_type = genomic DNA
  variation 101: note = t or c
ccccaatcttawtttttyctccgwggcccatgctgaaaaatatctcaaactagtggattaccatattgtgagggaaagagaattcactttggataatttanaaggttttrgagaagtgggagaatttttgcaataaaggtgttgggtaagtttaaacaatctaattcatgacwyaaataagagtatcagactagaatttta

SEQ ID NO: 91 - DNA, 201 bp, Pisum sativum
  source 1..201: mol_type = genomic DNA
  variation 101: note = c or t
araaatgaatccactrgggactctactgatggggaaaacaggggtacttttrccgggkattgggcaagaagtaacaaactgcaaactaagaagaatattancagttactsggtaataaactcttaggagacttagagyatckatctaggcaagagctagaaagaaacratyaatcaagactcaacccaatgaggatataac

SEQ ID NO: 92 - DNA, 201 bp, Pisum sativum
  source 1..201: mol_type = genomic DNA
  variation 101: note = c or t
raagatgcaaaagtygaatmatccatcctaaatctcttgagtatctcattgacatactttctttgatgtagcatcataccttgcttygacatttraaattncaygcgtaagaaataaracaagtttccaaaatccgacatatcaaattccctcatcrccagctctttgaacttcgwcaagttcttcragctatttccagtt

SEQ ID NO: 93 - DNA, 201 bp, Pisum sativum
  source 1..201: mol_type = genomic DNA
  variation 101: note = c or a
aggatcccctagcacaaaagcacacacacatcaaagagtcttatgaagcaaatcaagaatggacaagagttagtttagagatttggtccttctaatccatnttcaacattaggcattttactccaattttgcatagggaatatcctagaaactaagtccatttgtccatttmtttgcatttggtccataacaatcaaaaca

SEQ ID NO: 94 - DNA, 201 bp, Pisum sativum
  source 1..201: mol_type = genomic DNA
  variation 101: note = a or g
rtagcatcaagtacaccactcytactrctaccrttttactgtctactcatgatgatgattctatggttaakcwagggggaaaacaaaaaaaatagggttynggagttgttrcggaggagactgraacttttgtagatgagagtgtgtatgcaaatctttccatgtgatcawcgaagagatatttcccacycaggaggcagt

SEQ ID NO: 95 - DNA, 201 bp, Pisum sativum
  source 1..201: mol_type = genomic DNA
  variation 101: note = g or a
agtaacgtcattcgtccaaaacccaatccataaccccgtagttagccgaactatgacttgctctgattctcattccacatgagatacgtaggcataagacntgatgtcttagcgagcatacatccccccaacccataagtcagccgagctacgaagactttgattctcatattcatatgagatacgtatgcagtggatgcg

SEQ ID NO: 96 - DNA, 201 bp, Pisum sativum
  source 1..201: mol_type = genomic DNA
  variation 101: note = a or g
cacatgagttggcctatcaagaygcatcacagtcaaattaggaacctcattccaatacttcactacaatcattgatgccactrttgcgrragcatctgccntccggttttcatctcgagggakatgatgaaattcaacctttgtaaagaaagttgaaatcctcctcgcataatctctatatggtatcaaaccgggttgatt

SEQ ID NO: 97 - DNA, 201 bp, Pisum sativum
  source 1..201: mol_type = genomic DNA
  variation 101: note = t or g
aaatcaagtgaarcaattttttgaacacttagtgtgagattaataatgtttgggattctatgtggtattgttgttctaaaaatccaatcacarccaaatgntttatgatctacacaacaaygctaatttcaaaatgtctttataattatgatccaaacaaaattaatcaaacaatcaatgcaattgacaatttgcaaacca

SEQ ID NO: 98 - DNA, 201 bp, Pisum sativum
  source 1..201: mol_type = genomic DNA
  variation 101: note = t or c
ccagtaactrgtatataaatyccacttctcccctcaragttaatmtttgatatgttcatcctaaccgwtgacgrattttctccctttatggttttctaccnartaaaaaggtaattataaatcctgttttcccctgctgagtctatcctttatatgttcatcctaactgatgacaaatattctctttttggtattctatcc

SEQ ID NO: 99 - DNA, 201 bp, Pisum sativum
  source 1..201: mol_type = genomic DNA
  variation 101: note = a or g
gtccaggccatctgatccttggtccacattttaatttgagtcatacaatgcaattaccatgtgtgcaagcgcgttgactgatgaccactgtggatgatacncgcttggccatcatatttgccacctcaattaakgagggagatcttatggccctcatttttttgaattgaccgactttctattttaatattttaattagca

SEQ ID NO: 100 - DNA, 201 bp, Pisum sativum
  source 1..201: mol_type = genomic DNA
  variation 101: note = a or t
tcatcctacaacaagaaatcagatgggagcaatcaattcaattatyatyrttagaagcaaagtattgtgacctcgaagacatagggataataacatgtgtnrggttcaatagggacatgaataaatatgwttttcattgccatttttwgattttcggtaatttccccttacgggaattacttctttatgtaatatcgcaca

SEQ ID NO: 101 - DNA, 201 bp, Pisum sativum
  source 1..201: mol_type = genomic DNA
  variation 101: note = g or a
tataatcatgtactccawgaacactccagtcrtaatakacacaccaaatgacatcactaaatactcatagtgaccatatctcattctgaatgtagtcttcnraatatcgtctgacttcacatgaatctgatgataaccaaaacacaaatcaatcttgttaaacacacaagcmccaaccaattgatccatcagatyatygat

SEQ ID NO: 102 - DNA, 201 bp, Pisum sativum
  source 1..201: mol_type = genomic DNA
  variation 101: note = c or t
catagagatgatatgttggctcataagggtattcaatcaataaagacaaccaatgcaaatagggatsattgggtgactgttaaaatagttgggactctttntgtcataaacaccatcgaagtcrtaactcaggccaacatcataataggaaatacatcrcattgagtgattcttctagtaggtccagatgaragggtgtgt

SEQ ID NO: 103 - DNA, 201 bp, Pisum sativum
  source 1..201: mol_type = genomic DNA
  variation 101: note = a or c
tyrgcttacatakccgaaaacaagttaaaartggccttagaggccraagtaaartacatggttgtcgaagccaaagagactaaagtacaccttgaagggtntggtgtggattwtgaaccaaagccccctgataaaaamyatgtcgagctgactagctgttccttagcttcggtgaatccacactctctctctctctctagy

SEQ ID NO: 104 - DNA, 201 bp, Pisum sativum
  source 1..201: mol_type = genomic DNA
  variation 101: note = t or c
gctaagtcakgtattccagtcgatgcwaggaatcrtcactraaattagttrtgttttcccccacaaagtcrggtattctagttgtcgctaagaatcatcantgaattcacttggtcaatcctcartagatatcaagtattccagctattgctwggaatcgtcactgaatctatattttcccaycagatttaggyrttccag

SEQ ID NO: 105 - DNA, 201 bp, Pisum sativum
  source 1..201: mol_type = genomic DNA
  variation 101: note = a or g
tccaaaacaggtggtttgttgattgatcctccatattyaattgtctttatttgagcataatttatcttccctagagctcacccaataggctagggtgtctnctctgatgccaactgaaattatgttcctcaggggatagatgtcgaacatgatactccgacaaaaggttckacaatatacatcakaaccactataataaca

SEQ ID NO: 106 - DNA, 201 bp, Pisum sativum
  source 1..201: mol_type = genomic DNA
  variation 101: note = g or a
aaaakgtcaraccaagtttawaataaagtctttgacaagtaataagacatgtccargctttatatttttaattaggcatggcctatttaagtaaagctcgnctcrgtccgacctatttcasccctacttyttcccttcacaaattacaacaaaccttaagtttcactattcactttctttattcttrccgyctccatcgtt

SEQ ID NO: 107 - DNA, 201 bp, Pisum sativum
  source 1..201: mol_type = genomic DNA
  variation 101: note = a or g
tgctctggaaactttatcttgyatgattgttgtgaaccaatggaatgatgtacccataataaatgttatgcgccttgacagacctgctcatgtgtttgctncaaaaggggtcactgatgacaaaccatggtatcatgatattaaatgctttatccaaagacaagagtacccacctggggcatcaaacaaagataagaagac

SEQ ID NO: 108 - DNA, 201 bp, Pisum sativum
  source 1..201: mol_type = genomic DNA
  variation 101: note = t or c
taaccytactctgatagtggtaagtctaaccacsagagakaatatttcattgaaatyaattcctttcttttgagcatatcctttaacaaccaytcttgcangatatctttccacttgatcattactatcacrctaatcttatagacacatttgttgyygatggctttccgaccytctggaagtacaayaagatccyatgtc

SEQ ID NO: 109 - DNA, 201 bp, Pisum sativum
  source 1..201: mol_type = genomic DNA
  variation 101: note = a or g
gtatttttaaaggagctcgcattagcaaactacctcattcaccatctccaccaaaaagttcattcattgacgagatatcggtttctatgcacatatatatntcgaacagatcatcaattttgagggcraygataattgcgrttttcgarcgatttcgrctttgytcgrtaaaggagacaccatcaacttatccatgagttg

SEQ ID NO: 110 - DNA, 201 bp, Pisum sativum
  source 1..201: mol_type = genomic DNA
  variation 101: note = a or c
aaagaaaaaggggtgaraattcacatcaataacgacataarmtggaaggtagarttcaaacatctatacattcartaayaatcacacaacatcattatcanacccaatccatcaacatacataaaacaatcatatattcatcatttaaawttatgcaatgrgmctcacaacctcaactagactcatatgcacgtggtacct

SEQ ID NO: 111 - DNA, 201 bp, Pisum sativum
  source 1..201: mol_type = genomic DNA
  variation 101: note = c or a
atagtgcaaaacatgcctttgagaattttgccaaattggtccaacttcaagcccttctgttttratgatgcaagcccyaaatgggaaaacctccaacrtcnaagttgtatatcttttcaataccatcaaaatggacttaaattttgcatcatttggattttttatgaaggagttatgggcacttgaagttggacttttttg

SEQ ID NO: 112 - DNA, 201 bp, Pisum sativum
  source 1..201: mol_type = genomic DNA
  variation 101: note = a or t
gcatgatgaactaataaaaatatryctaagctattatatgwtcatggggtggggtgggggggggcatttgacccacaatggggggtgtaagcaatacaacntaaatggaaatggaaagaawtatacaagttacaaactatgtataaygyctaaaacatacaagtgrcatgayacttcaaacaatacaagtcgtgaatgagt

SEQ ID NO: 113 - DNA, 201 bp, Pisum sativum
  source 1..201: mol_type = genomic DNA
  variation 101: note = t or c
aaaatmtctctctctctctattccctctcaattttcactctccctatcatttcattcaattcacaaactcgaccctcatatttcagctctttcatattcanatgtttactctctctgctaattcaatccaattcaatcctcaagatcactctctcagcrtgaaactctcagaagaagaagtctcagakataggaagaaaaa

SEQ ID NO: 114 - DNA, 201 bp, Pisum sativum
  source 1..201: mol_type = genomic DNA
  variation 101: note = c or t
tcatgtaggactgmacatgaaagatttgaagtcgtrcaaaggtgggaactttttggaattcaatgactcttcgactcgtccatgtgraaccataraccacnagtctcgyttgaagaaggtaaaaataagataactatgaaagtgtttttcctcatcatcccttgtgaaakcatctacaatgacatcttaggaaggtcattt

SEQ ID NO: 115 - DNA, 201 bp, Pisum sativum
  source 1..201: mol_type = genomic DNA
  variation 101: note = g or a
ttgtggctggaattgacaatccagyrgtgaaaaggaagaattttcttttctccaaaattgcttyggtaraactytgtggaagttgctacatatggagytgnagtttctattgccttcaaacatatcaaggctcaaataaggaactagtgggacttaggataagttgtcgtgaaacggacgcttggagatagttaattgatt

SEQ ID NO: 116 - DNA, 201 bp, Pisum sativum
  source 1..201: mol_type = genomic DNA
  variation 101: note = c or t
ctygcaataaggtagctacaatcaaaggtttgttcggagcgtttgaagcayttggttcaactcgaaccaaggggagagaagtaaatakgatctacaamaanacttaggtttgmttggtattgatcatttcttctcttgtataaactttgcaawtgataataaatatctcaattctagttttaraattrrgggcagacgtac

SEQ ID NO: 117 - DNA, 201 bp, Pisum sativum
  source 1..201: mol_type = genomic DNA
  variation 101: note = g or a
aatgtaatttagttgaagtagaaatgactggtaaataattcacttggagtargccttgtactagtaatctaattgtctatcgcaagttggatagagcactngttaacgttgcwtggygcatggctttccctggtgcatattttcrggtgttgtgtaawtttcattctaatcacaacctcattctccttagatgtggccttm

SEQ ID NO: 118 - DNA, 201 bp, Pisum sativum
  source 1..201: mol_type = genomic DNA
  variation 101: note = t or c
atagttaattaatagctaattaacaatttttttcatatcacatcctaattaaaactagtcttgagatggttgtgtaggttggggtgcctgtgatgarcttncaatctctattgcacatgtctctktagtgtttgtagtctttgtcttttttctttttttttcttcataatttgtagtgtttgtagtattttccttcttatt

SEQ ID NO: 119 - DNA, 201 bp, Pisum sativum
  source 1..201: mol_type = genomic DNA
  variation 101: note = t or g
agccaagggtctgatgcttgtctcygcactaaaawtaacsccaacaaatcaaatcatcttttayctcagtgyactcttcaaaymtttmaaaaagracgtgntacttccrctctaccgtgaacgrcgcttaagcctccatgtgtgagcaastaatgtttaactgctagagtgtgatccaagcagattgataaacaaccaaac

SEQ ID NO: 120 - DNA, 201 bp, Pisum sativum
  source 1..201: mol_type = genomic DNA
  variation 101: note = t or c
aaaatggtacaaataaaaatttattaagaaaaaaaataacaaccaacaaatcaacaaaacatacaaagtagatcacaaaaaacacccaagcatataaaaancaatctcgtagaatttaggttagagctcatactrccataaccaccataaatagatcataattcacaaagagagtaacgaaaycacaaaccaaatcctatc

SEQ ID NO: 121 - DNA, 201 bp, Pisum sativum
  source 1..201: mol_type = genomic DNA
  variation 101: note = c or t
mccttttggrtgttcaaatacacatmttyatttatagccatttaggaaggcacttttaacaaccatttggaatagctygawctttaagagacatgycattncawgtaacaatctgatagactccaggmgagctactrgagmaaaggtttcatcaaagtctactccttccayttgggtgtatccttgagcaacaagtctgrc

SEQ ID NO: 122 - DNA, 201 bp, Pisum sativum
  source 1..201: mol_type = genomic DNA
  variation 101: note = c or g
atcaaaaaacataaatgaagatgatgtcgattcaacgcagtatagaagactcattggatcactttgatatgtcacacaaggcctaatatarcatagtgtangtatggtgagaagattcatgcagaagctaaatgtatcacacctagcaactacggagaggatactaaggcatctgaaaggaactcttgacaatgaaattta

SEQ ID NO: 123 - DNA, 201 bp, Pisum sativum
  source 1..201: mol_type = genomic DNA
  variation 101: note = g or c
aatcttgcacttttcgaccagtgaaaactgacccctggtcggttgtaatagtttatgggatgccaaacctgcagatgatgtaactcaggacaaagtctatnacagcctcttggtccacatttgttaatgctacggcttcgacccattttgtgaaataatcgataccgactaacacatacctttgttgtttcgacgaggctg

SEQ ID NO: 124 - DNA, 201 bp, Pisum sativum
  source 1..201: mol_type = genomic DNA
  variation 101: note = g or a
gccarcayttctgaaaccaagttmttatggtttgagctttcatatgatgcaagtatttggatccacttgagttcatytgctacatggtctggatgcaactntaactttgaacctgggtctaatgccttattcagggycaatcaagaartatgtcatctgatacttgggaagatcaagaagaagattgtgaagttgcttgct

SEQ ID NO: 125 - DNA, 201 bp, Pisum sativum
  source 1..201: mol_type = genomic DNA
  variation 101: note = a or g
atggccttaggtttatatgttatggtgtagcgcttcaacctagggtgctcgagtgtactcaaatatcgttatgtttttcgaggtatcacttggactgttgnttttttaccttccatttgctktcattctacaacattgatttgtttcatctagtccattatataagggttgctcggacttttcgaacaattttgtgttccc

SEQ ID NO: 126 - DNA, 201 bp, Pisum sativum
  source 1..201: mol_type = genomic DNA
  variation 101: note = g or t
cctaaacctcttgtcaaacaaatgcatcaaaagggtatcaaaactcaaagtcagsgggcaaggttccacacataaagacatgacatacatactaaaacatntgcaaaaggattggtttcaacattcaaaggaagtgacacaagtgcctcatgctaagcaagcatcaaaataaggactaaaatgcaaacatcaacttcaaaa

SEQ ID NO: 127 - DNA, 201 bp, Pisum sativum
  source 1..201: mol_type = genomic DNA
  variation 101: note = t or c
atacaacaacaastaacattaataagttactttmatgatttyytaacaaacattaaacatttttaacaaggaattactagagaaagattacactaagaaancaaccttgaaayattttcatgcycttkctttcattaccattgtattcattatgttaaagtttatatttcaccactttgcatccaatrtggataattrtgg

SEQ ID NO: 128 - DNA, 201 bp, Pisum sativum
  source 1..201: mol_type = genomic DNA
  variation 101: note = g or a
agcatcaatctgcttctgcacttcctctttgatcttcactgccatatcaggatgagttcttctcaatttctgcttgactggcgggcattctcacttcaacngcaatytatgctccacaatctcaaaatccaaaccagacatgtcttgataggaccaagcaaacacatcagaatactctcgaagaagatcaatcaacccatt

SEQ ID NO: 129 - DNA, 201 bp, Pisum sativum
  source 1..201: mol_type = genomic DNA
  variation 101: note = g or a
agttaattggattaaaagagtttattattgaaaatttcyggktttctttttggcattttgattctcaataytaatcrgggmaaacctccacttcgtcggtnatcsttcacctcttccacactcttgccatgaattatggatgtttgcgtgtgattaaattaagccactgttgtgatttgcttattagaatattcacttatt

SEQ ID NO: 130 - DNA, 201 bp, Pisum sativum
  source 1..201: mol_type = genomic DNA
  variation 101: note = a or g
ataacactcgtgtaccaatgyacataccaaatagaaraaaagtttaaagaaaaaaagtargatttgrtacataccatacaagtttcacacacataccggtngcargtacatacccgatactgatactttgtctaaaatgacctatcaatgyttcacatrttctcacccattatgcatgtttcaaacttccacgataaattt

SEQ ID NO: 131 - DNA, 201 bp, Pisum sativum
  source 1..201: mol_type = genomic DNA
  variation 101: note = g or a
agttggagtctaaggaaaaagatgcacttattaagctacttgaagaccgagtgasaaagagatagagagagccaraggtktcatcctctagcatgcctyanccttccgctgcttggaagaagattgytgatcagcttgtcctcgagaagactcagatgaaggcttcttttgagaccgagatycgtcgcattcgaaggaagt

SEQ ID NO: 132 - DNA, 201 bp, Pisum sativum
  source 1..201: mol_type = genomic DNA
  variation 101: note = t or g
artttctgaataatmatttgrttagytgtamtttaagctggattattygtaatgatatcctctttaatggygggatgagagggttccmraaattgtyttgntggctaaattgrttttttgggattggtttrgagttagatttaaggacattcttccatatsttaggttttrtatttgagttaaatatcctyttamtacrat

SEQ ID NO: 133 - DNA, 201 bp, Pisum sativum
  source 1..201: mol_type = genomic DNA
  variation 101: note = g or a
agcactcttaccgaacagatcctttcccctcaaggttttcaactcctttcgcagctcaaggaattggtctttcatttcatccatcttctcataaacatccngaccttcagatggctcgaaatgatagatggtgtcctcaacgcggggcaaagtatgaataactgttggaggcacagacatgatygggctagatgtcggcat

SEQ ID NO: 134 - DNA, 201 bp, Pisum sativum
  source 1..201: mol_type = genomic DNA
  variation 101: note = t or c
wtgaacctcgttggaagcccaaactggctctkttgttgttgtcggasacctctatcatttggccccactgatcagaattaccatcttcaacaatcttytgngcatctttaaatraggacatggstkccccaattctcttttccttagcaatagacaaggcttggaacggagttccaatctcatcttyaggttctacatayg

SEQ ID NO: 135 - DNA, 201 bp, Pisum sativum
  source 1..201: mol_type = genomic DNA
  variation 101: note = g or a
ggttytgagatgraacaccttccctatttatagggaaggtgttgggatggatargkgatagtaagggcgaggtttgattagagattgcaagtgacttgaancatgctcaaaagtkggatwtttgggataaggattggcgtkttctgacytggtttgcatcaccaaccaactggtttttcwccttagcatcaaacaagtgca

SEQ ID NO: 136 - DNA, 201 bp, Pisum sativum
  source 1..201: mol_type = genomic DNA
  variation 101: note = g or a
gtcacttcgatagctttgtcrcacaaggtctaacttagcatacagtgtaggtakgatgagtagattcatgcaaaagccaaaggtatcacatctagcagcgntgaagaggatactaaggtatctaaaaggaactcacractatggcattttgtttcctgcagctgatgaaggaaaataatrcaaattagtgggatacaccga

SEQ ID NO: 137 - DNA, 201 bp, Pisum sativum
  source 1..201: mol_type = genomic DNA
  variation 101: note = c or t
caatgaacaccmaaacatctcatcaccayaacactcaatcattgtttgactttagtacaccacragaaccattactttgtttccaaaacgattcaatgtcncaattcgggcaaccataccgtccataattcacccaaccacaacgacccaactacgacaacatggacatcgaactcaattacggragcaccgttggcgaca

SEQ ID NO: 138 - DNA, 201 bp, Pisum sativum
  source 1..201: mol_type = genomic DNA
  variation 101: note = c or t
aattaaaaagaaaaatagaaaaaatgagggccatcagatcyccctyattaaytgaggtggcagatctgatggccaagcgcgysctatctaccatggtcctnaatcaatgcgtcacakgagtgrtaatcagaaggagcggmtcagattaaaayatttcaaacaaatcaaatggccaagagacgcgccaacgcatcamcgrag

SEQ ID NO: 139 - DNA, 201 bp, Pisum sativum
  source 1..201: mol_type = genomic DNA
  variation 101: note = t or g
tgcayatatatatacmttgttcgagcttctccaagtcttccaagtccttttcawtatccactttcaaagggggagatcttaamcaattttgtgcacatatnaaagcttcaacaatgtatggtgtaagagaaatcctataacaactaagaactctaccccctgtgctaaaagcagactctraagyaacagtagatattggca

SEQ ID NO: 140 - DNA, 201 bp, Pisum sativum
  source 1..201: mol_type = genomic DNA
  variation 101: note = g or a
atctgaatttactagggktactcctaatggtgatgttaatcatgatcaaaktacatcttaaaagaataattgtattgctaaacctccaacattcagtgganaatctactgaatttgaatggtggaakagcaagatgtacactaaaatcataggtcttgatgatgggttatgrgatatgttagaagatggmatttacatcaa

SEQ ID NO: 141 - DNA, 201 bp, Pisum sativum
  source 1..201: mol_type = genomic DNA
  variation 101: note = c or g
cttratcttgagwtkctttrrttatcatcaatgttgaaggckattttcatgygttrtcgtgatcaaaactccagatgtcgatcttcattacttscaagcanatagattcacatactgcccyggcaccccyaagtcrcctacctacaacaaacagktgggcctataacatgtttraaattttmacttattgtgtktctctta

SEQ ID NO: 142 - DNA, 201 bp, Pisum sativum
  source 1..201: mol_type = genomic DNA
  variation 101: note = a or g
cctaaatctcccacctyccgccayaygagcctctsgttaaccattgtataaatcatcaccatratgtcscttctctcatgcatatccttcatccagcatancatgaagccttgaaaataaacaagcaaagctaaagtccaattcttattgkcacattgraggccctacacaaaaagagtatcaccyttgtaaataccataa

SEQ ID NO: 143 - DNA, 201 bp, Pisum sativum
  source 1..201: mol_type = genomic DNA
  variation 101: note = 143
ctcagatggttggtgctgacaaccatgagaycaacyagggacawrctcttyakacttctrtgycagttactattgaaactcctgatgataacaaagatkantacaaaggtcctcaccttcattttcatgttcctcctcagtctgctaaagctactattcaaaatttgaatcaaggtgttcagatacctcctccttattctr

SEQ ID NO: 144 - DNA, 201 bp, Pisum sativum
  source 1..201: mol_type = genomic DNA
  variation 101: note = t or c
ttaygaggatctttggtggagttaagggagttagggccaccccaagcggtccttcctcccgtttctgatgtcaacgsttggtgcgagttccattytggcgntcccggtcattcaatcgagaactgtaaatccttaatgtataaaatgcaagattcgatagattctaaggcattgcgtttgcgcctaacatcccaaatgtaa

SEQ ID NO: 145 - DNA, 201 bp, Pisum sativum
  source 1..201: mol_type = genomic DNA
  variation 101: note = a or g
gaatttgttgaccttytgtgatgaacacatcccagaagaattctagcccaaacctttagcttagggtgaaaatccttgaccttrctagagtgttttcgagngacgaaartcacttcggaaatctttgtcaaattggattccttttccactagttgttcacatmtgattctagacccatcatatccaatgagtttggcaata

SEQ ID NO: 146 - DNA, 201 bp, Pisum sativum
  source 1..201: mol_type = genomic DNA
  variation 101: note = a or g
ttattgttgacccatatgayatctcttgtattytatrtgggtatttyctagaatcagtctctcatttgtttrttacctrtgttttagcttcgaagatttgntatatgattttcaagtggttgtggtgggtggtggttatccmtagggatcmtaggattctttttkagattttacatctttakgtgatrgggmtaagctgag

SEQ ID NO: 147 - DNA, 201 bp, Pisum sativum
  source 1..201: mol_type = genomic DNA
  variation 101: note = g or a
atcgagaaaagtcccaataaatataaaaatgytctctcttgytgaacycctaaataaatgtcccaacaatggtatcaaagcccrgttcgattgaargacynrctcacttgtatyraaagtactccccataggtggtgggtaggaggtgtcccgttgaagagtgtcaacggtggtacaagtgtatrtggaagaaagaactta

SEQ ID NO: 148 - DNA, 201 bp, Pisum sativum
  source 1..201: mol_type = genomic DNA
  variation 101: note = g or a
cgtragtaggattaggcaatrcaacataacrttaattcactagccaytactcggggtctcctatcyctattcaaccaacataagcacaacaattrcccwangtctaacacatagactaatggtcagtattycctatgtgcccaaaattagatkaaaagagtatctcacataaaaarcattaygaacaagaagaaattgaat

(19) United States
(12) Patent Application Publication
(10) Pub. No.: US 2025/0359526 A1
(43) Pub. Date: Nov. 27, 2025

(54) Compositions and Methods for Producing High-Protein Pea Plants

(71) Applicant: Confluence Genetics, LLC, St. Louis, MO (US)

(72) Inventors: Janice Kofsky, St. Louis, MO (US); David Larson, St. Louis, MO (US); Herbert Wolfgang Goettel, St. Louis, MO (US)

(21) Appl. No.: 19/219,689

(22) Filed: May 27, 2025

Related U.S. Application Data
(60) Provisional application No. 63/651,818, filed on May 24, 2024.

(51) Int. Cl. A01H 1/04; A01H 1/00; C12Q 1/6827; C12Q 1/6895

(52) U.S. Cl. CPC A01H 1/045; A01H 1/108; C12Q 1/6827; C12Q 1/6895; C12Q 2600/13; C12Q 2600/156

(57) ABSTRACT

Provided herein are methods for producing pea plants having high protein using marker-assisted selection. The disclosure further provides methods for introgressing one or more loci comprising at least a high-protein allele linked to the high-protein QTL, thus producing high-protein pea plants.

### RELATED APPLICATIONS

This application claims priority to U.S. Provisional Application No. 63/651,818, filed on May 24, 2024, the content of which is incorporated herein by reference in its entirety.

### FIELD OF THE INVENTION

This disclosure relates generally to the field of agricultural biotechnology. More specifically, this disclosure relates to methods for producing pea plants or seeds with high protein content. Also provided herein are compositions for use in such methods.

### SEQUENCE LISTING

This application contains a Sequence Listing which is submitted herewith in electronically readable format. The Sequence Listing file was created on May 27, 2025, is named “B88552_1610_SL.xml” and its size is 212,056 bytes. The entire contents of the Sequence Listing in the XML file are incorporated by reference herein.

### BACKGROUND OF THE INVENTION

Pea is an excellent source of protein and supplies adequate and nutritious food and feed for use. Most commercially produced peas are processed to produce edible oil and one or more protein products. Pea protein is valued for its high nutritional quality for people and livestock, and for functional properties, such as gel and foam formation. The initial protein fraction is a pea meal which is often further processed to produce more highly refined protein products, primarily pea protein concentrates or pea protein isolates. Alternative processing methods produce protein-based pea foods. There is a great need to obtain pea varieties that possess high protein content. However, negative correlations between high protein content and high yield are often observed. Thus, peas with higher content of protein are desirable and would represent a substantial commercial value that would benefit farmers, breeders, food processors, and consumers alike.

### SUMMARY OF THE INVENTION

The present disclosure identifies genetic loci conferring high protein phenotype in pea, and provides molecular markers linked to these high protein loci. This disclosure provides methods of producing a population of high-protein pea plants or seeds. Further provided are methods of introgressing a high-protein QTL, thereby a progeny plant or seed comprising a high-protein allele of a polymorphic locus linked to the high-protein QTL. The genetic loci, markers, and methods provided herein therefore allow for production of new varieties of pea plants with high protein content.

Accordingly, in a first aspect, provided herein is a method of producing a population of high-protein pea plants or seeds. The method includes (i) genotyping a first population of pea plants or seeds for the presence of at least one high-protein molecular marker that is within 20 centimorgans of one or more high-protein Quantitative Trait Locus (QTLs) selected from the group consisting of Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps02_432513197, Ps02_440456554, Ps03_158264810, Ps03_205819517, Ps03_206829164, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps06_410567663, Ps06_427519500, Ps06_446483044, Ps07_9801763, Ps07_20773355, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897; (ii) selecting from the first population one or more pea plants or seeds comprising one or more high-protein alleles having the at least one high-protein molecular marker; and (iii) producing a second population of progeny pea plants or seeds from the selected one or more pea plants or plants grown from the selected seeds. The second population of progeny pea plants or seeds comprises the one or more high-protein alleles having the at least one high-protein molecular marker, and the second population of progeny pea plants or seeds are high-protein pea plants or seeds, thereby producing a population of high-protein pea plants or seeds.

In some embodiments, said at least one high protein molecular marker is within 10 centimorgans of said one or more high protein QTLs.

In some embodiments, the one or more high-protein molecular markers confer a yield penalty of less than 5% under normal growing conditions.

In some embodiments, genotyping comprises assaying a single nucleotide polymorphism (SNP) marker.

In some embodiments, genotyping comprises the use of an oligonucleotide probe or a pair of primers. In some embodiments, the oligonucleotide probe or the pair of primers comprise a nucleic acid molecule that comprises at least 15 nucleotides that include or are immediately adjacent to the SNP or the deletion sequence, wherein the nucleic acid molecule is at least 90 percent identical to a sequence of the same number of consecutive nucleotides in either strand of DNA that include or are immediately adjacent to the SNP or the deletion sequence.

In some embodiments, genotyping comprises detecting a haplotype.

In some embodiments, said one or more high protein QTLs are Ps03_531239107 and/or Ps05_49389403. In some embodiments, said one or more high protein QTLs are Ps03_531014546, Ps03_531232613, and/or Ps03_531239107.

In some embodiments, pea plants or seeds comprising said one or more high-protein alleles have protein content that is greater by at least 1.0% dry weight relative to pea plants or seeds without said one or more high-protein alleles.

In some embodiments, the resulting population of high-protein pea plants or pea seeds comprises at least 20%, 21%, 22%, 23%, 24%, 25%, 26%, or 27% protein by weight.

In some embodiments, the pea plants or seeds comprising one or more high-protein alleles have yield that is 99% or greater relative to pea plants or seeds without said one or more high-protein alleles.

In some embodiments, the second population of progeny pea plants or seeds further comprise one or more alleles associated with high yield.

In some embodiments, the method further comprises determining the protein content of the second population of pea plants or seeds, wherein the second population of pea plants or seeds having the one or more high-protein alleles have an increased level of protein when compared to a control population of pea plants or seeds lacking the one or more high-protein alleles.

In one aspect, provided herein is a high-protein population of pea plants produced by the method provided herein.

In some embodiments, said high-protein population of pea plants has a greater frequency of the at least one high-protein molecular marker than said first population of pea plants.

In one aspect, provided herein is a method of introgressing a high-protein QTL. The method comprising: (a) crossing a first pea plant comprising a high-protein QTL with a second pea plant of a different genotype to produce one or more progeny plants or seeds; and (b) selecting a progeny plant or seed comprising one or more high-protein alleles of a polymorphic locus linked to the high-protein QTL. The polymorphic locus is a chromosomal segment comprising any marker within genomic regions 421,829,254-437,541,609 of chromosome 3, 1-54716217 of chromosome 5, 20877277-371072249 of chromosome 1LG6, 10842575-426699364 of chromosome 2LG1, 104891818-425968089 of chromosome 3LG5, 5972665-445125850 of chromosome 4LG4, 362278-547326524 of chromosome 5LG3, 1621846-438943399 of chromosome 6LG2, 8316015-481276628 of chromosome 7LG7, scaffold 02116, scaffold 04655, scaffold 00066, scaffold 03789, scaffold 02021, scaffold 00644, scaffold 00254, scaffold 02127, scaffold 00706, super-scaffold 888, scaffold 00462, scaffold 02449, scaffold 02833, scaffold 00839, scaffold 05469, scaffold 06512, scaffold 02959 scaffold 00840, or scaffold 01757 of a Pisum sativum genome.

In some embodiments, the Pisum sativum genome is the Cameor v1a reference genome. In some embodiments, said high-protein QTL comprises a SNP marker associated with high protein content.

In some embodiments, the SNP marker is selected from the group consisting of Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps02_432513197, Ps02_440456554, Ps03_158264810, Ps03_205819517, Ps03_206829164, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps06_410567663, Ps06_427519500, Ps06_446483044, Ps07_9801763, Ps07_20773355, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897.

In some embodiments, the SNP marker is selected from the group consisting of: a C at position 425968088 of chromosome 3; a C at position 563531992 of chromosome 5; a C at position 101 of SEQ ID NO: 47 in a genomic region comprising the nucleic acid sequence of SEQ ID NO: 47, or at a corresponding position of a genomic region at least 50 nucleotides of which is aligned to SEQ ID NO: 47 for at least 90% sequence identity; an A at position 36835261 of chromosome 5; an A at position 101 of SEQ ID NO: 148 in a genomic region comprising the nucleic acid sequence of SEQ ID NO: 148, or at a corresponding position of a genomic region at least 50 nucleotides of which is aligned to SEQ ID NO: 148 for at least 90% sequence identity; a G at position 36095 of scaffold 04655; a T at position 108299170 of chromosome 1LG6; a G at position 157869 of scaffold 00066; a T at position 23108565 of chromosome 1LG6; an A at position 122338117 of chromosome 1LG6; a G at position 306857129 of chromosome 1LG6; a G at position 45686305 of chromosome 1LG6; an A at position 371072249 of chromosome 1LG6; a G at position 72083191 of chromosome 1LG6; a C at position 79225752 of chromosome 1LG6; a T at position 8290 of scaffold 02021; an A at position 119392808 of chromosome 2LG1; a T at position 10842575 of chromosome 2LG1; an A at position 169314375 of chromosome 2LG1; a G at position 286755665 of chromosome 2LG1; a G at position 91532 of scaffold 00644; a C at position 301660243 of chromosome 2LG1; an A at position 420361771 of chromosome 2LG1; a G at position 426699364 of chromosome 2LG1; a C at position 26206979 of chromosome 2LG1; a G at position 30219372 of chromosome 3LG5; an A at position 393751811 of chromosome 3LG5; an A at position 417958980 of chromosome 3LG5; a G at position 421049387 of chromosome 3LG5; a T at position 83578489 of chromosome 4LG4; a C at position 109277412 of chromosome 4LG4; an A at position 117043426 of chromosome 4LG4; a G at position 163719335 of chromosome 4LG4; a T at position 18486554 of chromosome 4LG4; a C at position 247901046 of chromosome 4LG4; a T at position 2191851 of chromosome 4LG4; a C at position 444278355 of chromosome 4LG4; a T at position 445125850 of chromosome 4LG4; a T at position 5972665 of chromosome 4LG4; an A at position 96025751 of chromosome 5LG3; an A at position 12104 of scaffold 00462; a C at position 178039871 of chromosome 5LG3; an A at position 132883215 of chromosome 5LG3; a G at position 24130766 of chromosome 5LG3; a T at position 228797264 of chromosome 5LG3; a G at position 239060496 of chromosome 5LG3; a C at position 2288318 of chromosome 5LG3; a G at position 331834371 of chromosome 5LG3; a T at position 50774 of scaffold 02833; a C at position 37349400 of chromosome 5LG3; a G at position 39703 of super-scaffold 888; a G at position 509926370 of chromosome 5LG3; a C at position 509729669 of chromosome 5; an A at position 522716439 of chromosome 5LG3; a T at position 124873928 of chromosome 5LG3; a G at position 551226342 of chromosome 5LG3; an A at position 547326524 of chromosome 5; a T at position 1621846 of chromosome 6LG2; an A at position 4002 of scaffold 00839; a T at position 374758162 of chromosome 6LG2; a T at position 401325650 of chromosome 6LG2; a C at position 426328393 of chromosome 6LG2; a G at position 438943398 of chromosome 6; a T at position 72341 of scaffold 02959; a G at position 89032441 of chromosome 7LG7; an A at position 19382049 of chromosome 7LG7; a C at position 310437720 of chromosome 7LG7; a T at position 310515874 of chromosome 7LG7; a C at position 335690162 of chromosome 7LG7; an A at position 322450055 of chromosome 7; a G at position 10989 of scaffold 00840; an A at position 460750292 of chromosome 7LG7; a G at position 13304 of scaffold 06512; a T at position 52311972 of chromosome 7; a G at position 50802012 of chromosome 7LG7; an A at position 56383957 of chromosome 7LG7; a G at position 1311773 of chromosome 7LG7; a G at position 8316015 of chromosome 7LG7; a C at position 36097 of scaffold 04655; an A at position 20877277 of chromosome 1LG6; a T at position 194893633 of chromosome 1LG6; a G at position 72152 of scaffold 03789; an A at position 30542636 of chromosome 1LG6; a T at position 116776833 of chromosome 1LG6; an A at position 288453266 of chromosome 1LG6; a G at position 367968951 of chromosome 1LG6; a T at position 51330566 of chromosome 1LG6; a C at position 29379 of scaffold 02116; a G at position 87185358 of chromosome 1LG6; a G at position 5787797 of chromosome 2LG1; a C at position 87090294 of chromosome 2LG1; an A at position 383268619 of chromosome 2LG1; a C at position 104891818 of chromosome 3LG5; a G at position 72342 of scaffold 00254; a T at position 173063548 of chromosome 3LG5; a T at position 174636272 of chromosome 3LG5; a C at position 396332351 of chromosome 3LG5; a G at position 423551062 of chromosome 3LG5; a C at position 827484 of chromosome 3LG5; an A at position 425962517 of chromosome 3; a T at position 94928513 of chromosome 4LG4; an A at position 47049 of scaffold 02127; a C at position 165487268 of chromosome 4LG4; an A at position 165597701 of chromosome 4LG4; an A at position 218884465 of chromosome 4LG4; a G at position 228522691 of chromosome 4LG4; a C at position 352524054 of chromosome 4LG4; an A at position 363782042 of chromosome 4LG4; a G at position 285377934 of chromosome 4LG4; a G at position 389689436 of chromosome 4LG4; an A at position 145804 of scaffold 00706; an A at position 374997960 of chromosome 4LG4; a G at position 418184353 of chromosome 4LG4; an A at position 420833970 of chromosome 4LG4; an A at position 134409547 of chromosome 5LG3; an A at position 20543180 of chromosome 5LG3; a G at position 217510948 of chromosome 5LG3; a C at position 84199 of scaffold 02449; a G at position 234213508 of chromosome 5LG3; a T at position 236504935 of chromosome 5LG3; a C at position 268153046 of chromosome 5LG3; a T at position 278088295 of chromosome 5LG3; an A at position 84459019 of chromosome 5LG3; a G at position 306598116 of chromosome 5LG3; a G at position 413429268 of chromosome 5; a G at position 35018191 of chromosome 5LG3; a C at position 362278 of chromosome 5LG3; a C at position 492763269 of chromosome 5LG3; a G at position 499899891 of chromosome 5LG3; a T at position 39908055 of chromosome 5LG3; a T at position 143390359 of chromosome 5LG3; a G at position 535824046 of chromosome 5LG3; an A at position 89382481 of chromosome 6LG2; a T at position 111705293 of chromosome 6LG2; an A at position 191916418 of chromosome 6LG2; a G at position 303558968 of chromosome 6LG2; a T at position 406586297 of chromosome 6LG2; a C at position 17855 of scaffold 05469; an A at position 62010766 of chromosome 6LG2; a T at position 64688438 of chromosome 6LG2; a C at position 75309486 of chromosome 6LG2; a T at position 86301013 of chromosome 6LG2; an A at position 45871271 of chromosome 7LG7; an A at position 16557044 of chromosome 7LG7; a T at position 223304507 of chromosome 7LG7; an A at position 158981077 of chromosome 7LG7; a C at position 365136400 of chromosome 7LG7; a G at position 47207 of scaffold 01757; an A at position 481276628 of chromosome 7LG7; an A at position 52153701 of chromosome 7LG7; and a G at position 88161594 of chromosome 7LG7 of the Pisum sativum genome.

In one aspect, provided herein is a high-protein population of pea plants or seeds produced by the method provided herein.

In some embodiments, said high-protein population has a greater frequency of the one or more high-protein alleles than said first population of pea plants.

In some embodiments, pea plants or seeds comprising said one or more high-protein alleles have protein content that is greater by at least 1.0% dry weight relative to pea plants or seeds without said one or more high-protein alleles.

In some embodiments, the high-protein population of pea plants or seeds comprises at least 20%, 21%, 22%, 23%, 24%, 25%, 26%, or 27% protein by weight.

In some embodiments, the high-protein population of pea plants or seeds comprising one or more high-protein alleles have yield that is 95, 96, 97, 98, or 99% or greater relative to pea plants or seeds without one or more high-protein alleles.

In one aspect, provided herein is a nucleic acid molecule for detecting a high-protein molecular marker in a Pisum sativum genome, wherein the nucleic acid molecule comprises at least 15 nucleotides that include or are immediately adjacent to the marker, wherein the nucleic acid molecule is at least 90 percent identical to a sequence of the same number of consecutive nucleotides in either strand of DNA that include or are immediately adjacent to the marker.

In some embodiments, the high-protein molecular marker is a SNP marker, and wherein the SNP marker is selected from the group consisting of a C at position 425968088 of chromosome 3; a C at position 563531992 of chromosome 5; a C at position 101 of SEQ ID NO: 47 in a genomic region comprising the nucleic acid sequence of SEQ ID NO: 47, or at a corresponding position of a genomic region at least 50 nucleotides of which is aligned to SEQ ID NO: 47 for at least 90% sequence identity; an A at position 36835261 of chromosome 5; an A at position 101 of SEQ ID NO: 148 in a genomic region comprising the nucleic acid sequence of SEQ ID NO: 148, or at a corresponding position of a genomic region at least 50 nucleotides of which is aligned to SEQ ID NO: 148 for at least 90% sequence identity; a G at position 36095 of scaffold 04655; a T at position 108299170 of chromosome 1LG6; a G at position 157869 of scaffold 00066; a T at position 23108565 of chromosome 1LG6; an A at position 122338117 of chromosome 1LG6; a G at position 306857129 of chromosome 1LG6; a G at position 45686305 of chromosome 1LG6; an A at position 371072249 of chromosome 1LG6; a G at position 72083191 of chromosome 1LG6; a C at position 79225752 of chromosome 1LG6; a T at position 8290 of scaffold 02021; an A at position 119392808 of chromosome 2LG1; a T at position 10842575 of chromosome 2LG1; an A at position 169314375 of chromosome 2LG1; a G at position 286755665 of chromosome 2LG1; a G at position 91532 of scaffold 00644; a C at position 301660243 of chromosome 2LG1; an A at position 420361771 of chromosome 2LG1; a G at position 426699364 of chromosome 2LG1; a C at position 26206979 of chromosome 2LG1; a G at position 30219372 of chromosome 3LG5; an A at position 393751811 of chromosome 3LG5; an A at position 417958980 of chromosome 3LG5; a G at position 421049387 of chromosome 3LG5; a T at position 83578489 of chromosome 4LG4; a C at position 109277412 of chromosome 4LG4; an A at position 117043426 of chromosome 4LG4; a G at position 163719335 of chromosome 4LG4; a T at position 18486554 of chromosome 4LG4; a C at position 247901046 of chromosome 4LG4; a T at position 2191851 of chromosome 4LG4; a C at position 444278355 of chromosome 4LG4; a T at position 445125850 of chromosome 4LG4; a T at position 5972665 of chromosome 4LG4; an A at position 96025751 of chromosome 5LG3; an A at position 12104 of scaffold 00462; a C at position 178039871 of chromosome 5LG3; an A at position 132883215 of chromosome 5LG3; a G at position 24130766 of chromosome 5LG3; a T at position 228797264 of chromosome 5LG3; a G at position 239060496 of chromosome 5LG3; a C at position 2288318 of chromosome 5LG3; a G at position 331834371 of chromosome 5LG3; a T at position 50774 of scaffold 02833; a C at position 37349400 of chromosome 5LG3; a G at position 39703 of super-scaffold 888; a G at position 509926370 of chromosome 5LG3; a C at position 509729669 of chromosome 5; an A at position 522716439 of chromosome 5LG3; a T at position 124873928 of chromosome 5LG3; a G at position 551226342 of chromosome 5LG3; an A at position 547326524 of chromosome 5; a T at position 1621846 of chromosome 6LG2; an A at position 4002 of scaffold 00839; a T at position 374758162 of chromosome 6LG2; a T at position 401325650 of chromosome 6LG2; a C at position 426328393 of chromosome 6LG2; a G at position 438943398 of chromosome 6; a T at position 72341 of scaffold 02959; a G at position 89032441 of chromosome 7LG7; an A at position 19382049 of chromosome 7LG7; a C at position 310437720 of chromosome 7LG7; a T at position 310515874 of chromosome 7LG7; a C at position 335690162 of chromosome 7LG7; an A at position 322450055 of chromosome 7; a G at position 10989 of scaffold 00840; an A at position 460750292 of chromosome 7LG7; a G at position 13304 of scaffold 06512; a T at position 52311972 of chromosome 7; a G at position 50802012 of chromosome 7LG7; an A at position 56383957 of chromosome 7LG7; a G at position 1311773 of chromosome 7LG7; a G at position 8316015 of chromosome 7LG7; a C at position 36097 of scaffold 04655; an A at position 20877277 of chromosome 1LG6; a T at position 194893633 of chromosome 1LG6; a G at position 72152 of scaffold 03789; an A at position 30542636 of chromosome 1LG6; a T at position 116776833 of chromosome 1LG6; an A at position 288453266 of chromosome 1LG6; a G at position 367968951 of chromosome 1LG6; a T at position 51330566 of chromosome 1LG6; a C at position 29379 of scaffold 02116; a G at position 87185358 of chromosome 1LG6; a G at position 5787797 of chromosome 2LG1; a C at position 87090294 of chromosome 2LG1; an A at position 383268619 of chromosome 2LG1; a C at position 104891818 of chromosome 3LG5; a G at position 72342 of scaffold 00254; a T at position 173063548 of chromosome 3LG5; a T at position 174636272 of chromosome 3LG5; a C at position 396332351 of chromosome 3LG5; a G at position 423551062 of chromosome 3LG5; a C at position 827484 of chromosome 3LG5; an A at position 425962517 of chromosome 3; a T at position 94928513 of chromosome 4LG4; an A at position 47049 of scaffold 02127; a C at position 165487268 of chromosome 4LG4; an A at position 165597701 of chromosome 4LG4; an A at position 218884465 of chromosome 4LG4; a G at position 228522691 of chromosome 4LG4; a C at position 352524054 of chromosome 4LG4; an A at position 363782042 of chromosome 4LG4; a G at position 285377934 of chromosome 4LG4; a G at position 389689436 of chromosome 4LG4; an A at position 145804 of scaffold 00706; an A at position 374997960 of chromosome 4LG4; a G at position 418184353 of chromosome 4LG4; an A at position 420833970 of chromosome 4LG4; an A at position 134409547 of chromosome 5LG3; an A at position 20543180 of chromosome 5LG3; a G at position 217510948 of chromosome 5LG3; a C at position 84199 of scaffold 02449; a G at position 234213508 of chromosome 5LG3; a T at position 236504935 of chromosome 5LG3; a C at position 268153046 of chromosome 5LG3; a T at position 278088295 of chromosome 5LG3; an A at position 84459019 of chromosome 5LG3; a G at position 306598116 of chromosome 5LG3; a G at position 413429268 of chromosome 5; a G at position 35018191 of chromosome 5LG3; a C at position 362278 of chromosome 5LG3; a C at position 492763269 of chromosome 5LG3; a G at position 499899891 of chromosome 5LG3; a T at position 39908055 of chromosome 5LG3; a T at position 143390359 of chromosome 5LG3; a G at position 535824046 of chromosome 5LG3; an A at position 89382481 of chromosome 6LG2; a T at position 111705293 of chromosome 6LG2; an A at position 191916418 of chromosome 6LG2; a G at position 303558968 of chromosome 6LG2; a T at position 406586297 of chromosome 6LG2; a C at position 17855 of scaffold 05469; an A at position 62010766 of chromosome 6LG2; a T at position 64688438 of chromosome 6LG2; a C at position 75309486 of chromosome 6LG2; a T at position 86301013 of chromosome 6LG2; an A at position 45871271 of chromosome 7LG7; an A at position 16557044 of chromosome 7LG7; a T at position 223304507 of chromosome 7LG7; an A at position 158981077 of chromosome 7LG7; a C at position 365136400 of chromosome 7LG7; a G at position 47207 of scaffold 01757; an A at position 481276628 of chromosome 7LG7; an A at position 52153701 of chromosome 7LG7; and a G at position 88161594 of chromosome 7LG7 of the Pisum sativum genome.

In some embodiments, the Pisum sativum genome is the Cameor v1a reference genome.

In some embodiments, the nucleic acid molecule provided herein further comprises a detectable label. In some embodiments, said detectable label is a fluorescent label or a radioactive label.

### DETAILED DESCRIPTION OF THE INVENTION

### 1.1. References and Definitions

The present disclosure now will be described more fully hereinafter. The disclosure may be embodied in many different forms and should not be construed as limited to the aspects set forth herein; rather, these aspects are provided so that this disclosure will satisfy applicable legal requirements.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises,” “comprising,” “includes,” “including,” “having” and their conjugates mean “including but not limited to.”

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, “quantitative trait locus” (QTL) or “quantitative trait loci” (QTLs) refer to a genetic domain that effects a phenotype that can be described in quantitative terms and can be assigned a “phenotypic value” which corresponds to a quantitative value for the phenotypic trait.

As used herein, “allele” refers to an alternative nucleic acid sequence at a particular locus. The length of an allele can be as small as one nucleotide base. For example, a first allele can occur on one chromosome, while a second allele occurs on a second homologous chromosome, e.g., as occurs for different chromosomes of a heterozygous individual, or between different homozygous or heterozygous individuals in a population.

As used herein, “locus” is a chromosome region or chromosomal region where a polymorphic nucleic acid, trait determinant, gene, or marker is located. A locus may represent a single nucleotide, a few nucleotides or a large number of nucleotides in a genomic region. The loci of this disclosure comprise one or more polymorphisms in a population; e.g., alternative alleles are present in some individuals. A “gene locus” is a specific chromosome location in the genome of a species where a specific gene can be found.

An allele of a QTL can, as used herein, can comprise multiple genes or other genetic factors even within a contiguous genomic region or linkage group, such as a haplotype. As used herein, an allele of a QTL can therefore encompasses more than one gene or other genetic factor where each individual gene or genetic component is also capable of exhibiting allelic variation and where each gene or genetic factor is also capable of eliciting a phenotypic effect on the quantitative trait in question. In an embodiment of the present invention the allele of a QTL comprises one or more genes or other genetic factors that are also capable of exhibiting allelic variation. The use of the term “an allele of a QTL” is thus not intended to exclude a QTL that comprises more than one gene or other genetic factor. Specifically, an “allele of a QTL” in the present in the invention can denote a haplotype within a haplotype window wherein a phenotype can be disease resistance. A haplotype window is a contiguous genomic region that can be defined, and tracked, with a set of one or more polymorphic markers wherein said polymorphisms indicate identity by descent. A haplotype within that window can be defined by the unique fingerprint of alleles at each marker. As used herein, an allele is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

As used herein, a “haplotype” is the genotype of an individual at a plurality of genetic loci. Typically, the genetic loci described by a haplotype are physically and genetically linked, e.g., in the same chromosome interval. A haplotype can also refer to a combination of SNP alleles located within a single gene.

As used herein, “polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, “SNP” or “single nucleotide polymorphism” means a sequence variation that occurs when a single nucleotide (A, T, C, or G) in the genome sequence is altered or variable.

As used herein, “marker,” or “molecular marker,” or “marker locus” is a term used to denote a nucleic acid or amino acid sequence that is sufficiently unique to characterize a specific locus on the genome

As used herein, a centimorgan (“cM”) is a unit of measure of recombination frequency and genetic distance between two loci. One cM is equal to a 1% chance that a marker at one genetic locus will be separated from a marker at, a second locus due to crossing over in a single generation.

As used herein, “introgression” refers to the transmission of a desired allele of a genetic locus from one genetic background to another.

As used herein, “primer” refers to an oligonucleotide (synthetic or occurring naturally), which is capable of acting as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers may be provided in double-stranded form, though the single-stranded form is more typically used. A primer can further contain a detectable label, for example a 5′ end label.

As used herein, “probe” refers to an oligonucleotide (synthetic or occurring naturally) that is complementary (though not necessarily fully complementary) to a polynucleotide of interest and forms a duplex structure by hybridization with at least one strand of the polynucleotide of interest. Typically, probes are oligonucleotides from 10 to 50 nucleotides in length, but longer or shorter sequences can be employed. A probe can further contain a detectable label.

As used herein, the terms “phenotype,” or “phenotypic trait,” or “trait” refers to one or more detectable characteristics of a cell or organism which can be influenced by genotype. The phenotype can be observable to the naked eye, or by any other means of evaluation known in the art, e.g., microscopy, biochemical analysis, genomic analysis, an assay for a particular disease tolerance, etc. In some cases, a phenotype is directly controlled by a single gene or genetic locus, e.g., a “single gene trait.” In other cases, a phenotype is the result of several genes. In specific embodiments, the phenotype of pea seeds is a high-protein phenotype.

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, pulp, juice, kernels, ears, cobs, husks, stalks, roots, root tips, anthers, and the like. A plant cell is a biological cell of a plant, taken from a plant or derived through culture of a cell taken from a plant. Progeny, variants, and mutants of the regenerated plants are also included within the scope of the invention, provided that these parts comprise the introduced polynucleotides. Further provided is a processed plant product (e.g., extract) or byproduct that retains one or more polynucleotides disclosed herein. A progeny plant can be from any filial generation, e.g., F1, F2, F3, F4, F5, F6, F7, etc. A plant cell is a biological cell of a plant, taken from a plant or derived through culture from a cell taken from a plant.

As used herein, “cross” or “crossing” or “crossed” means to produce progeny via fertilization (e.g. cells, seeds or plants) and includes crosses between plants (sexual) and self-fertilization (selfing). Typically, a cross occurs after pollen is transferred from one flower to another, but those of ordinary skill in the art will understand that plant breeders can leverage their understanding of crossing, pollination, syngamy, and fecundation to circumvent certain steps of the plant life cycle and yet achieve equivalent outcomes, for example, a plant or cell of a pea cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of crossing as used herein when performed in conjunction with these teachings. In certain embodiments, the gametes are not different cell types (i.e., egg vs. sperm), but rather the same type and techniques are used to effect the combination of their genomes into a regenerable cell. Other embodiments of crossing include circumstances where the gametes originate from the same parent plant, i.e., a “self” or “self-fertilization.” While selfing a plant does not require the transfer of pollen from one plant to another, those of skill in the art will recognize that it nevertheless serves as an example of a cross. Thus, methods and compositions taught herein are not limited to certain techniques or steps that must be performed to create a plant or an offspring plant of the claimed invention, but rather include broadly any method that is substantially the same and/or results in compositions of the claimed invention.

As used herein, a “pea plant” refers to a plant of species Pisum sativum L. and includes all plant varieties that can be bred with pea, including wild species such as Pisum fulvum and Pisum sativum subs elatius.

A “high-protein pea plant” or “high-protein pea seed” as used herein refers to a pea plant or pea seed having greater seed protein content than a reference sample of pea plant or seed. In specific embodiments, a high-protein pea population or a high-protein population of pea plants has an average seed protein content of at least 20%, 21%, 22%, 23%, 24%, 25%, 26%, 27%, 28%, 29%, or 30% by weight. In particular embodiment a high protein population comprises an average seed protein content of at least 26.5%, 27%, 27.5%, 28% by weight (dry weight basis). In specific embodiments, a high-protein pea plant or high-protein pea seed has greater seed protein content than a commodity pea seed or commodity pea plant. Commodity peas may have a protein content of less than 40%, or between about 35% and about 40%, on a dry weight basis. In some embodiments a high-protein pea plant or seed has at least 0.25%, 0.5%, 0.75%, 1.0%, 1.5%, 2.0%, 2.5%, 3.0%, 3.5%, 4.0%, 4.5%, 5%, 6%, 7%, or 8% more protein content than a reference pea plant or seed. In certain embodiments the reference pea plant or seed is a commodity pea plant or commodity pea seed.

As used herein, a “population of plants,” “population of seeds,” “plant population,” or “seed population” means a set comprising any number, including one, of individuals, objects, or data from which samples are taken for evaluation, e.g., estimating quantitative trait locus (QTL). Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silico representations of the plants or seeds. The population members need not be identical to the population members selected for use in subsequent cycles of analyses or those ultimately selected to obtain final progeny plants or seeds. Often, a plant or seed population is derived from a single biparental cross, but may also derive from two or more crosses between the same or different parents. Although a population of plants or seeds may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

A “high-protein population” of plants refers to a population of plants having greater seed protein content than a reference sample population of the same plant species. In specific embodiments, a high-protein pea population or a high-protein population of pea plants has a seed protein content of at least 20%, 21%, 22%, 23%, 24%, 25%, 26%, 27%, 28%, 29%, or 30% by weight. In particular embodiments a high protein population comprises a seed protein content of at least 26.5%, 27%, 27.5%, or 28% by weight. In specific embodiments, a high-protein population of peas (i.e., pea seeds) has greater seed protein content than a population of commodity pea seeds. A population of commodity peas may have a protein content of less than 26.5%, or between about 20% and about 26.5%, on a dry weight basis. In some embodiments, a population high-protein pea plants or seeds has at least 0.25%, 0.5%, 0.75%, 1.0%, 1.5%, 2.0%, 2.5%, 3.0%, 3.5%, 4.0%, 4.5%, 5%, 6%, 7%, or 8% more protein content than a reference population of pea plants or seeds. In certain embodiments the reference population of pea plants or seeds is a population of commodity pea plants or commodity pea seeds.

As used herein, the term “crop performance” is used synonymously with “plant performance” and refers to of how well a plant grows under a set of environmental conditions and cultivation practices. Crop performance can be measured by any metric a user associates with a crop's productivity (e.g., yield), appearance and/or robustness (e.g., color, morphology, height, biomass, maturation rate, etc.), product quality (e.g., fiber lint percent, fiber quality, seed protein content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.).

Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

A “microbe” will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers. In various embodiments, a microbe can be any of several different microscopic stages of a plant or animal. Microbes also include viruses, viroids, and prions, especially those which are pathogens or symbionts to crop plants. A “pathogen” as used herein refers to a microbe that causes disease or harmful effects on plant health.

A “fungus” includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus.

A “pest” is any organism that can affect the performance of a plant in an undesirable way. Common pests include microbes, animals (e.g. insects and other herbivores), and/or plants (e.g. weeds). Thus, a pesticide is any substance that reduces the survivability and/or reproduction of a pest, e.g. fungicides, bactericides, insecticides, herbicides, and other toxins.

“Tolerance” or “improved tolerance” in a plant to disease conditions (e.g. growing in the presence of a pest) will be understood to mean an indication that the plant is less affected by the presence of pests and/or disease conditions with respect to yield, survivability and/or other relevant agronomic measures, compared to a less tolerant, more “susceptible” plant. Tolerance is a relative term, indicating that a “tolerant” plant survives and/or performs better in the presence of pests and/or disease conditions compared to other (less tolerant) plants (e.g., a different pea cultivar) grown in similar circumstances. As used in the art, “tolerance” is sometimes used interchangeably with “resistance,” although resistance is sometimes used to indicate that a plant appears maximally tolerant to, or unaffected by, the presence of disease conditions. Plant breeders of ordinary skill in the art will appreciate that plant tolerance levels vary widely, often representing a spectrum of more-tolerant or less-tolerant phenotypes, and are thus trained to determine the relative tolerance of different plants, plant lines or plant families and recognize the phenotypic gradations of tolerance.

“Yield” as used herein is defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance, photosynthetic carbon assimilation rates, and early vigor may also be important factors in determining yield. Optimizing the abovementioned factors may therefore contribute to increasing crop yield. Yield can be measured and expressed by any means known in the art. In specific embodiments, yield is measured by seed weight or volume in a given harvest area.

As used herein, “yield penalty” refers to a reduction of seed yield in a line correlated with or caused by the presence of a high-protein allele or genotype as compared to a line that does not contain that high-protein allele or genotype. In some embodiments, a yield penalty can be a partial yield penalty, such as a reduction of yield by about 0.5%, 1.0%, 1.5%, 2.0%, 2.5%, 3.0%, 3.5%, 4.0%, 4.5%, or about 5.0%, 6%, 7%, 8%, 9%, or about a 10% reduction in yield when compared to a pea variety that does not contain the high-protein allele or deletion. In specific embodiments, the yield penalty is about a 0-5%, 0.5-4.5%, 0.5-4%, 1-5%, 1-4%, 2-5%, 2-4%, 0.5-10%, 0.5-8%, 1-10%, 2-10%, 3-10%, 4-10%, 5-10%, 6-10%, 7-10%, or about an 8-10% reduction in yield when compared to a pea variety that does not contain the high-protein allele or deletion.

As used herein, “selecting” or “selection” in the context of marker-assisted selection or breeding refer to the act of picking or choosing desired individuals, normally from a population, based on certain pre-determined criteria.

As used herein the term “polynucleotide” refers to a single or double stranded nucleic acid sequence which is isolated and provided in the form of an RNA sequence (e.g., an mRNA sequence), a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In certain embodiments, a user can combine the teachings herein with high-density molecular marker profiles spanning substantially the entire genome of a plant to estimate the value of selecting certain candidates in a breeding program in a process commonly known as genome selection.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

### 2.1 Methods of Producing High-Protein Pea Plants or Seeds

In an aspect, this disclosure provides a method of creating a population of high-protein pea plants or seeds. The method comprises the steps of: (a) genotyping a first population of pea plants or seeds for the presence of at least one high-protein molecular marker that is within 20 centimorgans of one or more high protein Quantitative Trait Locus (QTLs) selected from the group consisting of Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps02_432513197, Ps02_440456554, Ps03_158264810, Ps03_205819517, Ps03_206829164, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps06_410567663, Ps06_427519500, Ps06_446483044, Ps07_9801763, Ps07_20773355, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897; b) selecting from the first population one or more pea plants or seeds comprising one or more high-protein alleles having the one or more high-protein molecular markers; and c) producing a second population of progeny pea plants or seeds from the selected one or more pea plants or plants grown from the selected seeds, wherein the second population of progeny pea plants or seeds comprises the one or more high-protein alleles having the one or more high-protein molecular markers, and wherein the second population of progeny pea plants or seeds are high-protein pea plants or seeds, thereby producing a population of high-protein pea plants or seeds.

The above-described high protein QTLs are further described in Table 1. Each QTL (such as “Ps01_20222535”) comprises the SNP marker associated with high protein content as indicated in Table 1. The genomic sequence upstream and downstream of the SNP marker of the high protein QTL can include a nucleic acid sequence that has at least 90% sequence identity with the nucleic acid sequence described in Table 1. Each marker listed in Table 1 (referred to as “marker AA”) can also refer to an SNP marker at position 101 of the nucleic acid sequence identified for the marker AA (SEQ ID NO: BB) in a genomic region comprising the nucleic acid sequence of SEQ ID NO: BB, or at a corresponding position of a genomic region at least about 50, 100, 150, or 200 nucleotides of which is aligned to SEQ ID NO: BB for maximum homology (e.g., 90%, 91%, 92%, 93%, 94%, 95%, 96%, 97%, 98%, or 99% homology) when using standard alignment parameters.

For example, the QTL identified as Ps05_49389403 or chr5-1 can have a sequence that has at least 90% identity with SEQ ID NO: 148. The marker identified as Ps05_49389403 or chr5-1 can also refer to a SNP marker at position 101 of a nucleic acid sequence of SEQ ID NO: 148 in a genomic region comprising the nucleic acid sequence of SEQ ID NO: 148, or at a corresponding position of a genomic region at least about 50, 100, 150, or 200 nucleotides which is aligned to SEQ ID NO: 148 for maximum homology (e.g., 90%, 91%, 92%, 93%, 94%, 95%, 96%, 97%, 98%, or 99% homology) when using standard alignment parameters.

In some embodiments, at least one high protein molecular marker is within 0.5, 1, 1.5, 2, 2.5,, 3, 3.5, 4, 4.5, 5, 5.5, 6. 6.5, 7, 7.5, 8, 8.5, 9, 9.5, or 10 centimorgans of said one or more high protein QTLs.

In one embodiment of the method, the high protein QTL is Ps03_531239107 and/or Ps05_49389403. In another embodiment, the high protein QTL is Ps03_531014546, Ps03_531232613, and/or Ps03_531239107.

In some embodiments, selecting from the first population one or more pea plants or seeds is based on detection of the presence of a high-protein haplotype. A high protein haplotype can comprise high-protein alleles of two or more polymorphic loci described herein.

Provided herein are methods of producing a population of high-protein pea plants or seeds having a high-protein phenotype. In specific embodiments, the high-protein pea plants or seeds combine high-protein content without a corresponding reduction or penalty in crop yield. Methods of producing a population of high-protein pea plants or seeds combining commercially significant yield and high protein content without a corresponding reduction in seed oil are disclosed herein. In some embodiments, methods of producing a population of high-protein pea plants or seeds with a mean whole seed total protein content of greater than 26.5%, 27%, 27.5%, or 28% are provided. In some embodiments, the disclosure provides methods of producing a population of high-protein pea plants or seeds with a mean whole seed total protein content of greater than 26.5%, 27%, 27.5%, or 28% The plants described in embodiments herein may have, for example, a yield in excess of 48 bushels per acre.

The mean seed protein content of the high-protein pea plants and seeds disclosed herein have a protein content of at least 20%, 21%, 22%, 23%, 24%, 25%, 26%, 27%, 28%, 29%, or 30% protein by weight. In specific embodiments, the mean whole seed total protein content is between 20% and 30%, 20% and 24%, 22% and 26%, 24% and 26%, 26% and 28%, 24% and 30%, or 26% and up to about 30%. In further embodiments of the invention, the mean whole seed total protein content at least 26.5% and up to 30%. In certain embodiments, the mean seed protein content of the plants of the invention may further comprise a mean whole seed total protein of at least 26%, at least 26.5%, at least 27%, or at least 27.5%, and the mean yield that is in excess of 48 bushels per acre.

QTLs (i.e., high protein QTLs) that exhibit significant co-segregation with high protein phenotype are provided herein. In specific embodiments, plants or seeds comprising the high-protein QTLs further comprise one or more allele associated with high yield. In some embodiments, the one or more allele associated with high yield is within 10 centimorgans or less, e.g., 9.5 centimorgans or less, 9 centimorgans or less, 8.5 centimorgans or less, 8 centimorgans or less, 7.5 centimorgans or less, 7 centimorgans or less, 6.5 centimorgans or less, 6 centimorgans or less, 5.5 centimorgans or less, 5 centimorgans or less, 4.5 centimorgans or less, 4 centimorgans or less, 3.5 centimorgans or less, 3 centimorgans or less, 2.5 centimorgans or less, 2 centimorgans or less, 1.5 centimorgans or less, 1 centimorgans or less, or 0.5 centimorgans or less from one or more high yield QTLs. High-protein QTLs can be tracked during plant breeding or introgressed into a desired genetic background in order to provide plants exhibiting high protein and, in specific embodiments, one or more other beneficial traits. In an aspect, this disclosure identifies QTL intervals that are associated with high protein in different pea varieties described herein.

In specific embodiments, high-protein molecular markers are associated with a plants or plant parts having a higher protein content than corresponding plants or plant parts without the high-protein molecular marker. The higher protein content in plants and plant parts having at least one high-protein molecular marker (e.g., SNP or deletion marker) disclosed herein can be at least about 0.5%, 0.6%, 0.7%, 0.8%, 0.9%, 1.0%, 1.05%, 1.1%, 1.11%, 1.12%, 1.13%, 1.14%, 1.15%, 1.16%, 1.17%, 1.18%, 1.19%, 1.2%, 1.3%, 1.4%, 1.5%, 1.6%, 1.7%, 1.8%, 1.9%, or about 2.0%, 2.5%, 3.0%, 3.5%, or 4% greater than corresponding plants or plant parts without the high-protein molecular marker.

High protein markers of the present disclosure include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

High protein markers, such as simple sequence repeat markers (SSR), AFLP markers, RFLP markers, RAPD markers, phenotypic markers, single nucleotide polymorphisms (SNPs), isozyme markers, deletion markers, microarray transcription profiles that are genetically linked to or correlated with alleles of a QTL of the present invention can be utilized (Walton, Seed World 22-29 (July 1993), Burow et al., Molecular Dissection of Complex Traits, 13-29, ed. Paterson, CRC Press, New York (1988)). Methods to isolate and identify such markers are known in the art. For example, locus-specific SSR markers can be obtained by screening a genomic library for microsatellite repeats, sequencing of “positive” clones, designing primers which flank the repeats, and amplifying genomic DNA with these primers. The size of the resulting amplification products can vary by integral numbers of the basic repeat unit. To detect a polymorphism, PCR products can be radiolabeled, separated on denaturing polyacrylamide gels, and detected by autoradiography. Fragments with size differences >4 bp can also be resolved on agarose gels, thus avoiding radioactivity.

SNPs occur at a single nucleotide. SNPs are more stable than other classes of polymorphisms. Their spontaneous mutation rate is approximately 10−9 (Kornberg, DNA Replication, W. H. Freeman & Co., San Francisco (1980)). As SNPs result from sequence variation, new polymorphisms can be identified by sequencing random genomic or cDNA molecules. SNPs can also result from deletions, point mutations and insertions. That said, SNPs are also advantageous as markers since they are often diagnostic of “identity by descent” because they rarely arise from independent origins. Any single base alteration, whatever the cause, can be a SNP. SNPs occur at a greater frequency than other classes of polymorphisms and can be more readily identified. In the present disclosure, a SNP can represent a single indel event, which may consist of one or more base pairs, or a single nucleotide polymorphism.

A marker (e.g., an SNP marker) associated with protein content can be a positive marker or a negative marker. A “positive marker” as used herein refers to a marker in which the allele has a positive effect on protein content. A “negative marker” as used herein refers to a marker in which the allele has a negative effect on protein content. A “high-protein marker” (e.g., a “high-protein SNP marker”) as used herein refers to a positive marker, e.g., an allele associated with high protein content. As used herein, a “reference allele” refers to one variation of the SNP sequence (e.g., a nucleotide), and an “alternate allele” refers to another variation of the SNP sequence (e.g., a nucleotide). Alleles can also be referred to as a “major allele” (referring to the most common (or frequent) variation of a sequence (e.g., a nucleotide)), and a “minor allele” (referring to a less common (or frequent) variation of a sequence (e.g., a nucleotide). Example reference and alternate alleles for high-protein markers are set forth for instance in Table 1. Table 1 sets forth example high-protein markers with marker weight, as expressed by Lasso protein coefficient. A “marker weight” as used herein, expressed in some embodiments as a Lasso protein coefficient, refers to the significance of association of the marker with the high protein content, wherein a “positive marker weight” indicates that the alternate allele has a positive effect on protein content (i.e., the alternate allele is a positive marker and the reference allele is a negative marker), and a “negative marker weight” indicates that the alternate allele has a negative effect on protein content (i.e., the alternate allele is a negative marker and the reference allele is a positive marker). In some embodiments, a marker weight greater than a cut-off value or less than a cut-off value indicates a significant association of the marker with high protein content. The cut-off value can be determined by one skilled in the art. For example, a marker weight greater than 0.01 or less than 0.01; greater than 0.02 or less than 0.02; greater than 0.025 or less than 0.025; greater than 0.03 or less than 0.03; greater than 0.04 or less than 0.04; greater than 0.05 or less than 0.05; or greater than 0.1 or less than 0.1. Table 1 includes QTLs having greater than 0.025 or less than 0.025 marker weight (LASSO protein coefficient), with a positive LASSO protein coefficient value indicating that the alternate allele is associated with increased protein content, and a negative LASSO protein coefficient value indicating that the reference allele is associated with increased protein content. For example, in some embodiments, high protein SNP markers Ps05_48702172, Ps05_268032915, and Ps05_358014672 (and others listed in Table 1) have a positive marker weight, with the alternate allele associated with high protein content. Ps03_531239107 (chr3-4) is also a high protein SNP marker with a positive marker weight, the alternate allele (a C) being associated with the high protein content. On the other hand, high protein SNP markers Ps04_26115694, Ps05_500234888, Ps04_464099084, Ps05_217776534, and Ps05_139126831 (and others listed in Table 1) have a negative marker weight, with the reference allele associated with high protein content. Ps05_49389403 (chr5-1) is also a high protein SNP marker with a negative marker weight, the reference allele (an A) being associated with high protein content.

In some embodiments, high protein markers with positive marker weight include Ps03_531239107, Ps01_113369982, Ps01_20222535, Ps01_264925535, Ps01_280789385, Ps01_300614888, Ps01_324252121, Ps01_349096914, Ps01_436651445, Ps01_55991509, Ps01_88206114, Ps01_95579585, Ps02_296256342, Ps02_313767424, Ps02_389051201, Ps03_158264810, Ps03_205819517, Ps03_238101773, Ps03_241025997, Ps03_483314788, Ps03_513771826, Ps03_531014546, Ps03_531232613, Ps04_119030031, Ps04_126746363, Ps04_198084088, Ps04_198169869, Ps04_256098157, Ps04_263312773, Ps04_327258970, Ps04_347955117, Ps04_374415380, Ps04_378090615, Ps04_434414625, Ps04_445153308, Ps04_457675265, Ps04_467088335, Ps05_175640373, Ps05_23320549, Ps05_265627777, Ps05_268032915, Ps05_284520856, Ps05_289702502, Ps05_320108884, Ps05_338232797, Ps05_352490739, Ps05_358014672, Ps05_456631333, Ps05_48702172, Ps05_5262178, Ps05_534247077, Ps05_543517276, Ps05_54722636, Ps05_550603121, Ps05_576520275, Ps06_108179595, Ps06_137271101, Ps06_261243645, Ps06_383667570, Ps06_410567663, Ps06_71058460, Ps06_75832558, Ps06_79052113, Ps06_91302660, Ps06_97595572, Ps07_112377551, Ps07_173321635, Ps07_231299734, Ps07_241735133, Ps07_337883272, Ps07_466821729, Ps07_482615897, Ps07_57031312, and Ps07_89781713.

In some embodiments, high protein markers with negative marker weight include Ps05_49389403, Ps01_113369984, Ps01_120406755, Ps01_160458316, Ps01_22514126, Ps01_279286967, Ps01_367706416, Ps01_380906255, Ps01_440892085, Ps01_78756169, Ps01_87632539, Ps02_149117835, Ps02_162193050, Ps02_17543117, Ps02_249953551, Ps02_282186543, Ps02_293278647, Ps02_298578096, Ps02_432513197, Ps02_440456554, Ps02_64161520, Ps03_206829164, Ps03_481796573, Ps03_507346266, Ps03_511404191, Ps04_106176050, Ps04_133748675, Ps04_140768543, Ps04_196413843, Ps04_26115694, Ps04_284358817, Ps04_386293806, Ps04_463538432, Ps04_464099084, Ps04_9648139, Ps05_134772954, Ps05_139126831, Ps05_17115394, Ps05_173144250, Ps05_217776534, Ps05_277838646, Ps05_290623435, Ps05_337541850, Ps05_409869744, Ps05_500234888, Ps05_51336818, Ps05_53552642, Ps05_551582581, Ps05_556990553, Ps05_564305756, Ps05_568744565, Ps05_591946858, Ps05_596172019, Ps06_1859845, Ps06_32259152, Ps06_375201129, Ps06_402503684, Ps06_427519500, Ps06_446483044, Ps07_131261098, Ps07_155895151, Ps07_20773355, Ps07_235684752, Ps07_238767894, Ps07_274069066, Ps07_314769485, Ps07_327087818, Ps07_466233654, Ps07_46743665, Ps07_50335973, Ps07_55350864, Ps07_58281807, Ps07_84885129, and Ps07_9801763.

An “anchor marker” as used herein refers to a SNP marker that has a significant association with high protein content, and includes a positive marker and a negative marker. Each anchor marker can have one or more neighboring markers (SNP markers), also referred to as “satellite” markers (SNP markers). The distance between the anchor marker and the satellite marker can be any distance, for example 0.001 centimorgan to 10 centimorgan, e.g., about 0.001-0.01, 0.01-1, or 1-10 centimorgan. One or more satellite markers can be used to increase the distance (e.g., centimorgan) from the anchor marker within which the anchor marker can exert its association with high protein phenotype, or can accurately predict a high-protein plant. For example, the methods of producing a population of high-protein pea plants or seeds provided herein can comprise genotyping a first population of pea plants or seeds for the presence of at least one high-protein anchor marker that is within a certain distance from the high-protein QTL, e.g., 10 centimorgans (e.g., 0.5, 1, 1.5, 2, 2.5, 3, 3.5, 4, 4.5, 5, 5.5, 6, 6.5, 7, 7.5, 8, 8.5, 9, 9.5, 10) from the high-protein QTL, or the presence of at least one satellite marker associated with the anchor marker that is within a longer distance from the high-protein QTL, e.g., 20 centimorgans (e.g., 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20) from the high-protein QTL. Similarly, the methods of introgressing a high protein QTL provided herein can comprise selecting a progeny plant or seed comprising a high-protein allele of a polymorphic locus linked to the high-protein QTL, wherein the polymorphic locus can be an anchor marker that is within a certain distance from the high-protein QTL, e.g., 10 centimorgans (e.g., 0.5, 1, 1.5, 2, 2.5, 3, 3.5, 4, 4.5, 5, 5.5, 6, 6.5, 7, 7.5, 8, 8.5, 9, 9.5, 10) from the high-protein QTL, or the polymorphic locus can be a satellite marker associated with the anchor marker that is within a longer distance from the high-protein QTL, e.g., 20 centimorgans (e.g., 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20) from the high-protein QTL.

In some embodiments an SNP marker at high-protein QTL Ps03_531239107 comprises a C at position 425968088 of chromosome 3 of the Pisum sativum genome; a C at position 563531992 of chromosome 5; or a C at position 101 of SEQ ID NO: 47 in a genomic region comprising the nucleic acid sequence of SEQ ID NO: 47, or at a corresponding position of a genomic region at least about 50, 100, 150, or 200 nucleotides of which is aligned to SEQ ID NO: 47 for maximum homology (e.g., 90%, 91%, 92%, 93%, 94%, 95%, 96%, 97%, 98%, or 99% homology) when using standard alignment parameters. In some embodiments an SNP marker at high-protein QTL Ps05_49389403 comprises an A at position 36835261 of chromosome 5; or an A at position 101 of SEQ ID NO: 148 in a genomic region comprising the nucleic acid sequence of SEQ ID NO: 148, or at a corresponding position of a genomic region at least about 50, 100, 150, or 200 nucleotides of which is aligned to SEQ ID NO: 148 for maximum homology (e.g., 90%, 91%, 92%, 93%, 94%, 95%, 96%, 97%, 98%, or 99% homology) when using standard alignment parameters. In some embodiments an SNP marker at high-protein QTL comprises the SNP at the positions described in Table 1, with the alternate allele associated with high protein content for markers when the protein Lasso value is positive, and the reference allele associated with high protein content for markers when the protein Lasso value is negative. For example, an SNP marker at high-protein QTL comprises a C at position 425968088 of chromosome 3; a C at position 563531992 of chromosome 5; a C at position 101 of SEQ ID NO: 47 in a genomic region comprising the nucleic acid sequence of SEQ ID NO: 47, or at a corresponding position of a genomic region at least 50 nucleotides of which is aligned to SEQ ID NO: 47 for at least 90% sequence identity; an A at position 36835261 of chromosome 5; an A at position 101 of SEQ ID NO: 148 in a genomic region comprising the nucleic acid sequence of SEQ ID NO: 148, or at a corresponding position of a genomic region at least 50 nucleotides of which is aligned to SEQ ID NO: 148 for at least 90% sequence identity; a G at position 36095 of scaffold 04655; a T at position 108299170 of chromosome 1LG6; a G at position 157869 of scaffold 00066; a T at position 23108565 of chromosome 1LG6; an A at position 122338117 of chromosome 1LG6; a G at position 306857129 of chromosome 1LG6; a G at position 45686305 of chromosome 1LG6; an A at position 371072249 of chromosome 1LG6; a G at position 72083191 of chromosome 1LG6; a C at position 79225752 of chromosome 1LG6; a T at position 8290 of scaffold 02021; an A at position 119392808 of chromosome 2LG1; a T at position 10842575 of chromosome 2LG1; an A at position 169314375 of chromosome 2LG1; a G at position 286755665 of chromosome 2LG1; a G at position 91532 of scaffold 00644; a C at position 301660243 of chromosome 2LG1; an A at position 420361771 of chromosome 2LG1; a G at position 426699364 of chromosome 2LG1; a C at position 26206979 of chromosome 2LG1; a G at position 30219372 of chromosome 3LG5; an A at position 393751811 of chromosome 3LG5; an A at position 417958980 of chromosome 3LG5; a G at position 421049387 of chromosome 3LG5; a T at position 83578489 of chromosome 4LG4; a C at position 109277412 of chromosome 4LG4; an A at position 117043426 of chromosome 4LG4; a G at position 163719335 of chromosome 4LG4; a T at position 18486554 of chromosome 4LG4; a C at position 247901046 of chromosome 4LG4; a T at position 2191851 of chromosome 4LG4; a C at position 444278355 of chromosome 4LG4; a T at position 445125850 of chromosome 4LG4; a T at position 5972665 of chromosome 4LG4; an A at position 96025751 of chromosome 5LG3; an A at position 12104 of scaffold 00462; a C at position 178039871 of chromosome 5LG3; an A at position 132883215 of chromosome 5LG3; a G at position 24130766 of chromosome 5LG3; a T at position 228797264 of chromosome 5LG3; a G at position 239060496 of chromosome 5LG3; a C at position 2288318 of chromosome 5LG3; a G at position 331834371 of chromosome 5LG3; a T at position 50774 of scaffold 02833; a C at position 37349400 of chromosome 5LG3; a G at position 39703 of super-scaffold 888; a G at position 509926370 of chromosome 5LG3; a C at position 509729669 of chromosome 5; an A at position 522716439 of chromosome 5LG3; a T at position 124873928 of chromosome 5LG3; a G at position 551226342 of chromosome 5LG3; an A at position 547326524 of chromosome 5; a T at position 1621846 of chromosome 6LG2; an A at position 4002 of scaffold 00839; a T at position 374758162 of chromosome 6LG2; a T at position 401325650 of chromosome 6LG2; a C at position 426328393 of chromosome 6LG2; a G at position 438943398 of chromosome 6; a T at position 72341 of scaffold 02959; a G at position 89032441 of chromosome 7LG7; an A at position 19382049 of chromosome 7LG7; a C at position 310437720 of chromosome 7LG7; a T at position 310515874 of chromosome 7LG7; a C at position 335690162 of chromosome 7LG7; an A at position 322450055 of chromosome 7; a G at position 10989 of scaffold 00840; an A at position 460750292 of chromosome 7LG7; a G at position 13304 of scaffold 06512; a T at position 52311972 of chromosome 7; a G at position 50802012 of chromosome 7LG7; an A at position 56383957 of chromosome 7LG7; a G at position 1311773 of chromosome 7LG7; a G at position 8316015 of chromosome 7LG7; a C at position 36097 of scaffold 04655; an A at position 20877277 of chromosome 1LG6; a T at position 194893633 of chromosome 1LG6; a G at position 72152 of scaffold 03789; an A at position 30542636 of chromosome 1LG6; a T at position 116776833 of chromosome 1LG6; an A at position 288453266 of chromosome 1LG6; a G at position 367968951 of chromosome 1LG6; a T at position 51330566 of chromosome 1LG6; a C at position 29379 of scaffold 02116; a G at position 87185358 of chromosome 1LG6; a G at position 5787797 of chromosome 2LG1; a C at position 87090294 of chromosome 2LG1; an A at position 383268619 of chromosome 2LG1; a C at position 104891818 of chromosome 3LG5; a G at position 72342 of scaffold 00254; a T at position 173063548 of chromosome 3LG5; a T at position 174636272 of chromosome 3LG5; a C at position 396332351 of chromosome 3LG5; a G at position 423551062 of chromosome 3LG5; a C at position 827484 of chromosome 3LG5; an A at position 425962517 of chromosome 3; a T at position 94928513 of chromosome 4LG4; an A at position 47049 of scaffold 02127; a C at position 165487268 of chromosome 4LG4; an A at position 165597701 of chromosome 4LG4; an A at position 218884465 of chromosome 4LG4; a G at position 228522691 of chromosome 4LG4; a C at position 352524054 of chromosome 4LG4; an A at position 363782042 of chromosome 4LG4; a G at position 285377934 of chromosome 4LG4; a G at position 389689436 of chromosome 4LG4; an A at position 145804 of scaffold 00706; an A at position 374997960 of chromosome 4LG4; a G at position 418184353 of chromosome 4LG4; an A at position 420833970 of chromosome 4LG4; an A at position 134409547 of chromosome 5LG3; an A at position 20543180 of chromosome 5LG3; a G at position 217510948 of chromosome 5LG3; a C at position 84199 of scaffold 02449; a G at position 234213508 of chromosome 5LG3; a T at position 236504935 of chromosome 5LG3; a C at position 268153046 of chromosome 5LG3; a T at position 278088295 of chromosome 5LG3; an A at position 84459019 of chromosome 5LG3; a G at position 306598116 of chromosome 5LG3; a G at position 413429268 of chromosome 5; a G at position 35018191 of chromosome 5LG3; a C at position 362278 of chromosome 5LG3; a C at position 492763269 of chromosome 5LG3; a G at position 499899891 of chromosome 5LG3; a T at position 39908055 of chromosome 5LG3; a T at position 143390359 of chromosome 5LG3; a G at position 535824046 of chromosome 5LG3; an A at position 89382481 of chromosome 6LG2; a T at position 111705293 of chromosome 6LG2; an A at position 191916418 of chromosome 6LG2; a G at position 303558968 of chromosome 6LG2; a T at position 406586297 of chromosome 6LG2; a C at position 17855 of scaffold 05469; an A at position 62010766 of chromosome 6LG2; a T at position 64688438 of chromosome 6LG2; a C at position 75309486 of chromosome 6LG2; a T at position 86301013 of chromosome 6LG2; an A at position 45871271 of chromosome 7LG7; an A at position 16557044 of chromosome 7LG7; a T at position 223304507 of chromosome 7LG7; an A at position 158981077 of chromosome 7LG7; a C at position 365136400 of chromosome 7LG7; a G at position 47207 of scaffold 01757; an A at position 481276628 of chromosome 7LG7; an A at position 52153701 of chromosome 7LG7; or a G at position 88161594 of chromosome 7LG7 of a Pisum sativum genome. In specific embodiments, the Pisum sativum genome referred to herein is the Cameor v1a reference genome.

In specific embodiment, the high-protein QTL comprises a deletion marker. As used herein, a “deletion marker” refers to a deletion of a nucleotide region in the genome of plants or plant parts exhibiting a high-protein phenotype. Plants or plant parts having genomes lacking the deletion marker exhibit a lower protein content by weight than the plants and plant parts having genomes with the deletion marker. The deleted nucleotide region of a deletion marker can be a deletion of any number of consecutive nucleotides that is associated with a high-protein phenotype. For example, the deletion can be 2-500 bp, 5-250 bp, 10-200 bp, 20-180 bp, 40-160 bp, 50-140 bp, 60-120 bp, 70-100 bp, 80-100 bp, 85-95 bp, or about 2 bp, 5 bp, 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 100 bp, 105 bp, 110 bp, 120 bp, 130 bp, 140 bp, 150 bp, 160 bp, 170 bp, 180 bp, 200 bp, 225 bp, 250 bp, 275 bp, 300 bp, 350 bp, 400 bp, 450 bp, or about 500 bp. In certain embodiments, the deletion marker is 87 bp, 88 bp, or 89 bp.

In specific embodiments, the deletion maker can be wholly or at least partially within a gene. The deletion marker can be wholly or at least partially within an exon or intron of the gene. That is, the deletion marker can be a deletion of a nucleotide sequence entirely within a gene or spanning the 5′ end of the gene or the 3′ of the gene. In some embodiments, the deletion marker eliminates the start codon of a gene. The deletion marker can also account for removal of a signal peptide of a gene. In some embodiments, the deletion marker eliminates both the start codon and the signal peptide of a gene. The gene can be any gene in the genome.

The high-protein QTLs disclosed herein can be an expression QTL (eQTL). As used herein an eQTL refers to a QTL that is associated with differential expression of a gene. In specific embodiments, when a QTL is present in the genome, a gene associated with the eQTL is has reduced expression. For example, the presence of an eQTL can eliminate or substantially elimination expression of a gene.

As disclosed herein, a pea plant or seed refers to a plant, plant part, or seed of Pisum sativum. In specific embodiments, all chromosomal positions listed herein are identified relative to the reference genome, such as the Cameor v1a reference genome. The wild perennial peas belong to the genus Pisum and have a wide array of genetic diversity. In some embodiments described herein, the pea plant or seed is a members of the genus Pisum, such as Pisum sativum and Pisum fulvum. In specific embodiments, the plants, plant parts, or plant products comprise at least one high-protein QTL disclosed herein. For example, in specific embodiments, a pea seed or pea protein product (e.g., pea protein concentrate, pea protein, or pea protein isolate) comprise at least one marker selected from Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps02_432513197, Ps02_440456554, Ps03_158264810, Ps03_205819517, Ps03_206829164, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps06_410567663, Ps06_427519500, Ps06_446483044, Ps07_9801763, Ps07_20773355, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897.

### 2.2 Methods of Introgressing a High-Protein QTL

Provided herein are methods for selection and introgression of a high-protein QTL. The methods comprise the steps of (a) crossing a first pea plant comprising a high-protein QTL with a second pea plant of a different genotype to produce one or more progeny plants or seeds; and (b) selecting a progeny plant or seed comprising a high-protein allele of a polymorphic locus linked to the high-protein QTL. The polymorphic locus described herein is a chromosomal segment comprising any marker within the genomic regions 421,829,254-437,541,609 of chromosome 3, 1-54716217 of chromosome 5, 20877277-371072249 of chromosome 1LG6, 10842575-426699364 of chromosome 2LG1, 104891818-425968089 of chromosome 3LG5, 5972665-445125850 of chromosome 4LG4, 362278-547326524 of chromosome 5LG3, 1621846-438943399 of chromosome 6LG2, 8316015-481276628 of chromosome 7LG7, scaffold 02116, scaffold 04655, scaffold 00066, scaffold 03789, scaffold 02021, scaffold 00644, scaffold 00254, scaffold 02127, scaffold 00706, super-scaffold 888, scaffold 00462, scaffold 02449, scaffold 02833, scaffold 00839, scaffold 05469,, scaffold 06512, scaffold 02959 scaffold 00840, or scaffold 01757 of a Pisum sativum genome. In specific embodiments, the Pisum sativum genome is the Cameor v1a reference genome.

In some embodiments, selecting the progeny plant or seed from the population is based on the presence of a high-protein haplotype. In particular embodiments, a high protein haplotype comprises alleles of two or more polymorphic loci described herein.

In a specific embodiment of the method, the high-protein QTL comprises at least one SNP that is within the genomic region 421829254-437541609 of chromosome 3. In a specific embodiment, the high-protein QTL comprises at least one SNP that is within the genomic region 1-54716217 of chromosome 5.

In some embodiments of the method of introgressing a high-protein QTL, the high protein SNP is selected from the group consisting of: a C at position 425968088 of chromosome 3; a C at position 563531992 of chromosome 5; a C at position 101 of SEQ ID NO: 47 in a genomic region comprising the nucleic acid sequence of SEQ ID NO: 47, or at a corresponding position of a genomic region at least 50 nucleotides of which is aligned to SEQ ID NO: 47 for at least 90% sequence identity; an A at position 36835261 of chromosome 5; an A at position 101 of SEQ ID NO: 148 in a genomic region comprising the nucleic acid sequence of SEQ ID NO: 148, or at a corresponding position of a genomic region at least 50 nucleotides of which is aligned to SEQ ID NO: 148 for at least 90% sequence identity; a G at position 36095 of scaffold 04655; a T at position 108299170 of chromosome 1LG6; a G at position 157869 of scaffold 00066; a T at position 23108565 of chromosome 1LG6; an A at position 122338117 of chromosome 1LG6; a G at position 306857129 of chromosome 1LG6; a G at position 45686305 of chromosome 1LG6; an A at position 371072249 of chromosome 1LG6; a G at position 72083191 of chromosome 1LG6; a C at position 79225752 of chromosome 1LG6; a T at position 8290 of scaffold 02021; an A at position 119392808 of chromosome 2LG1; a T at position 10842575 of chromosome 2LG1; an A at position 169314375 of chromosome 2LG1; a G at position 286755665 of chromosome 2LG1; a G at position 91532 of scaffold 00644; a C at position 301660243 of chromosome 2LG1; an A at position 420361771 of chromosome 2LG1; a G at position 426699364 of chromosome 2LG1; a C at position 26206979 of chromosome 2LG1; a G at position 30219372 of chromosome 3LG5; an A at position 393751811 of chromosome 3LG5; an A at position 417958980 of chromosome 3LG5; a G at position 421049387 of chromosome 3LG5; a T at position 83578489 of chromosome 4LG4; a C at position 109277412 of chromosome 4LG4; an A at position 117043426 of chromosome 4LG4; a G at position 163719335 of chromosome 4LG4; a T at position 18486554 of chromosome 4LG4; a C at position 247901046 of chromosome 4LG4; a T at position 2191851 of chromosome 4LG4; a C at position 444278355 of chromosome 4LG4; a T at position 445125850 of chromosome 4LG4; a T at position 5972665 of chromosome 4LG4; an A at position 96025751 of chromosome 5LG3; an A at position 12104 of scaffold 00462; a C at position 178039871 of chromosome 5LG3; an A at position 132883215 of chromosome 5LG3; a G at position 24130766 of chromosome 5LG3; a T at position 228797264 of chromosome 5LG3; a G at position 239060496 of chromosome 5LG3; a C at position 2288318 of chromosome 5LG3; a G at position 331834371 of chromosome 5LG3; a T at position 50774 of scaffold 02833; a C at position 37349400 of chromosome 5LG3; a G at position 39703 of super-scaffold 888; a G at position 509926370 of chromosome 5LG3; a C at position 509729669 of chromosome 5; an A at position 522716439 of chromosome 5LG3; a T at position 124873928 of chromosome 5LG3; a G at position 551226342 of chromosome 5LG3; an A at position 547326524 of chromosome 5; a T at position 1621846 of chromosome 6LG2; an A at position 4002 of scaffold 00839; a T at position 374758162 of chromosome 6LG2; a T at position 401325650 of chromosome 6LG2; a C at position 426328393 of chromosome 6LG2; a G at position 438943398 of chromosome 6; a T at position 72341 of scaffold 02959; a G at position 89032441 of chromosome 7LG7; an A at position 19382049 of chromosome 7LG7; a C at position 310437720 of chromosome 7LG7; a T at position 310515874 of chromosome 7LG7; a C at position 335690162 of chromosome 7LG7; an A at position 322450055 of chromosome 7; a G at position 10989 of scaffold 00840; an A at position 460750292 of chromosome 7LG7; a G at position 13304 of scaffold 06512; a T at position 52311972 of chromosome 7; a G at position 50802012 of chromosome 7LG7; an A at position 56383957 of chromosome 7LG7; a G at position 1311773 of chromosome 7LG7; a G at position 8316015 of chromosome 7LG7; a C at position 36097 of scaffold 04655; an A at position 20877277 of chromosome 1LG6; a T at position 194893633 of chromosome 1LG6; a G at position 72152 of scaffold 03789; an A at position 30542636 of chromosome 1LG6; a T at position 116776833 of chromosome 1LG6; an A at position 288453266 of chromosome 1LG6; a G at position 367968951 of chromosome 1LG6; a T at position 51330566 of chromosome 1LG6; a C at position 29379 of scaffold 02116; a G at position 87185358 of chromosome 1LG6; a G at position 5787797 of chromosome 2LG1; a C at position 87090294 of chromosome 2LG1; an A at position 383268619 of chromosome 2LG1; a C at position 104891818 of chromosome 3LG5; a G at position 72342 of scaffold 00254; a T at position 173063548 of chromosome 3LG5; a T at position 174636272 of chromosome 3LG5; a C at position 396332351 of chromosome 3LG5; a G at position 423551062 of chromosome 3LG5; a C at position 827484 of chromosome 3LG5; an A at position 425962517 of chromosome 3; a T at position 94928513 of chromosome 4LG4; an A at position 47049 of scaffold 02127; a C at position 165487268 of chromosome 4LG4; an A at position 165597701 of chromosome 4LG4; an A at position 218884465 of chromosome 4LG4; a G at position 228522691 of chromosome 4LG4; a C at position 352524054 of chromosome 4LG4; an A at position 363782042 of chromosome 4LG4; a G at position 285377934 of chromosome 4LG4; a G at position 389689436 of chromosome 4LG4; an A at position 145804 of scaffold 00706; an A at position 374997960 of chromosome 4LG4; a G at position 418184353 of chromosome 4LG4; an A at position 420833970 of chromosome 4LG4; an A at position 134409547 of chromosome 5LG3; an A at position 20543180 of chromosome 5LG3; a G at position 217510948 of chromosome 5LG3; a C at position 84199 of scaffold 02449; a G at position 234213508 of chromosome 5LG3; a T at position 236504935 of chromosome 5LG3; a C at position 268153046 of chromosome 5LG3; a T at position 278088295 of chromosome 5LG3; an A at position 84459019 of chromosome 5LG3; a G at position 306598116 of chromosome 5LG3; a G at position 413429268 of chromosome 5; a G at position 35018191 of chromosome 5LG3; a C at position 362278 of chromosome 5LG3; a C at position 492763269 of chromosome 5LG3; a G at position 499899891 of chromosome 5LG3; a T at position 39908055 of chromosome 5LG3; a T at position 143390359 of chromosome 5LG3; a G at position 535824046 of chromosome 5LG3; an A at position 89382481 of chromosome 6LG2; a T at position 111705293 of chromosome 6LG2; an A at position 191916418 of chromosome 6LG2; a G at position 303558968 of chromosome 6LG2; a T at position 406586297 of chromosome 6LG2; a C at position 17855 of scaffold 05469; an A at position 62010766 of chromosome 6LG2; a T at position 64688438 of chromosome 6LG2; a C at position 75309486 of chromosome 6LG2; a T at position 86301013 of chromosome 6LG2; an A at position 45871271 of chromosome 7LG7; an A at position 16557044 of chromosome 7LG7; a T at position 223304507 of chromosome 7LG7; an A at position 158981077 of chromosome 7LG7; a C at position 365136400 of chromosome 7LG7; a G at position 47207 of scaffold 01757; an A at position 481276628 of chromosome 7LG7; an A at position 52153701 of chromosome 7LG7; and a G at position 88161594 of chromosome 7LG7 of a Pisum sativum genome. The Pisum sativum genome can be the Cameor v1a reference genome.

In another embodiment, this disclosure further provides methods for introgressing multiple high-protein QTLs identified herein to generate a population of high-protein pea plants or seeds. In some embodiment, the high-protein QTLs are selected from the group consisting of Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps02_432513197, Ps02_440456554, Ps03_158264810, Ps03_205819517, Ps03_206829164, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps06_410567663, Ps06_427519500, Ps06_446483044, Ps07_9801763, Ps07_20773355, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897. In some embodiments, provided herein are methods for concurrently introgressing at least one or more, two or more, three or more, five or more, six or more, seven or more, eight or more, nine or more, ten or more, eleven or more, or twelve high-protein QTLs identified herein to generate a population of high-protein pea plants or seeds.

In certain embodiments of the method, the high protein QTL is Ps03_531239107 and/or Ps05_49389403. In one embodiment, the high protein QTL is Ps03_531014546, Ps03_531232613, and/or Ps03_531239107.

In one embodiment, this disclosure provides a method for introgressing an allele of a polymorphic locus conferring a high-protein phenotype. In specific embodiments, the polymorphic locus comprises any marker within the genomic regions 421,829,254-437,541,609 of chromosome 3, 1-54716217 of chromosome 5, 20877277-371072249 of chromosome 1LG6, 10842575-426699364 of chromosome 2LG1, 104891818-425968089 of chromosome 3LG5, 5972665-445125850 of chromosome 4LG4, 362278-547326524 of chromosome 5LG3, 1621846-438943399 of chromosome 6LG2, 8316015-481276628 of chromosome 7LG7, scaffold 02116, scaffold 04655, scaffold 00066, scaffold 03789, scaffold 02021, scaffold 00644, scaffold 00254, scaffold 02127, scaffold 00706, super-scaffold 888, scaffold 00462, scaffold 02449, scaffold 02833, scaffold 00839, scaffold 05469,, scaffold 06512, scaffold 02959 scaffold 00840, or scaffold 01757 of a Pisum sativum genome. The Pisum sativum genome can be the Cameor v1a reference genome. The marker within the polymorphic locus can be an SNP marker or a deletion marker.

In specific embodiments, the high-protein QTL of the present invention may be introduced into an elite Pisum sativum variety. An “elite” variety as used herein refers to a variety of the plant that has one or more desirable traits, such as high yield, high content of protein or other nutrients, improved flavor, or increased tolerance to disease or environmental pressures.

A high-protein population of pea plants is provided that is produced by any method disclosed herein. In specific embodiments, the high-protein population of pea plants comprises a mean seed protein content that is greater than the mean seed protein content of a control sample population. In some embodiments, the high-protein population of pea plants or seeds comprises at least one high-protein QTL selected from the group consisting of Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps02_432513197, Ps02_440456554, Ps03_158264810, Ps03_205819517, Ps03_206829164, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps06_410567663, Ps06_427519500, Ps06_446483044, Ps07_9801763, Ps07_20773355, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897 at a greater frequency than the occurrence of the same high-protein QTL in a population of pea plants or seeds not produced by the methods disclosed herein. In specific embodiments, a population of pea seeds or pea protein product (e.g., pea protein concentrate, pea protein isolate, or pea protein) is provided herein comprising at least one high-protein QTL disclosed herein at a greater frequency than a control pea seed population or pea protein composition. In some embodiments, a control pea plant or pea seed population or pea protein composition is a population produced by methods without assaying for a high-protein molecular marker, such as those high-protein molecular markers disclosed herein. The high protein pea seeds, plants, and protein compositions disclosed herein need contain or be produced from a population of plants that exclusively contain a high-protein molecular marker disclosed herein.

### 2.3 Detection/Identification of High-Protein Markers and QTLs

The detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

In certain embodiments of the method described herein, genotyping comprises assaying a single nucleotide polymorphism (SNP) marker. SNPs can be assayed and characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or by other biochemical interpretation. SNPs can be sequenced using a variation of the chain termination method (Sanger et al., Proc. Natl. Acad. Sci. (U.S.A.) 74:5463-5467 (1977)) in which the use of radioisotopes are replaced with fluorescently-labeled dideoxy nucleotides and subjected to capillary based automated sequencing (U.S. Pat. No. 5,332,666, the entirety of which is herein incorporated by reference; U.S. Pat. No. 5,821,058, the entirety of which is herein incorporated by reference). Automated sequencers are available from, for example, Applied Biosystems, Foster City, Calif. (3730xl DNA Analyzer), Beckman Coulter, Fullerton, Calif. (CEQ™ 8000 Genetic Analysis System) and LI-COR, Inc., Lincoln, Nebr. (4300 DNA Analysis System).

Approaches for analyzing SNPs can be categorized into two groups. The first group is based on primer-extension assays, such as solid-phase minisequencing or pyrosequencing. In the solid-phase minisequencing method, a DNA polymerase is used specifically to extend a primer that anneals immediately adjacent to the variant nucleotide. A single labeled nucleoside triphosphate complementary to the nucleotide at the variant site is used in the extension reaction. Only those sequences that contain the nucleotide at the variant site will be extended by the polymerase. A primer array can be fixed to a solid support wherein each primer is contained in four small wells, each well being used for one of the four nucleoside triphosphates present in DNA. Template DNA or RNA from each test organism is put into each well and allowed to anneal to the primer. The primer is then extended one nucleotide using a polymerase and a labeled di-deoxy nucleotide triphosphate. The completed reaction can be imaged using devices that are capable of detecting the label which can be radioactive or fluorescent. Using this method several different SNPs can be visualized and detected (Syvänen et al., Hum. Mutat. 13: 1-10 (1999)). The pyrosequencing technique is based on an indirect bioluminometric assay of the pyrophosphate (PPi) that is released from each dNTP upon DNA chain elongation. Following Klenow polymerase mediated base incorporation, PPi is released and used as a substrate, together with adenosine 5-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing procedure (Alderborn et al., Genome Res. 10: 1249-1258 (2000)). An example of an instrument designed to detect and interpret the pyrosequencing reaction is available from Biotage, Charlottesville, Va. (PyroMark MD).

Another SNP detection method based on primer-extension assays is commonly referred to as the GOOD assay. The GOOD assay (Sauer et al., Nucleic Acids Res. 28: e100 (2000)) is an allele-specific primer extension protocol that employs MALDI-TOF (matrix-assisted laser desorption/ionization time-of-flight) mass spectrometry. The region of DNA containing a SNP is amplified first by PCR amplification. Residual dNTPs are destroyed using an alkaline phosphatase. Allele-specific products are then generated using a specific primer, a conditioned set of a-S-dNTPs and a-S-ddNTPs and a fresh DNA polymerase in a primer extension reaction. Unmodified DNA is removed by 5′ phosphodiesterase digestion and the modified products are alkylated to increase the detection sensitivity in the mass spectrometric analysis. All steps are carried out in a single vial at the lowest practical sample volume and require no purification. The extended reaction can be given a positive or negative charge and is detected using mass spectrometry (Sauer et al., Nucleic Acids Res. 28: e13 (2000)). An instrument in which the GOOD assay is analyzed is for example, the AUTOFLEX® MALDI-TOF system from Bruker Daltonics (Billerica, Mass.).

In some embodiments of the method described herein, genotyping comprises assaying a deletion marker. Any method known in the art can be used to identify a region of the genome that is missing a given position, including but not limited to PCR, RFLP, probe-based detection methods, and sequencing methods, among others.

In one embodiment of the method described herein, genotyping comprises the use of an oligonucleotide probe. The use of an oligonucleotide probe is based on recognition of heteroduplex DNA molecules and includes oligonucleotide hybridization, TAQ-MAN® assays, molecular beacons, electronic dot blot assays and denaturing high-performance liquid chromatography. Oligonucleotide hybridizations can be performed in mass using micro-arrays (Southern, Trends Genet. 12: 110-115 (1996)). TAQ-MAN® assays, or Real Time PCR, detects the accumulation of a specific PCR product by hybridization and cleavage of a double-labeled fluorogenic probe during the amplification reaction. A TAQ-MAN® assay includes four oligonucleotides, two of which serve as PCR primers and generate a PCR product encompassing the polymorphism to be detected. The other two are allele-specific fluorescence-resonance-energy-transfer (FRET) probes. FRET probes incorporate a fluorophore and a quencher molecule in close proximity so that the fluorescence of the fluorophore is quenched. The signal from a FRET probes is generated by degradation of the FRET oligonucleotide, so that the fluorophore is released from proximity to the quencher, and is thus able to emit light when excited at an appropriate wavelength. In the assay, two FRET probes bearing different fluorescent reporter dyes are used, where a unique dye is incorporated into an oligonucleotide that can anneal with high specificity to only one of the two alleles. Useful reporter dyes include 6-carboxy-4,7,2′,7′-tetrachlorofluorecein (TET), 2′-chloro-7′-phenyl-1,4-dichloro-6-carboxyfluorescein (VIC) and 6-carboxyfluorescein phosphoramidite (FAM). A useful quencher is 6-carboxy-N,N,N′,N′-tetramethylrhodamine (TAMRA). Annealed (but not non-annealed) FRET probes are degraded by TAQ DNA polymerase as the enzyme encounters the 5′ end of the annealed probe, thus releasing the fluorophore from proximity to its quencher. Following the PCR reaction, the fluorescence of each of the two fluorescers, as well as that of the passive reference, is determined fluorometrically. The normalized intensity of fluorescence for each of the two dyes will be proportional to the amounts of each allele initially present in the sample, and thus the genotype of the sample can be inferred. An example of an instrument used to detect the fluorescence signal in TAQ-MAN® assays, or Real Time PCR are the 7500 Real-Time PCR System (Applied Biosystems, Foster City, Calif.).

Molecular beacons are oligonucleotide probes that form a stem-and-loop structure and possess an internally quenched fluorophore. When they bind to complementary targets, they undergo a conformational transition that turns on their fluorescence. These probes recognize their targets with higher specificity than linear probes and can easily discriminate targets that differ from one another by a single nucleotide. The loop portion of the molecule serves as a probe sequence that is complementary to a target nucleic acid. The stem is formed by the annealing of the two complementary arm sequences that are on either side of the probe sequence. A fluorescent moiety is attached to the end of one arm and a nonfluorescent quenching moiety is attached to the end of the other arm. The stem hybrid keeps the fluorophore and the quencher so close to each other that the fluorescence does not occur. When the molecular beacon encounters a target sequence, it forms a probe-target hybrid that is stronger and more stable than the stem hybrid. The probe undergoes spontaneous conformational reorganization that forces the arm sequences apart, separating the fluorophore from the quencher, and permitting the fluorophore to fluoresce (Bonnet et al., 1999). The power of molecular beacons lies in their ability to hybridize only to target sequences that are perfectly complementary to the probe sequence, hence permitting detection of single base differences (Kota et al., Plant Mol. Biol. Rep. 17: 363-370 (1999)). Molecular beacon detection can be performed for example, on the Mx4000® Multiplex Quantitative PCR System from Stratagene (La Jolla, Calif.).

In one embodiment, the SNP marker described in the methods provided herein is capable of being identified by a corresponding nucleic acid molecule that comprises at least 15 nucleotides that include or are immediately adjacent to the SNP. The nucleic acid molecule described above is at least at least 90% (90%, 91%, 92%, 93%, 94%, 95%, 96%, 97%, 98%, 99%, or 100%) identical to a sequence of the same number of consecutive nucleotides in either strand of DNA that include or are immediately adjacent to the SNP. Likewise, the deletion marker disclosed herein is capable of being identified by a corresponding nucleic acid molecule that comprises at least 15 nucleotides that include or are immediately adjacent to the deletion, or by a nucleic acid molecule that only binds to the unique junction formed by the deletion event.

In one embodiment, the disclosure provides an isolated nucleic acid molecule for detecting a high-protein molecular marker in pea DNA. The nucleic acid molecule comprises at least 15 nucleotides that include or are immediately adjacent to the marker, wherein the nucleic acid molecule is at least 90% (91%, 92%, 93%, 94%, 95%, 96%, 97%, 98%, 99%, or 100%) identical to a sequence of the same number of consecutive nucleotides in either strand of DNA that include or are immediately adjacent to the marker.

The electronic dot blot assay uses a semiconductor microchip comprised of an array of microelectrodes covered by an agarose permeation layer containing streptavidin. Biotinylated amplicons are applied to the chip and electrophoresed to selected pads by positive bias direct current, where they remain embedded through interaction with streptavidin in the permeation layer. The DNA at each pad is then hybridized to mixtures of fluorescently labeled allele-specific oligonucleotides. Single base pair mismatched probes can then be preferentially denatured by reversing the charge polarity at individual pads with increasing amperage. The array is imaged using a digital camera and the fluorescence quantified as the amperage is ramped to completion. The fluorescence intensity is then determined by averaging the pixel count values over a region of interest (Gilles et al., Nature Biotech. 17: 365-370 (1999)).

A more recent application based on recognition of heteroduplex DNA molecules uses denaturing high-performance liquid chromatography (DHPLC). This technique represents a highly sensitive and fully automated assay that incorporates a Peltier-cooled 96-well autosampler for high-throughput SNP analysis. It is based on an ion-pair reversed-phase high performance liquid chromatography method. The heart of the assay is a polystyrene-divinylbenzene copolymer, which functions as a stationary phase. The mobile phase is composed of an ion-pairing agent, triethylammonium acetate (TEAA) buffer, which mediates the binding of DNA to the stationary phase, and an organic agent, acetonitrile (ACN), to achieve subsequent separation of the DNA from the column. A linear gradient of CAN allows the separation of fragments based on the presence of heteroduplexes. DHPLC thus identifies mutations and polymorphisms that cause heteroduplex formation between mismatched nucleotides in double-stranded PCR-amplified DNA. In a typical assay, sequence variation creates a mixed population of heteroduplexes and homoduplexes during reannealing of wild-type and mutant DNA. When this mixed population is analyzed by DHPLC under partially denaturing temperatures, the heteroduplex molecules elute from the column prior to the homoduplex molecules, because of their reduced melting temperatures (Kota et al., Genome 44: 523-528 (2001)). An example of an instrument used to analyze SNPs by DHPLC is the WAVE® HS System from Transgenomic, Inc. (Omaha, Nebr.).

A microarray-based method for high-throughput monitoring of plant gene expression can be utilized as a genetic marker system. This ‘chip’-based approach involves using microarrays of nucleic acid molecules as gene-specific hybridization targets to quantitatively or qualitatively measure expression of plant genes (Schena et al., Science 270:467-470 (1995), the entirety of which is herein incorporated by reference; Shalon, Ph.D. Thesis. Stanford University (1996), the entirety of which is herein incorporated by reference). Every nucleotide in a large sequence can be queried at the same time. Hybridization can be used to efficiently analyze nucleotide sequences. Such microarrays can be probed with any combination of nucleic acid molecules. Particularly preferred combinations of nucleic acid molecules to be used as probes include a population of mRNA molecules from a known tissue type or a known developmental stage or a plant subject to a known stress (environmental or man-made) or any combination thereof (e.g. mRNA made from water stressed leaves at the 2 leaf stage). Expression profiles generated by this method can be utilized as markers.

Polymorphisms can also be identified by Single Strand Conformation Polymorphism (SSCP) analysis. SSCP is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5: 874-879 (1989)). Under denaturing conditions, a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis.

In one embodiment of the method described herein, the oligonucleotide probe is adjacent to a polymorphic nucleotide position in the high-protein QTL. For the purpose of QTL mapping, the markers included must be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes. In one embodiment of the method described herein, genotyping comprises detecting a haplotype.

GEMMA GWAS methods can be used to identify the top genomic regions (QTL) associated with high protein trait.

In one embodiment, the method further comprises determining the protein content of the second population of pea plants or seeds, wherein the second population of pea plants or seeds have an increased level of protein when compared to a population of pea plants or seeds lacking one or more high-protein QTLs selected from the group consisting of Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps02_432513197, Ps02_440456554, Ps03_158264810, Ps03_205819517, Ps03_206829164, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps06_410567663, Ps06_427519500, Ps06_446483044, Ps07_9801763, Ps07_20773355, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897. Determining protein content in a seed or plant is well known to the person of skill in the art and any such methods known to a skilled artisan may be used.

The genetic linkage of additional marker molecules can be established by a gene mapping model such as, without limitation, the flanking marker model reported by Lander and Botstein, Genetics, 121:185-199 (1989), and the interval mapping, based on maximum likelihood methods described by Lander and Botstein, Genetics, 121:185-199 (1989), and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y., the manual of which is herein incorporated by reference in its entirety). Use of Qgene software is a particularly preferred approach.

A maximum likelihood estimate (MLE) for the presence of a marker is calculated, together with an MLE assuming no QTL effect, to avoid false positives. A log10 of an odds ratio (LOD) is then calculated as: LOD=log10 (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of markers and the length of the genome. Graphs indicating LOD thresholds are set forth in Lander and Botstein, Genetics, 121:185-199 (1989), and further described by Arús and Moreno-Gonzalez, Plant Breeding, Hayward, Bosemark, Romagosa (eds.) Chapman & Hall, London, pp. 314-331 (1993).

Additional models can be used. Many modifications and alternative approaches to interval mapping have been reported, including the use of non-parametric methods (Kruglyak and Lander, Genetics, 139:1421-1428 (1995), the entirety of which is herein incorporated by reference). Multiple regression methods or models can also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen and Stam, Genetics, 136:1447-1455 (1994) and Zeng, Genetics, 136:1457-1468 (1994). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng, Genetics, 136:1457-1468 (1994)). These models can be extended to multi-environment experiments to analyze genotype-environment interactions (Jansen et al., Theo. Appl. Genet. 91:33-37 (1995).

Selection of appropriate mapping populations is important to map construction. The choice of an appropriate mapping population depends on the type of marker systems employed (Tanksley et al., Molecular mapping of plant chromosomes. chromosome structure and function: Impact of new concepts J. P. Gustafson and R. Appels (eds.). Plenum Press, New York, pp. 157-173 (1988), the entirety of which is herein incorporated by reference). Consideration must be given to the source of parents (adapted vs. exotic) used in the mapping population. Chromosome pairing and recombination rates can be severely disturbed (suppressed) in wide crosses (adaptedxexotic) and generally yield greatly reduced linkage distances. Wide crosses will usually provide segregating populations with a relatively large array of polymorphisms when compared to progeny in a narrow cross (adaptedxadapted).

An F2 population is the first generation of selfing after the hybrid seed is produced. Usually a single F1 plant is selfed to generate a population segregating for all the genes in Mendelian (1:2:1) fashion. Maximum genetic information is obtained from a completely classified F2 population using a codominant marker system (Mather, Measurement of Linkage in Heredity: Methuen and Co., (1938), the entirety of which is herein incorporated by reference). In the case of dominant markers, progeny tests (e.g., F3, BCF2) are required to identify the heterozygotes, thus making it equivalent to a completely classified F2 population. However, this procedure is often prohibitive because of the cost and time involved in progeny testing. Progeny testing of F2 individuals is often used in map construction where phenotypes do not consistently reflect genotype (e.g. disease resistance) or where trait expression is controlled by a QTL. Segregation data from progeny test populations (e.g. F3 or BCF2) can be used in map construction. Marker-assisted selection can then be applied to cross progeny based on marker-trait map associations (F2, F3), where linkage groups have not been completely disassociated by recombination events (i.e., maximum disequilibrium).

In certain embodiments of the method described herein, genotyping comprises assaying for a deletion marker. As with SNP markers, deletion markers can be identified or detected using standard nucleotide amplification techniques and/or oligonucleotide probes. In specific embodiments, deletion makers can be detected by amplifying a region comprising the complete deletion using primers located upstream (5′) and downstream (3′) of the anticipated deletion. Oligonucleotide probes can be designed to specifically detect a deletion marker by detecting the junction of the ligation of the upstream (5′) and downstream (3′) regions of the anticipated deletion. Oligo nucleotide probes disclosed herein can be labelled with any detection label used in the art including, but not limited to, fluorescent probes and radiolabeled probes.

### 2.4. Breeding of High-Protein Pea Plants

High-protein pea plants of the present disclosure can be part of or generated from a breeding program. The choice of breeding method depends on the mode of plant reproduction, the heritability of the trait(s) being improved, and the type of cultivar used commercially (e.g., F1 hybrid cultivar, pureline cultivar, etc.). A cultivar is a race or variety of a plant that has been created or selected intentionally and maintained through cultivation.

Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Peas: Improvement, Production and Uses, 2nd Edition, Monograph, 16:249 (1987); Fehr, Principles of Variety Development, Theory and Technique, (Vol. 1) and Crop Species Pea (Vol. 2), Iowa State Univ., Macmillan Pub. Co., NY, 360-376 (1987).

Selected, non-limiting approaches for breeding the plants of the present invention are set forth below. A breeding program can be enhanced using marker assisted selection (MAS) of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination event, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new pea cultivars requires the development and selection of pea varieties, the crossing of these varieties and selection of superior hybrid crosses. The hybrid seed can be produced by manual crosses between selected male-fertile parents or by using male sterility systems. Hybrids are selected for certain single gene traits such as pod color, flower color, seed yield, pubescence color or herbicide resistance which indicate that the seed is truly a hybrid. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Pedigree breeding is used commonly for the improvement of self-pollinating crops. Two parents who possess favorable, complementary traits (e.g., high protein) are crossed to produce an F1. An F2 population is produced by selfing one or several F1's. Selection of the best individuals in the best families is selected. Replicated testing of families can begin in the F4 generation to improve the effectiveness of selection for traits with low heritability. At an advanced stage of inbreeding (i.e., F6 and F7), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

In a multiple-seed procedure, pea breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh pods with a machine than to remove one seed from each by hand for the single-seed procedure. The multiple-seed procedure also makes it possible to plant the same number of seed of a population each generation of inbreeding.

Descriptions of other breeding methods that are commonly used for different traits and crops can be found in one of several reference books (e.g., Fehr, Principles of Cultivar Development Vol. 1, pp. 2-3 (1987)).

### 2.5 Plants, Plant Parts, Plant Cells, and Plant Products

Disclosed herein are high-protein pea plants, plant parts (e.g., juice, pulp, seed, grain, fruit, flowers, nectar, embryos, pollen, ovules, leaves, stems, branches, bark, kernels, ears, cobs, husks, stalks, roots, root tips, anthers, etc.), or plant products produced by the methods provided herein. Progeny, variants, and mutants of the produced plants are also included within the scope of the invention, provided that they comprise the high-protein phenotype.

“Plant products,” as used herein, refers to any product or composition produced from the plant, including any oil products, sugar products, fiber products, protein products (such as protein concentrate, protein isolate, flake, or other protein product), seed hulls, meal, or flour, for a food, feed, aqua, or industrial product, plant extract (e.g., sweetener, antioxidants, alkaloids, etc.), plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), grains, plant protein composition, plant oil composition, and food and beverage products containing plant compositions (e.g., plant parts, plant extract, plant concentrate, plant powder, plant protein, plant oil, and plant biomass) described herein. Plant parts and plant products provided herein can be intended for human or animal consumption.

As used herein, a “protein product” or “protein composition” refers to any protein composition or product isolated, extracted, and/or produced from plants or plant parts (e.g., seed) and includes isolates, concentrates, and flours, e.g., pea protein composition, pea protein concentrate (SPC), pea protein isolate (SPI), pea flour, flake, white flake, texturized vegetable protein (TVP), or textured pea protein (TPP)). A protein composition can be a concentrated protein solution (e.g., yellow pea protein concentrate solution) in which the protein is in a higher concentration than the protein in the plant from which the protein composition is derived.

“White flake protein” as used herein refers to a protein composition obtained by de-hulling, flaking, and defattening plants or plant parts (e.g., legume plants or plant parts) by solvent (e.g., hexane) extraction, with limited use of heat to run off the solvent (Lusas and Riaz, 1995). White flake protein is an intermediate product in the production of plant protein concentrates and isolates.

In contrast to conventional toasted plant meal (e.g., pea protein meal), white flakes contains undenaturated proteins due to the very mild heat treatment. Thus, little or no reduction of protease inhibitors would be expected. The undenaturated proteins in white flakes may be advantageous in supporting binding properties during production of the extruded compound feed. White flakes can be used for human and animal consumption, including as a source of protein in aquaculture feeds for any type of fish or aquatic animal in a farmed or wild environment.

The protein composition can comprise multiple proteins as a result of the extraction or isolation process. In specific embodiments, the protein composition can further comprise stabilizers, excipients, drying agents, desiccating agents, anti-caking agents, or any other ingredient to make the protein fit for the intended purpose. The protein composition can be a solid, liquid, gel, or aerosol and can be formulated as a powder. The protein composition can be extracted in a powder form from a plant and can be processed and produced in different ways, such as: (i) as an isolate—through the process of wet fractionation, which has the highest protein concentration; (ii) as a concentrate—through the process of dry fractionation, which are lower in protein concentration; and/or (iii) in textured form—when it is used in food products as a substitute for other products, such as meat substitution (e.g. a “meat” patty). Protein isolate can be derived from defatted pea flour with a high solubility in water, as measured by the nitrogen solubility index (NSI). The aqueous extraction is carried out at a pH below 9. The extract is clarified to remove the insoluble material and the supernatant liquid is acidified to a pH range of 4-5. The precipitated protein-curd is collected and separated from the whey by centrifuge. The curd can be neutralized with alkali to form the sodium proteinate salt before drying. Protein concentrate can be produced by immobilizing the pea globulin proteins while allowing the soluble carbohydrates, whey proteins, and salts to be leached from the defatted flakes or flour. The protein is retained by one or more of several treatments: leaching with 20-80% aqueous alcohol/solvent, leaching with aqueous acids in the isoelectric zone of minimum protein solubility, pH 4-5; leaching with chilled water (which may involve calcium or magnesium cations), and leaching with hot water of heat-treated defatted protein meal/flour (e.g., pea meal/flour). Any of the process provided herein can result in a product that is 70% protein, 20% carbohydrates (2.7 to 5% crude fiber), 6% ash and about 1% oil, but the solubility may differ. As an example, one ton (t) of defatted pea flakes can yield about 750 kg of pea protein concentrate. “Texturized vegetable protein” (TVP), “Textured vegetable protein,” “textured pea protein” (TPP), pea meat, or pea chunks refers to a defatted plant (e.g., pea) flour product, a by-product of extracting plant (e.g., pea) oil. It can be used as a meat analogue or meat extender. It is quick to cook, with a protein content comparable to certain meats. TVP can be produced from any protein-rich seed meal left over from vegetable oil production. A wide range of pulse seeds other than pea, such as lentils, peas, and fava beans, or peanut may be used for TVP production. TVP can be made from high protein (e.g., 50%) pea isolate, flour, or concentrate, and can also be made from cottonseed, wheat, and oats. It is extruded into various shapes (chunks, flakes, nuggets, grains, and strips) and sizes, exiting the nozzle while still hot and expanding as it does so. The defatted thermoplastic proteins are heated to 150-200° C., which denatures them into a fibrous, insoluble, porous network that can soak up as much as three times its weight in liquids. As the pressurized molten protein mixture exits the extruder, the sudden drop in pressure causes rapid expansion into a puffy solid that is then dried. As much as 50% protein when dry, TVP can be rehydrated at a 2:1 ratio, which drops the percentage of protein to an approximation of ground meat at 16%. TVP can be used as a meat substitute. When cooked together, TVP can help retain more nutrients from the meat by absorbing juices normally lost. Also provided herein are methods of isolating, extracting, or preparing any of the protein compositions or protein products provided herein from plants or plant parts.

Also provided herein are food and/or beverage products containing plant compositions (e.g., plant parts, plant extract, plant concentrate, plant powder, plant protein, and plant biomass) described hereinabove, such as plant compositions derived from the plants or plant parts of the present disclosure. Such food and/or beverage products include, without limitation, shakes, juices, health drinks, alternative meat products (e.g., meatless burger patties, meatless sausages, etc.), alternative egg products (e.g., eggless mayo), and non-dairy products (e.g., non-dairy whipped toppings, non-dairy milk, non-dairy creamer, non-dairy milk shakes, etc. and condiments. A food and/or beverage product that contains plant compositions obtained from plants or plant parts of the present disclosure can have desired traits, compared to a similar or comparable food and/or beverage product that contains plant compositions obtained from a control plant or plant part.

Plant parts (e.g., seeds) and plant products (e.g., plant biomass, seed compositions, protein compositions, food and/or beverage products) produced by the methods provided herein can be meant for consumption by agricultural animals or for use as feed in an agriculture or aquaculture system. In specific embodiments, plant parts and plant products produced according to the methods provided herein include animal feed (e.g., roughages-forage, hay, silage; concentrates-cereal grains, pea cake) intended for consumption by bovine, porcine, poultry, lambs, goats, or any other agricultural animal. In some embodiments, plant parts and plant products produced according to the methods include aquaculture feed for any type of fish or aquatic animal in a farmed or wild environment including, without limitation, trout, carp, catfish, salmon, tilapia, crab, lobster, shrimp, oysters, clams, mussels, and scallops.

Plants, plant parts, or plant products produced by the method of producing a population of high-protein pea plants or seeds provided herein can have a greater frequency of the high-protein molecular marker and/or higher protein content than the starting, or control population of pea plants, plant parts, or plant products. Plants, plant parts, or plant products produced by the method of introgressing a high-protein QTL can have a greater frequency of the high-protein QTL and/or higher protein content than the starting, or control population of pea plants, plant parts, or plant products.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting. Unless otherwise noted, all parts and percentages are by dry weight.

### EXAMPLES

### Example 1. Identifying SNP Markers Associated With High-Protein Phenotype in Pea Seeds

A genotyping-by-sequence (GBS) panel with genome-wide association study (GWAS)-based and stride-selected markers for protein prediction was designed. These markers have high effect on protein in a population of yellow pea breeding lines in a LASSO genomic prediction model and/or GWAS. High protein markers in the GBS panel include 3 GWAS identified markers using linkage disequilibrium (LD) pruned genome-wide efficient mixed model analysis (GEMMA) on chromosome 3 or 3LG5 (Ps03_531014546, Ps03_531232613, Ps03_531239107) and 144 LASSO identified markers on chromosomes 1-7 or scaffolds of the Cameor v1a reference genome. The identified high protein markers are presented in Table 1. Each marker identified in Table 1 includes the SNP marker with 100 nucleotide representative genomic sequences upstream and downstream of the SNP marker. Each marker (such as “Ps01_20222535” comprises the SNP marker indicated in Table 1, and a nucleic acid sequence that has at least 90% sequence identity with the nucleic acid sequence indicated in Table 1. “Protein_LASSO_Coeff” refers to protein LASSO coefficient, which represents strength of association between the marker and increased protein content, with a higher absolute value indicating higher association with increased protein content. A positive protein LASSO coefficient indicates that the alternate allele of the marker is associated with high protein content. A negative protein LASSO coefficient indicates that the reference allele of the marker is associated with high protein content.

Details on the method for GEMMA can be found in Xiang Zhou and Matthew Stephens 2012 Nature Genetics 44, 821-824, herein incorporated by reference in its entirety.

The sequence upstream and downstream of the SNP may vary. Each marker listed in Table 1 can have a sequence that has at least 90% identity with the respective sequence in Table 1. Each marker listed in Table 1 (referred to as “marker AA”) can also refer to an SNP marker at position 101 of the nucleic acid sequence identified for the marker AA (referred to as “SEQ ID NO: BB”) in a genomic region comprising the nucleic acid sequence of SEQ ID NO: BB, or at a corresponding position of a genomic region at least about 50, 100, 150, or 200 nucleotides of which is aligned to SEQ ID NO: BB for maximum homology (e.g., 90%, 91%, 92%, 93%, 94%, 95%, 96%, 97%, 98%, or 99% homology) when using standard alignment parameters. For example, the SNP marker identified as Ps05_49389403 or chr5-1 can have a sequence that has at least 90% identity with SEQ ID NO: 148. The marker identified as Ps05_49389403 or chr5-1 can also refer to a SNP marker at position 101 of a nucleic acid sequence of SEQ ID NO: 148 in a genomic region comprising the nucleic acid sequence of SEQ ID NO: 148, or at a corresponding position of a genomic region at least about 50, 100, 150, or 200 nucleotides of which is aligned to SEQ ID NO: 148 for maximum homology (e.g., 90%, 91%, 92%, 93%, 94%, 95%, 96%, 97%, 98%, or 99% homology) when using standard alignment parameters. The SNP marker identified as Ps03_531239107 or chr3-4 can have a sequence that has at least 90% identity with SEQ ID NO: 47. The marker identified as Ps03_531239107 or chr3-4 can also refer to an SNP marker at position 101 of a nucleic acid sequence of SEQ ID NO: 47 in a genomic region comprising the nucleic acid sequence of SEQ ID NO: 47, or at a corresponding position of a genomic region at least about 50, 100, 150, or 200 nucleotides of which is aligned to SEQ ID NO: 47 for maximum homology (e.g., 90%, 91%, 92%, 93%, 94%, 95%, 96%, 97%, 98%, or 99% homology) when using standard alignment parameters.

TABLE 1
SNP Markers Associated with High-Protein Pea Phenotype

| | SNP | | | | Cameor | | |
|---|---|---|---|---|---|---|---|
| | alleles | | Sequence (“[[X/Y]]” indicates | Cameor | v1a | Genetic | Protein_ |
| | (reference/ | SEQ ID | the reference/alternate | v1a | Physical | Position | LASSO_ |
| Marker | alternate) | NO: | alleles of SNP) | chromosome | Position | (cM) | Coeff |
| Chr5-1 | A/G | 148 | CGTRAGTAGGATTAGGCAATRC | 5 | 36835261 | 9.6 | NA |
| Ps05_49389403 | | | AACATAACRTTAATTCACTAGCC | | | | |
| | | | AYTACTCGGGGTCTCCTATCYCT | | | | |
| | | | ATTCAACCAACATAAGCACAAC | | | | |
| | | | AATTRCCCWA[[G/A]]GTCTAACAC | | | | |
| | | | ATAGACTAATGGTCAGTATTYCC | | | | |
| | | | TATGTGCCCAAAATTAGATKAA | | | | |
| | | | AAGAGTATCTCACATAAAAARC | | | | |
| | | | ATTAYGAACAAGAAGAAATTGA | | | | |
| | | | AT | | | | |
| Ps01_20222535 | G/A | 1 | ATAACCAATTAAAAATGAGATT | chr1LG6 | 20877277 | 8.183 | 0.034 |
| | | | TYATTTGCTGCAAATGTTATGAC | | | | |
| | | | TATAAACCACTTAAAATAAGAA | | | | |
| | | | ATGATGGTGTATGCTCTACTTCT | | | | |
| | | | TAAAGAACTT[[G/A]]TTGATTTGG | | | | |
| | | | TTTGACTTGTCTTGATGCARTAA | | | | |
| | | | CCAAGATAAGTTGCAATTAATG | | | | |
| | | | GAAAAAAAAYAATGAACATACT | | | | |
| | | | TCATAARTAGTGYTTGTAAAYCA | | | | |
| | | | T | | | | |
| Ps01_22514126 | T/C | 2 | CCCTCCTCGGGAAGATCAGCATC | chr1LG6 | 23108565 | 9.536 | −0.025 |
| | | | GCAAAAGCTCAAATTGTTACAA | | | | |
| | | | GCAGTTATGTTTGCAACAATGCT | | | | |
| | | | ATCAAACTGTTCTATTGTGACAT | | | | |
| | | | CGTGATCCA[[T/C]]GTATGACACG | | | | |
| | | | TCCAACACCTTTTGCAAAGCTTC | | | | |
| | | | ATGGTGTGGCTCGGAATTCATCA | | | | |
| | | | GCAAAGATAGGACARATATCTT | | | | |
| | | | TGACGGAGTTTGAAGTAGCTGA | | | | |
| Ps01_55991509 | C/T | 3 | TCACTGAGGATACAAAGAAGAT | chr1LG6 | 51330566 | 20.005 | 0.089 |
| | | | GTATTCARGAGYAATGAAAGAA | | | | |
| | | | GATGATCAAAYAGAGAACTCAA | | | | |
| | | | CTCTGATGGGGATCATCTMTGG | | | | |
| | | | AATAAGAGTAAA[[C/T]]AAAGGT | | | | |
| | | | AAGACCTTAACCATAGCTGGGG | | | | |
| | | | ATAGGCACAGTCAAAGCTTCCA | | | | |
| | | | ACTGYAGGAGATAACACTCTGC | | | | |
| | | | ATAGGAGAMGTCTTCCAGGAGC | | | | |
| | | | AAACAA | | | | |
| Ps01_78756169 | G/C | 4 | CTTGACTYTGAAAAGTCAAAGC | chr1LG6 | 72083191 | 26.460 | −0.053 |
| | | | TAGTTGAGAAATTTGTAGATATG | | | | |
| | | | GAGATCACTTACTCAGGTTCTGA | | | | |
| | | | AGGGAAGAATTTAGAAGCCATA | | | | |
| | | | AAAGCTGAAG[[G/C]]TAAAGACTC | | | | |
| | | | TGAAGATCCTTAGTTAGAAGTTG | | | | |
| | | | TTGAAGCTCAAACTCCTTTGCAC | | | | |
| | | | CAGTTGCCAAGTTATAGTYTATT | | | | |
| | | | CGTYTCTTAATCTCTTTTGCAA | | | | |
| Ps01_87632539 | C/T | 5 | GACTCTAASATAGAAGACTCATT | chr1LG6 | 79225752 | 30.400 | −0.036 |
| | | | CCAATTTTCTAAAAGGTTTCCCA | | | | |
| | | | GACGCTACACTTATAAACTTTAG | | | | |
| | | | GAAGCAACTGTTTTGAATCGACT | | | | |
| | | | ACATATCA[[C/T]]RTGTGCARTCT | | | | |
| | | | AAGGTATTGATCCTAACCATAAT | | | | |
| | | | TCATTTAATTTATTTTTCTTCATT | | | | |
| | | | GAAGTGATAACCTTATATGTTGT | | | | |
| | | | CACCGTAYTAGAAGTCCAA | | | | |
| Ps01_88206114 | G/C | 6 | GTAAYAGTTTTCGAATGGAAAT | scaffold02116 | 29379 | 30.461 | 0.048 |
| | | | CATCTATGAATGTAACAAAGTAT | | | | |
| | | | ATGTTRCATCYAATTAAATCCAC | | | | |
| | | | CTGGATAGGACCRCATACATYA | | | | |
| | | | GAGTATGTGA[[G/C]]TTCAAGAAT | | | | |
| | | | TGCCTTCGACCTGCTTCYTGCAT | | | | |
| | | | CCTTACTGAAGTTATTCTTGTGC | | | | |
| | | | TACTTCGGCTGTACACATTCTTC | | | | |
| | | | ACACACTTCATTTGGAATGYCR | | | | |
| Ps01_95579585 | A/G | 7 | CAGACAAAGAGGAMTATCCAGT | chr1LG6 | 87185358 | 33.400 | 0.029 |
| | | | CTTTGGAGGAGTTGTTGAGGGCT | | | | |
| | | | TGTGTTCTAGAGCATGACRRTAC | | | | |
| | | | TTGGATAGTTACTTKCCTCTGAT | | | | |
| | | | AGAGTTCAY[[A/G]]TACAATAACA | | | | |
| | | | AYTACCMTTCTAGTATTGGGAT | | | | |
| | | | GACACTATTCGAGGTGTTGTATG | | | | |
| | | | ATAGGAGATGTAGGACTCCTCT | | | | |
| | | | ATGYTGGTATGATTCTGGAGAG | | | | |
| | | | A | | | | |
| Ps01_113369982 | T/C | 8 | ATTTGGMTCCARATTCGAGGTGT | scaffold04655 | 36097 | 37.744 | 0.034 |
| | | | TCAGTGATCAAAAGAGTTTGAA | | | | |
| | | | GTATTTATTCAATCAAAAAGAKT | | | | |
| | | | TSACWATGAGGCAGAAGAGATG | | | | |
| | | | GCTYGAATTC[[T/C]]TRAAGGATT | | | | |
| | | | ATTATTTTAGATTGAGCTACCCT | | | | |
| | | | CCTGGTAAARCCAATKTTGYAA | | | | |
| | | | CTRATGCGCTAWGTAGGAAGTT | | | | |
| | | | GTTACATATGTCGATGCTGATGA | | | | |
| | | | T | | | | |
| Ps01_113369984 | G/A | 9 | TTGGMTCCARATTCGAGGTGTTC | scaffold04655 | 36095 | 37.744 | −0.046 |
| | | | AGTGATCAAAAGAGTTTGAAGT | | | | |
| | | | ATTTATTCAATCAAAAAGAKTTS | | | | |
| | | | ACWATGAGGCAGAAGAGATGG | | | | |
| | | | CTYGAATTCYT[[G/A]]AAGGATTA | | | | |
| | | | TTATTTTAGATTGAGCTACCCTC | | | | |
| | | | CTGGTAAARCCAATKTTGYAACT | | | | |
| | | | RATGCGCTAWGTAGGAAGTIGT | | | | |
| | | | TACATATGTCGATGCTGATGATT | | | | |
| | | | T | | | | |
| Ps01_120406755 | T/C | 10 | AGGTATTCTCCCTTTCCAGATTT | chr1LG6 | 108299170 | 39.075 | −0.040 |
| | | | CATATAAAGTAGTAGGAGTCCC | | | | |
| | | | TTTCTTCAAGGTTACTCTTGAAC | | | | |
| | | | ATGTTCATAGCTTCAGCCCAAAA | | | | |
| | | | GTGGTAGGG[[T/C]]AATTTCTTAG | | | | |
| | | | CATGAATCATAGCTCTGGCTGAG | | | | |
| | | | TCTTGGAGAGTTCTATTTTTCCTT | | | | |
| | | | TCTATCACACCATTTTGCTGAGG | | | | |
| | | | AGTAATGGGAGATGAGAACC | | | | |
| Ps01_160458316 | G/A | 11 | AAYGAGATTTGTTTAKGRCCTTA | scaffold00066 | 157869 | 46.833 | −0.031 |
| | | | AAARMTTTYGATGATAACAAAA | | | | |
| | | | TATTTAAARAACAAAAGAGTTT | | | | |
| | | | GCTAACATTTTGTTTAAGTGTGC | | | | |
| | | | AAGATCACAA[[G/A]]CATTGAATT | | | | |
| | | | AATTCTGCTTGTTAGAATTTGAT | | | | |
| | | | TGCCTCYGAAGGCATCAGATTCT | | | | |
| | | | GGCATCATGACGCAACTTCTGAT | | | | |
| | | | GAYAACTCAAAYTCTGAAGGTG | | | | |
| Ps01_264925535 | C/T | 12 | GAGCTTCAAGGTCCTAAAAAAA | chr1LG6 | 194893633 | 50.698 | 0.026 |
| | | | TGGTGAATGGAGGTTTCCCTTTT | | | | |
| | | | CAAATGTCGATGCTCACAAAGA | | | | |
| | | | ACAACTATGGCAATTGGAGTAT | | | | |
| | | | CAAGATGAAGG[[C/T]]GCTACTAG | | | | |
| | | | AAGCTCAAGATGTGTGAGATAT | | | | |
| | | | CGTTAAGAAAGACTTCAAGGAG | | | | |
| | | | CAAAATGAAGTCTCGCTAAGCC | | | | |
| | | | AAGGTGTAAAGGAGACATTGAA | | | | |
| | | | GGAG | | | | |
| Ps01_279286967 | A/G | 13 | CAACATTGTATTTATATTGCTTG | chr1LG6 | 122338117 | 52.017 | −0.034 |
| | | | AYRAAAGCTTCGCCAAGATCAT | | | | |
| | | | TGAAGGAGMGGATGTTTGCACT | | | | |
| | | | ATCCAAACCCATATACCATCTRA | | | | |
| | | | GAGCGGCACC[[A/G]]GACAGGCT | | | | |
| | | | GTCCTGGAAATAGTGGATAAGG | | | | |
| | | | AGTTGATCGTTGTCTRTCTGAGT | | | | |
| | | | CAMMATCTTRCGTGCGTACATC | | | | |
| | | | ACAAGATGGCTGRGAGGGCAAG | | | | |
| | | | TGT | | | | |
| Ps01_280789385 | A/G | 14 | TTTCTCACTTGTGTATGGAATGG | scaffold03789 | 72152 | 52.644 | 0.092 |
| | | | AAGTGGTGYTGCCGGTTGAGGT | | | | |
| | | | CCAGCTTCCATCGTTGAGAGTCC | | | | |
| | | | TGCTGGATGTGAAATTGAAAGA | | | | |
| | | | GGCTGAATGG[[A/G]]TAAGAACTC | | | | |
| | | | GGTATGAGGAGTTGAGCCTGAT | | | | |
| | | | TGAGGAAAAGAGGCTGGCGTCC | | | | |
| | | | ATTTGCCATGGGCAGTTGTACCA | | | | |
| | | | ACAGCGAATGAAGCATGCTTTT | | | | |
| | | | GA | | | | |
| Ps01_300614888 | G/A | 15 | CCATGCAAGCATGCAAGCATGC | chr1LG6 | 30542636 | 56.081 | 0.040 |
| | | | AAGAGTGATTTTCRWGTTTTGCC | | | | |
| | | | AAAAATGGAAGTGTGCAAATAT | | | | |
| | | | CAGTCCCATTGGCCTATAWATA | | | | |
| | | | TAAGCTCTCTT[[G/A]]CTCAKAAA | | | | |
| | | | TAGGAACCTCATGTGCAAGCTTT | | | | |
| | | | GATTCARCAACCCTAAACCCTCA | | | | |
| | | | CCATTAAAGGATAAGCTTGAAG | | | | |
| | | | ATTTTCTTTGAAAATCGAGTTTC | | | | |
| | | | A | | | | |
| Ps01_324252121 | C/T | 16 | CAAAAGGATTAAAAAMACAAAT | chr1LG6 | 116776833 | 60.275 | 0.035 |
| | | | AAAAATCAGGCATAAAAGTGCT | | | | |
| | | | CAAAAAATTAAAATGGATACAC | | | | |
| | | | TACAATAATCTGCGCGAAATAA | | | | |
| | | | ACTCGAAACATA[[C/T]]AGGTTTT | | | | |
| | | | TGTCAAATTTTGAAATGGAAAA | | | | |
| | | | CGCYAGGTTAAAATGCTCAGAC | | | | |
| | | | GGATTGAAATGTGCCAAAAAAT | | | | |
| | | | TTAGCCGAAAAATAAATCGAGC | | | | |
| | | | ACACY | | | | |
| Ps01_349096914 | G/A | 17 | TCATTCTCTCTCTCTCTCTCTCTC | chr1LG6 | 288453266 | 65.200 | 0.038 |
| | | | TCTCTCTCTCTCTCTTTCTCTCTC | | | | |
| | | | TCTCTCTCTCCTTACTAAAAGTG | | | | |
| | | | TCTCACACACTAACAAATCCTAC | | | | |
| | | | TGTGGC[[G/A]]AAGACTAAAATGG | | | | |
| | | | ATGTGGTGAGATTTCCTATCYRT | | | | |
| | | | ACTAGTTGTCATGTTAYCATTAA | | | | |
| | | | TGAAGTTCTTAAAGTTAAAATCA | | | | |
| | | | ATGGGATCTYCTTCAGAA | | | | |
| Ps01_367706416 | G/A | 18 | GAAATTTAAATTTTATATTGCAA | chr1LG6 | 306857129 | 70.156 | −0.070 |
| | | | CTTCACATAATTTATTGTATATT | | | | |
| | | | ACTATTATTATCGATTTGCTTAT | | | | |
| | | | CAACAACATTTGATATTTTTTGC | | | | |
| | | | TATGATTT[[G/A]]CTTGCTTAATAT | | | | |
| | | | TCAACCAAATTTTGATTAATTCA | | | | |
| | | | CGTTAGGTTAAAAACTCTTGGCT | | | | |
| | | | GTAATATTTTTCAATTAAACTAA | | | | |
| | | | CTAACTCAATCTATTGAAT | | | | |
| Ps01_380906255 | G/A | 19 | TTCACTATTACTTCTTCTTACATA | chr1LG6 | 45686305 | 75.235 | −0.047 |
| | | | TACTCCATGYTCAKTTGTGCACT | | | | |
| | | | TATAAATTCCTTCTCTSTTAGGA | | | | |
| | | | AATTGTCTATCTTCTTATTTCAA | | | | |
| | | | GCTCWTG[[G/A]]AGCYTGCTTAAG | | | | |
| | | | TCCATAYAGGGCTTTATGCAGCC | | | | |
| | | | TGCATACCTTGCTTTCTTMGCCA | | | | |
| | | | TGTTTMRTAAATCCAACTAGTTG | | | | |
| | | | TGCAACATAAAMTTCKTCA | | | | |
| Ps01_436651445 | A/G | 20 | TACTGATGAATATTTTTATATGA | chr1LG6 | 367968951 | 99.418 | 0.052 |
| | | | TTGTGAGATTTTATTGTGATATG | | | | |
| | | | GATGAGATGTGAATACATGATA | | | | |
| | | | YTCAAGATGCAGTGTGTGAATCT | | | | |
| | | | ATCATGATT[[A/G]]TTTATGCACC | | | | |
| | | | ATTTATTATTTGAATGTATTCTC | | | | |
| | | | ACCACTTTTTGTATTGTTGCGTG | | | | |
| | | | TGCGCTCCTATGATAACAAATAA | | | | |
| | | | TGAGATATTTGAGTATGAACT | | | | |
| Ps01_440892085 | A/G | 21 | TAAAATCATATACCATCCAAGA | chr1LG6 | 371072249 | 101.091 | −0.044 |
| | | | GYTCCTTCTTTAAAGGTACCAAC | | | | |
| | | | CATGAGTTTACATTTAAGGGAGT | | | | |
| | | | CAGAAGCCCCTACTATTTCCATC | | | | |
| | | | TGATTACTG[[A/G]]TCRTAATAAT | | | | |
| | | | ATGCTCMTGTRGGTTGGTTTTAT | | | | |
| | | | CATTAAATGACACCAGTGAAGG | | | | |
| | | | CAGTTTAAAGTTTTCTRGGACTT | | | | |
| | | | GATCATCCCAGATAGCCTCRGA | | | | |
| Ps02_17543117 | T/C | 22 | CATCTAAGCTCCCCATGAGCTAT | chr2LG1 | 10842575 | 9.100 | −0.068 |
| | | | TTTCTRAGCCSAGATGTCSAGAA | | | | |
| | | | GTTTTGGAGCGATATTACRTGTG | | | | |
| | | | GAAGTAGTCCTGACCCTTCCACC | | | | |
| | | | CAAGTCTC[[T/C]]AGMAAGATTCA | | | | |
| | | | CAACCCTGCTTTCAGGTACTTCC | | | | |
| | | | AGATGATCATTTCCCACACCTTC | | | | |
| | | | CTGAGGAAGAGTGACCCTGATA | | | | |
| | | | TGCATGTGAGTGTTGAAGAGA | | | | |
| Ps02_64161520 | C/T | 23 | CGRCCRTTATTCTTATTTCTAAA | chr2LG1 | 26206979 | 22.300 | −0.027 |
| | | | GGTTTTCTCGATATTTTCCTATTC | | | | |
| | | | CTTTATTAGGAATAAATAAAGTT | | | | |
| | | | CGGTGGCGACTCTGTTTCGAACA | | | | |
| | | | ATTTTTC[[C/T]]GCGTTCCATCGCG | | | | |
| | | | AGGGATCGCATTATCATCATTTT | | | | |
| | | | TTTCGAGGTGCGACAGAACTCA | | | | |
| | | | CTACACAGCTCTTTTATCATCTT | | | | |
| | | | GTTGTTCAAGTTAGATCCA | | | | |
| Ps02_149117835 | T/C | 24 | GTTGTTTTCCAAAATATGACGGT | scaffold02021 | 8290 | 39.609 | −0.045 |
| | | | AAGACGGCTTGCCCCAGTATTTT | | | | |
| | | | GAATCTTATAGAGGTATGCCCCT | | | | |
| | | | GATTGGCTAACTTTTGGAGTGAC | | | | |
| | | | GGGCTTCA[[T/C]]AGTACCATGGT | | | | |
| | | | AGTGACTTGCYCCAGTATGAGC | | | | |
| | | | CTTGGAGAGACCTGTTGCGACTC | | | | |
| | | | AAAGTGATTGCCCCAAATTCACT | | | | |
| | | | GAATTTTGAAACAACTTACAC | | | | |
| Ps02_162193050 | A/G | 25 | TCAAAGATCTCAAYCCAATTTAT | chr2LG1 | 119392808 | 41.660 | −0.041 |
| | | | AATTCAGCAACCTAAATTGGAC | | | | |
| | | | GTTATCAACATTGATTCTAGATG | | | | |
| | | | RCACCCAAAYAAAGAATGATTA | | | | |
| | | | TGTGTTGTTT[[A/G]]TTTATGTGTA | | | | |
| | | | TGCATAGAATGTGGGTTGAACA | | | | |
| | | | TGATGAGAACTCTTTGAAATCTC | | | | |
| | | | GATTTCATGTAGRTTTACTCTAG | | | | |
| | | | AACCTTACTAGTAATGATGCAT | | | | |
| Ps02_249953551 | A/G | 26 | TTTGAAACCCATTTCTTTGGCGA | chr2LG1 | 169314375 | 47.300 | −0.055 |
| | | | ATTCCTGCATTTTCTGACCAGTA | | | | |
| | | | AATACTTATCCTTGATCTGTTGT | | | | |
| | | | GATTGTCTATGGAATTCCAAACC | | | | |
| | | | TATAGATG[[A/G]]TATGTTTTTGG | | | | |
| | | | ATAAATTCGATCACATCCTCTTG | | | | |
| | | | GTTCACATTTGGCAAGGGYATG | | | | |
| | | | GCTTCAATCCACTTTGTAAAATA | | | | |
| | | | ATCGACTCCCACCAATATATA | | | | |
| Ps02_282186543 | G/A | 27 | TTTTAYCCTACCMATTGTGGATA | chr2LG1 | 286755665 | 48.300 | −0.030 |
| | | | ATTTTTRTGGATAYCCACTRGGG | | | | |
| | | | ATGGGTACARTTGCCATCCCTAT | | | | |
| | | | GAGTAGGTGAAAGGTAAGGACA | | | | |
| | | | AGATARCTT[[G/A]]ACGTCCTTGC | | | | |
| | | | TTGCCTGWTGTWTTGGATGMCA | | | | |
| | | | TTTTCGTTATGKATATAACTATG | | | | |
| | | | TTGAGACATTTTGAGAATGATCT | | | | |
| | | | AGTTCCTAGTTTTATTTTGGAT | | | | |
| Ps02_293278647 | G/T | 28 | CTAGAGTGGTTAGGTGACAGAG | scaffold00644 | 91532 | 50.900 | −0.072 |
| | | | GGGGGTTAACTYTTATTTTCATT | | | | |
| | | | TTATTGGTTTTAACATTTTCTTTA | | | | |
| | | | TTTGTGTGTCTSRCGYTGTGGGG | | | | |
| | | | TGTTGATT[[G/T]]TTGTGGTAGYA | | | | |
| | | | TGCACGYGGRATTTATGACARC | | | | |
| | | | ACCCCTRAGTCTTTATGTTGTGG | | | | |
| | | | CCTCRATCTCTTCRATCGTACCT | | | | |
| | | | TGGRCKTGTCATTACCTTACA | | | | |
| Ps02_296256342 | A/G | 29 | TTTTCTGTTTTCCCTTGGAAACA | chr2LG1 | 5787797 | 51.368 | 0.032 |
| | | | ATAAAAGCACGGTGRCGACTCT | | | | |
| | | | GGTTTTATTRACGTTAAGCTTAT | | | | |
| | | | CCATAGGTTGATGGTCATGAATT | | | | |
| | | | TACCACTAT[[A/G]]GAAATTAAGT | | | | |
| | | | GGCGACTCTGYTGGGGAGTAGT | | | | |
| | | | CCTCAGTGGGTTTAGCCTACTTT | | | | |
| | | | TTTATGTGTATATARTTGTATAT | | | | |
| | | | TTGATGTGTGTATACTTKTTTT | | | | |
| Ps02_298578096 | C/T | 30 | TAATGATGAGAGTCAAATAAAA | chr2LG1 | 301660243 | 51.911 | −0.050 |
| | | | AAACAATAAATTAAGWGGAAG | | | | |
| | | | AGAATATCACTAATAAGGATGC | | | | |
| | | | CATCGAGAAAGAGAAAACTTAT | | | | |
| | | | GTACCTYCACCTC[[C/T]]GTATAA | | | | |
| | | | ACCAAAAATATCATACCCTCAA | | | | |
| | | | AGACTAGCCAAAACCAAGAATG | | | | |
| | | | AGGGTCAATTTAARAAATTCATY | | | | |
| | | | TTTCTCCTAAAACAACTTCATTT | | | | |
| | | | TACT | | | | |
| Ps02_313767424 | A/C | 31 | CAACACCAAGTGYCGGATTCAG | chr2LG1 | 87090294 | 54.509 | 0.039 |
| | | | AGAAGAAGAAGGAGTCCCCACA | | | | |
| | | | GGAYGGGCTGCACTATCAACAT | | | | |
| | | | CATCAGCTCTTCTGGCRTCAACC | | | | |
| | | | TGGGGTTTCGR[[A/C]]GGCGGCGA | | | | |
| | | | GAAAACACGACCACTACGGGTC | | | | |
| | | | AAACCACTGYCATCMGAAATAC | | | | |
| | | | TRGTCACAGAAGTGGAGGGTAA | | | | |
| | | | AGGCACCTCCTTCCCATCTTCTA | | | | |
| | | | ATG | | | | |
| Ps02_389051201 | G/A | 32 | AGGRTAATTCAGTATTTTAGTAT | chr2LG1 | 383268619 | 70.521 | 0.035 |
| | | | TTTGTCTAYGTGKCATTATTCTG | | | | |
| | | | TGTATATAAGTAGTAGTGTGCTT | | | | |
| | | | AGCTATTAAGTAACATAACWGT | | | | |
| | | | MTGTACACA[[G/A]]CAGAGCART | | | | |
| | | | GTGAGTGCAACCATTTTGCTGTA | | | | |
| | | | AYTGTTTTTAGCAGAGTAARGG | | | | |
| | | | AAATTTGTTATTCTCTCTTCTCTC | | | | |
| | | | TYTTCTTCCATCTTCATCTTCT | | | | |
| Ps02_432513197 | A/G | 33 | AAACTCATYAAAGTGCTATGAA | chr2LG1 | 420361771 | 89.247 | −0.029 |
| | | | GCAACCTCTRGRYGTCCACCATA | | | | |
| | | | TATTGACCTCAAATCCCATATCT | | | | |
| | | | YWTTCATGYCATCAAAGTCCTT | | | | |
| | | | ACTAGCCGAT[[A/G]]ATGAATGGA | | | | |
| | | | CAACAAYATTGGAACCTTCTAA | | | | |
| | | | ARTATTTAAGCCACATATTTTAG | | | | |
| | | | ACCMTTTAATACCTCATGTTCAA | | | | |
| | | | CTCTAGTGCAAAAKCCTAGAYC | | | | |
| | | | A | | | | |
| Ps02_440456554 | G/C | 34 | CCAATGAAAAAATCAATTCTGCT | chr2LG1 | 426699364 | 92.888 | −0.038 |
| | | | TTCACTTTTTTCCATCTGAAGCA | | | | |
| | | | ATTCATACGTTCTTTTGTGAGTT | | | | |
| | | | TGTAACCTCACATCTTTCACATT | | | | |
| | | | CTCAGTGC[[G/C]]TCAAAATGATT | | | | |
| | | | TGTCAAGAATTTCTCATGYTTCT | | | | |
| | | | TTTGCTAATTCAACATCATTGAC | | | | |
| | | | CTTTTYGAAATTATCTGCATCAA | | | | |
| | | | CATTGATGAATCATAAAGAG | | | | |
| Ps03_158264810 | T/C | 35 | AGCAAATACAAGATGACGTTAG | chr3LG5 | 104891818 | 38.498 | 0.045 |
| | | | AGCAAGTTGAGGCTAACCAGAT | | | | |
| | | | TACCATGAGGACAAACATCAGT | | | | |
| | | | ACGATCCAAGAGAAGATGGATC | | | | |
| | | | AGCTGTTGGAAA[[T/C]]AATGCTC | | | | |
| | | | GCGATTGCTCAAAGAGAGAGGA | | | | |
| | | | TTGAGGAGGAAGAAGCTAGGGY | | | | |
| | | | AAAAAGGAGATGGAGATCGTCC | | | | |
| | | | CCACCAAGAAGAGGCTGGTTCA | | | | |
| | | | GATAC | | | | |
| Ps03_205819517 | T/G | 36 | TTGCAACATTAGGGTTAGCGTAG | scaffold | 72342 | 47.175 | 0.044 |
| | | | AAGAATTTGGAGTAAAGGAATT | 00254 | | | |
| | | | GGGGATGAGTTGTAAATTTTAAT | | | | |
| | | | GGATAAGGATGARCACTAGGTG | | | | |
| | | | CCMAAAGTCT[[T/G]]AAAAGTTGT | | | | |
| | | | SAAGGWCTGYTGYTGRTACTAC | | | | |
| | | | TTCAGTGATGTGACAATGTCATG | | | | |
| | | | TTGTCTTCCATGTGCATTGTACT | | | | |
| | | | ATCATAGTACAAYACAAAATAT | | | | |
| | | | T | | | | |
| Ps03_206829164 | G/A | 37 | MCTCGGTCCAATCACAAGACCG | chr3LG5 | 30219372 | 47.340 | −0.042 |
| | | | ATMTTTCTGAAGGATCTTGAATA | | | | |
| | | | TAGGCRCACATGTKCCAGTCATA | | | | |
| | | | TGCGATATAAATCTTGAGATATA | | | | |
| | | | GTTCAAGCG[[G/A]]CCGAGGAAAC | | | | |
| | | | TTCTGACTTGCTTCTCARTTTTRG | | | | |
| | | | GCACATGCATTTCTTRTATTGCT | | | | |
| | | | TTKACCTTGGCGGGATCAACCTY | | | | |
| | | | AATACCCTTCTCGYTGACAA | | | | |
| Ps03_238101773 | G/T | 38 | TGTCTAAGCAATTTGCGGGCGA | chr3LG5 | 173063548 | 54.200 | 0.060 |
| | | | GAGCCTTTCCACCAATATGGYTG | | | | |
| | | | CCACATGTTCCTTTGTAGACTTA | | | | |
| | | | GGAAAGTACTAAGGCAATTTCA | | | | |
| | | | TATTCTCCCA[[G/T]]ACATYGTAA | | | | |
| | | | CATTGGAATTACTCTCCTTATTT | | | | |
| | | | TGTAAAGTTTTCATACAAGAAG | | | | |
| | | | GGTGTATCTGGGCGCCTGCTTTT | | | | |
| | | | GGACCTTTCTTGATTCCACTTCC | | | | |
| Ps03_241025997 | G/T | 39 | TTGATATTGTTGGGTGTTTTACC | chr3LG5 | 174636272 | 54.892 | 0.048 |
| | | | TAAGTCAYGTGCTCATTTMAAA | | | | |
| | | | TAAACTCTCTTATATGGAAGAGA | | | | |
| | | | TTCRTTKACCTTTGAAGAGGTTC | | | | |
| | | | AATCAGCCT[[G/T]]GTACTCTAAG | | | | |
| | | | GGTTTAAACRAACGAAAAGAGC | | | | |
| | | | ACAAGMTATCTTTTRTTGATGAA | | | | |
| | | | AGATTGTCCGTAAAAGGAAAAT | | | | |
| | | | TCACAAATAAAGATAGTAGGTT | | | | |
| | | | T | | | | |
| Ps03_481796573 | A/T | 40 | ACCGCAAAACAAACAAACACTA | chr3LG5 | 393751811 | 93.492 | −0.063 |
| | | | AGTCTCCCATCTGCGAGCAAAC | | | | |
| | | | AAGCCAATGTTTAAYYATTAGA | | | | |
| | | | ATGTGACATAAACATTCATTCAC | | | | |
| | | | TAAAATCAATY[[A/T]]ACCAACAC | | | | |
| | | | TCATTTGCTACTAAAAACTACGT | | | | |
| | | | AGCTTTGAGTTCTCCATYGCACC | | | | |
| | | | TRGAGATACGTAGGAGGGAGAT | | | | |
| | | | TCAACATCTCRTCAARCAYTATA | | | | |
| | | | A | | | | |
| Ps03_483314788 | T/C | 41 | GATGCAGTCATTTTGACCATGGA | chr3LG5 | 396332351 | 93.500 | 0.042 |
| | | | GGAAACTTCCTTGTACCCCATTT | | | | |
| | | | YGTCAAAGAGGAGYCGTGATGC | | | | |
| | | | AGATAAAAAATCTCCCATAAAA | | | | |
| | | | GAGGGACTCC[[T/C]]AAGTGAACG | | | | |
| | | | YGCCTAACTAGGGATGCTTCTTC | | | | |
| | | | AGGGGAGATAACTTGTTTCTTAT | | | | |
| | | | GAAAGCCTGACATCCTTCCTTTC | | | | |
| | | | CGGCWTGCTTGCACCCTTAAAA | | | | |
| Ps03_507346266 | A/G | 42 | GTTTCTTTCATCCACTTGATCAC | chr3LG5 | 417958980 | 98.180 | −0.041 |
| | | | TGGAAACACACGGTAGCTTTCCC | | | | |
| | | | TTCTTCWMCTGGCTCTYAATTTG | | | | |
| | | | TTCTMTAACWGAAACAACCTCA | | | | |
| | | | RAGAAAGTT[[A/G]]GGAAGCTGG | | | | |
| | | | AGAGTCCCTWTAAACATATCRA | | | | |
| | | | TCAACTATTTGTCAAGGATATGA | | | | |
| | | | GGYTGGACAYGAGTTGCAAGTT | | | | |
| | | | GTATCCACCACTGGRCATATTCC | | | | |
| | | | T | | | | |
| Ps03_511404191 | G/A | 43 | CTCYTATTTATACCAATTGAAAT | chr3LG5 | 421049387 | 99.800 | −0.048 |
| | | | AACCKCTTACAACGGCCTACTTA | | | | |
| | | | CCASATTGAGACAAGTGGCGCY | | | | |
| | | | CTGCATGCATGTAACTGCCCACT | | | | |
| | | | ACGTCCMAC[[G/A]]TATATCCTTA | | | | |
| | | | GCAGTTATATAAGAACARAGAA | | | | |
| | | | TTCCMTCCCTTATTTGAATTCAA | | | | |
| | | | ATCCTTTGTCAAACTAACACYAG | | | | |
| | | | TGACRCTTCTGTCGAAAACATC | | | | |
| Ps03_513771826 | T/G | 44 | CAAYGGCRGTACAATCATGTGG | chr3LG5 | 423551062 | 101.680 | 0.069 |
| | | | ATGAAAYAACTTCCACTTGAGG | | | | |
| | | | GAGACRATAGTGGAAGGACTCA | | | | |
| | | | CAYTCGAGGGGGAGTGTTKAGA | | | | |
| | | | TGGAAATRTGGA[[T/G]]ACTTGAG | | | | |
| | | | CATTTATGAGTGATAGAAYCCA | | | | |
| | | | CCCACYTATCATCTTAAGATTTT | | | | |
| | | | GGGTTGAGATGTGGTATMTTCCT | | | | |
| | | | CTCTTGTGGTCCTGAAGCATTAG | | | | |
| | | | TC | | | | |
| Ps03_531014546 | T/C | 45 | CACGCACTCCTGAGACTAGGATT | chr3LG5 | 827484 | 113.300 | 0.043 |
| | | | TGAGATGGATATCTCGCCCATCC | | | | |
| | | | AAGTTCTCGCCATTACTCAAAAC | | | | |
| | | | ACATCAAACCAATCAATTTCTTT | | | | |
| | | | TCTAGCCG[[T/C]]CGTGCGATTAA | | | | |
| | | | CTCTTAAACCTTTTCTCGCCGCC | | | | |
| | | | GTGCGATTGACCCTTCAATCCTT | | | | |
| | | | TTCTCAAATGAAAGGTATTTTGT | | | | |
| | | | TTTAAAATGATGCAAGGCAA | | | | |
| Ps03_531232613 | G/A | 46 | GGAAATTAAGAATATCAGCCTC | 3 | 425962517 | 113.300 | 0.042 |
| | | | AAGAAGTTTCATAACCATCCAA | | | | |
| | | | GAATAAATACACCAATAAAAAG | | | | |
| | | | GAGTGAAAATGAAAAATGATGC | | | | |
| | | | CAGTAGAATTTA[[G/A]]GAGAAAG | | | | |
| | | | GGTTTTCAAAGAAAGGAGAAGG | | | | |
| | | | GGAAAAAAGAAACCTGAACATA | | | | |
| | | | TCAGGGCGTTGATCGTATTCAAC | | | | |
| | | | ATGCTGCCCATTGGACGTGTCTC | | | | |
| | | | TCC | | | | |
| Chr3-4 | T/C | 47 | ATGCATTGGTTTATTGATTGAAG | 3 or 5 | 425968088 | 113.300 | 0.046 |
| Ps03_531239107 | | | AATTATGAATAGGTAATCATATG | | or | | |
| | | | CAAAGTAATCAACTTTTGGACCC | | 563531992 | | |
| | | | CTTTGCTTCTAATTCTCAACCAT | | | | |
| | | | AATGACAA[[T/C]]CAAAATCCTAC | | | | |
| | | | AAAGATTCAGATGACGATAGTA | | | | |
| | | | TTCTTGATTGTCACCCTCTAACC | | | | |
| | | | TCTGAAACATGATGCAATGGGA | | | | |
| | | | TGGATGATGCATTGAGATTGAG | | | | |
| Ps04_9648139 | T/C | 48 | TCCAGAAKAAGAGACAATGGCC | chr4LG4 | 5972665 | 2.887 | −0.047 |
| | | | AATCATATATTGACATGCTATGA | | | | |
| | | | GTAAACATACTGAGAATGATGG | | | | |
| | | | ATTTAACCAACTYACCATACCTG | | | | |
| | | | CAAAATAGAG[[T/C]]RGCGAACCT | | | | |
| | | | TTCCACGAARCCAACTTGTTTAT | | | | |
| | | | AATTTTATATGCAATAAACTGCA | | | | |
| | | | AGAAAAGCTTMTTTGGTTTTCCT | | | | |
| | | | TTAAAYARAGGAACACCAAGAT | | | | |
| Ps04_26115694 | T/C | 49 | CGTGCCCAAGTGTTTTTTCACGC | chr4LG4 | 18486554 | 10.500 | −0.098 |
| | | | CGYGATAACTCTTTTATTTTAAA | | | | |
| | | | ATCAACCAACCCACAAWTTTTTT | | | | |
| | | | ACAAAGAACTATGYAGCTTTGA | | | | |
| | | | ATTCTTCAT[[T/C]]GCACCTAGAG | | | | |
| | | | ATACATAGGAGCAYGATTCAAC | | | | |
| | | | ATCTTGTCAAGCATCTTAATAAA | | | | |
| | | | AAATAATAKTTTTCCCTTTCCTA | | | | |
| | | | TAAACATGTCATAGTAATTATG | | | | |
| Ps04_106176050 | T/C | 50 | GAAGYTAGGGCTCCAGTGATGT | chr4LG4 | 83578489 | 34.352 | −0.050 |
| | | | GTTGGCGTGCCTCYARCCATCTG | | | | |
| | | | ATCRTGTTAAAACRTTTTAATCT | | | | |
| | | | CAGYCTTTGTTTTRWTTGACCCA | | | | |
| | | | GCGTACGCR[[T/C]]RCATTKACTA | | | | |
| | | | AGGACCAGCGTGGATGACATGC | | | | |
| | | | ACGTGGCCATCAGATATGTCACC | | | | |
| | | | TCAAWTAATGAAGGAGTTCTGA | | | | |
| | | | TGGCCCTTGTTTTTCTATTTTCT | | | | |
| Ps04_119030031 | C/T | 51 | TTATTGAGGTAAATTATTATTCC | chr4LG4 | 94928513 | 37.200 | 0.036 |
| | | | ATGATAACATGATTTAAGCTTCT | | | | |
| | | | ACTCACAATGCATTGTGTTTCTC | | | | |
| | | | GTGTTATTTACACAAGGGTGGA | | | | |
| | | | AACTATATA[[C/T]]GGATGACCCA | | | | |
| | | | TGTAAAATTTCTGTACAAAATTG | | | | |
| | | | GTTGTGTCAGKAGAATCTGTGTC | | | | |
| | | | CTATTATTGGAAAATGGGAAAA | | | | |
| | | | ATTCCCTTATATTACCAAAACA | | | | |
| Ps04_126746363 | T/A | 52 | TTGTTTTTGCGAGAAATAAATTG | scaffold02127 | 47049 | 39.469 | 0.047 |
| | | | GTTCTAAATTCAATAACAAGAA | | | | |
| | | | AAYGATTAGGTTTTAYTTTAATC | | | | |
| | | | CCTTGTATTGTCCCTTGTTGATG | | | | |
| | | | TTTAATGGA[[A/T]]AATATGAATG | | | | |
| | | | GTAATGTCATTATATTTAACAAG | | | | |
| | | | GTAATATTAAAAGATATCCACTC | | | | |
| | | | CACGAGGATTGCGATTTAACAA | | | | |
| | | | GGTAAAAATMATTCGAATTAAA | | | | |
| Ps04_133748675 | C/G | 53 | AGTGAAGTGTTGTCTGAACTGGT | chr4LG4 | 109277412 | 41.086 | −0.033 |
| | | | GGAAGTTTCAAAGGCTCTTCAA | | | | |
| | | | AAGACTAGTACTGCTWGTATTA | | | | |
| | | | CCAGAAAGAGGAATATAAATTA | | | | |
| | | | GTTGATCAAAA[[C/G]]ATTATCTA | | | | |
| | | | AGGAGAAGGAAGTTGACAAGGC | | | | |
| | | | AGAAGAGGYTCACAATGAAGAT | | | | |
| | | | GAAGAGAAAGARAAGCCTGCAA | | | | |
| | | | ATAATGGAGATTCTAATAGCTCA | | | | |
| | | | AAT | | | | |
| Ps04_140768543 | A/G | 54 | CGGATTCCATCAGCGTCGTAAGT | chr4LG4 | 117043426 | 43.095 | −0.028 |
| | | | TTGGCAATCTCATCCTTCAGTTC | | | | |
| | | | TCTATTTTCTTGCTYTAGATGTTC | | | | |
| | | | CATTCTCTTTGGTTGATCAGCAG | | | | |
| | | | TGTTATA[[A/G]]CGGTGAGTCAGC | | | | |
| | | | TTKGCTGATGCACAAAAGAAGA | | | | |
| | | | ACTGATAAGACATCTGGMGGGA | | | | |
| | | | GAAACCTGTCATRCAAATGATG | | | | |
| | | | CATGAAATGCAATGTTTTTTTA | | | | |
| Ps04_196413843 | G/T | 55 | CACGTGGCGTCTATAACTAYAGT | chr4LG4 | 163719335 | 56.532 | −0.043 |
| | | | TATTAATTCTGCTCCAGTTGCTC | | | | |
| | | | AAGCARCACCGAGTTATCAACC | | | | |
| | | | GCATTTCCAGCAACACACGAAT | | | | |
| | | | CAACAGAATC[[G/T]]TGCCCAGAG | | | | |
| | | | GCCTGTACAATTTGACCCAATTT | | | | |
| | | | CGATGACTTATACAGAGTTGTTT | | | | |
| | | | CCTACTTTAATTCAGAAGAATTT | | | | |
| | | | TGTGCAAACAAGAACTCCACCG | | | | |
| Ps04_198084088 | A/C | 56 | AAATTTGAAGTCCATTGGATTAA | chr4LG4 | 165487268 | 56.661 | 0.060 |
| | | | AATCAAGCATTTCGCAAAGGAA | | | | |
| | | | AGAGTAAGGCAAGGTCACAAAT | | | | |
| | | | GCACCTATGGTCAAAGATTATA | | | | |
| | | | ACACCAGCAAA[[A/C]]ATCAAGCC | | | | |
| | | | ATGAACAACTTTCAAACTTATGA | | | | |
| | | | TCATAAAAAACTAGACATTCTA | | | | |
| | | | AGGATCATTATGCAAAAAATTG | | | | |
| | | | GGAGTGATATGATGCATTTCTCA | | | | |
| | | | AT | | | | |
| Ps04_198169869 | G/A | 57 | ATCRTCTTTCAGGTTGTCTCCTT | chr4LG4 | 165597701 | 56.668 | 0.057 |
| | | | AGTGAGCACAGAGAAAGTGATA | | | | |
| | | | CACCACCCAGATCCCCTGAGAG | | | | |
| | | | TCTGGAGTTTGGTCCTAATCTTC | | | | |
| | | | TGGCTCYATA[[G/A]]TGAGGCTGG | | | | |
| | | | ATCTAGCATYTGTGTTATCAATT | | | | |
| | | | ATATGGTTGTCATCCCTCGTGTT | | | | |
| | | | GATTGACCTAAAATCCCTTATAG | | | | |
| | | | ATTGATAAATTGGTATGTTGTC | | | | |
| Ps04_256098157 | G/A | 58 | CAGCTTCCTCAGTGRTCTATATT | chr4LG4 | 218884465 | 70.620 | 0.028 |
| | | | ATTAACCACACTTGCTAAGACAT | | | | |
| | | | TGATGGTGATATTATTGAAGTTA | | | | |
| | | | TTGAGTTGCTCAATTCTTGATTS | | | | |
| | | | AAAACCCA[[G/A]]TAGTTGAGATT | | | | |
| | | | GTAGGTCAATCCAACTAAGAGA | | | | |
| | | | TTTGACCAGAATGTTTAATTACT | | | | |
| | | | ACGGGGTTATACTTAGAGTAAA | | | | |
| | | | AACCATTTAAGGTTTGAATTAT | | | | |
| Ps04_263312773 | A/G | 59 | CCTTCTCACAATGAKCCMTTGAT | chr4LG4 | 228522691 | 71.900 | 0.080 |
| | | | CAACATCAARTTARWTTCAAAA | | | | |
| | | | TCTCRCCATTTTCAAATCATTTCT | | | | |
| | | | CACATATGCGAATTAAACCTCG | | | | |
| | | | ATCAACATC[[A/G]]ATTGRYTTTT | | | | |
| | | | CTTAAATCARAYGTAATATCAA | | | | |
| | | | ATTCTTTTTCTTAAACAAAYGCG | | | | |
| | | | AAAGAATAGGACYGTTGAARTC | | | | |
| | | | CAGTATTCCAGTGGAACCCTCGA | | | | |
| Ps04_284358817 | C/T | 60 | CAATTGAATTGGAGACTCCTCTT | chr4LG4 | 247901046 | 74.417 | −0.028 |
| | | | AAAATTGAATGGAGGCGCCAAT | | | | |
| | | | TGGATTGGCGCCCCTATGTAGGA | | | | |
| | | | TTTAAGAGRAGGCGCCAATTCA | | | | |
| | | | ACTGGAGCCT[[C/T]]AGTRTAAAA | | | | |
| | | | TGCCTTTTTTTKGGTAAATGGTA | | | | |
| | | | TACGTGATAGATAAATTTGATAT | | | | |
| | | | ATGACCAATTGATATTAATAAA | | | | |
| | | | AATTGTCGTTTACACAAAGAAA | | | | |
| | | | C | | | | |
| Ps04_327258970 | G/C | 61 | TGAATACCGATCTCAGAAGGCA | chr4LG4 | 352524054 | 75.504 | 0.043 |
| | | | ATTAATSGTAGTATCTTAGCTGA | | | | |
| | | | CCATTTGGCTCACCAACCGATTG | | | | |
| | | | AAGATTACCARTCAGTGTAGTAC | | | | |
| | | | GATTTTCCT[[G/C]]ATGAAGAGAT | | | | |
| | | | CTTGTACATGAAAATGAAATATT | | | | |
| | | | ATGATGAATCGTTGCTTGAAGA | | | | |
| | | | AGGGCCAGAACCTGGTTCCCGTT | | | | |
| | | | GGGGCATGGTATTTGATGGAGT | | | | |
| Ps04_347955117 | G/A | 62 | CAATAAAAAATCRAGAAATTAT | chr4LG4 | 363782042 | 75.504 | 0.037 |
| | | | TCTAGGACACTTCATGTTTTTTTT | | | | |
| | | | AAATATCACATATTCCCTCAGTT | | | | |
| | | | TTTGAATATAACCGGGGTGAAA | | | | |
| | | | ACAAAYTCG[[G/A]]GACATGCTTC | | | | |
| | | | TAATCCCATCAAATGCAGTTTAT | | | | |
| | | | GTTTTTATTTCTGTAAAAGTGTT | | | | |
| | | | GTCTTTATTTTTGTTTTACTTTTT | | | | |
| | | | ATTTATAAATTAAAGGATKT | | | | |
| Ps04_374415380 | T/G | 63 | GCARCAAGAAAAAGTAAGTATT | chr4LG4 | 285377934 | 78.167 | 0.032 |
| | | | TTGATTTTTATCGTCTYCATAGG | | | | |
| | | | RATTAATGTGATATCAATGTCGT | | | | |
| | | | TCAACGGTCTCTACGATTTTAAT | | | | |
| | | | CTTGGRTTT[[T/G]]TAAGATTTGA | | | | |
| | | | ATTTAAAGGTAAGCTATGTAAA | | | | |
| | | | ACGGATAAATAAATAAATAAAG | | | | |
| | | | CTTCAGAGAGATATAARAGTTTT | | | | |
| | | | ACAAGATWGAATTTCATCTACC | | | | |
| | | | G | | | | |
| Ps04_378090615 | C/G | 64 | TTTATTCCATCTCATTTTATAAAT | chr4LG4 | 389689436 | 78.634 | 0.032 |
| | | | TTAGAACAGCAAATAAGCAATG | | | | |
| | | | TATGGAAACTATTGTAGGACCTA | | | | |
| | | | GCATTAGAGTTCCCAAAACTCG | | | | |
| | | | ATCCTGCTG[[C/G]]TCTTGATCTTC | | | | |
| | | | TTTCGGTAAGTTAACTGCCCCGT | | | | |
| | | | ACACATTACTGTTTACGTTTCTT | | | | |
| | | | CGCCTAAATTACTCARCTCTTTT | | | | |
| | | | ATTGATCTACTTAAAGTTTA | | | | |
| Ps04_386293806 | T/A | 65 | TATCTGTYTAGGTARGAGCTAGA | chr4LG4 | 2191851 | 80.040 | −0.034 |
| | | | AAGAAAACARYCAATCAAGACT | | | | |
| | | | CAACCCAATRAGGACATAACTC | | | | |
| | | | AAKGKGAGTAATTCCRTCCAGA | | | | |
| | | | AAATTTGCTGG[[T/A]]GTGGAAGC | | | | |
| | | | AGAATTAGMAATCATMCACGAS | | | | |
| | | | GAAACAAACTCAGTRGGGAAAC | | | | |
| | | | AGAGAAAGGKTAAAGTCTTTTC | | | | |
| | | | TRCWTAGGGGCTGACACTCTAC | | | | |
| | | | AGTT | | | | |
| Ps04_434414625 | G/A | 66 | TACRGAAWAGTGGCGACTCAGC | scaffold00706 | 145804 | 94.142 | 0.057 |
| | | | TAGGGARTAGYCCTCAGTGGKT | | | | |
| | | | TTAGCCTATCTTTGTTTATGTGT | | | | |
| | | | ATATATTATGTTCACTRTTGTTT | | | | |
| | | | ATTATTGTTT[[G/A]]TTYTGTCTGG | | | | |
| | | | TRCTTGGTGATCTCTATGTGGTG | | | | |
| | | | AGATAAGTCTTATAMCCRTACTT | | | | |
| | | | GAGAKMTCTTATTGTAGGAGGG | | | | |
| | | | TGGTATAGTCTTGTTTGGCTTA | | | | |
| Ps04_445153308 | C/A | 67 | ATTATTATCATGTGAGGTTTGAT | chr4LG4 | 374997960 | 99.000 | 0.073 |
| | | | ATATAACAGGATCTTATGCTGCT | | | | |
| | | | TGTCAAGAAATTTGGATTAGATA | | | | |
| | | | TGTGAAGCAAGATATGAATGTC | | | | |
| | | | GAAGTGAAG[[C/A]]AACCTTTGGT | | | | |
| | | | GTTGCARATTAACAACAAGTCAT | | | | |
| | | | CTATGAATCTCGCAAAGAATCC | | | | |
| | | | AATTCTACATGGGAGAAGTAAG | | | | |
| | | | CACATCGAGCCTAGGTTTCATGT | | | | |
| Ps04_457675265 | A/G | 68 | TAGAGAAGTTTGTGAAGAAAAT | chr4LG4 | 418184353 | 108.045 | 0.061 |
| | | | AACGAGATTATTCAGTTACAAC | | | | |
| | | | AACAACTACTTTTAGATATCACA | | | | |
| | | | AACACAAATGTGATGATACATA | | | | |
| | | | RCATTGCATCA[[A/G]]GGGAAATG | | | | |
| | | | ATGATCAATAATCTTTATGGCAC | | | | |
| | | | TAAAGTACCACTACAACATTTTA | | | | |
| | | | TACTTTAGRCAACRGAAGAATA | | | | |
| | | | GGCAACGATTGAAAAAACGTCA | | | | |
| | | | CC | | | | |
| Ps04_463538432 | C/G | 69 | ATAATAAATTCTTGAAGGGACTT | chr4LG4 | 444278355 | 111.826 | −0.045 |
| | | | TGATACGTGCATTAATTCAAACA | | | | |
| | | | AAACAAAAYTTCTGGMAGTGCC | | | | |
| | | | ARACATTAGAGGGACTTYAGGG | | | | |
| | | | RGGTCMTCTT[[C/G]]ATTTTTTMG | | | | |
| | | | ATAAAAATGGATGTTGGACTTA | | | | |
| | | | GACATGTAGGRAACATGTATCC | | | | |
| | | | AAGAAAATATGGGGTAGTTGAA | | | | |
| | | | AGTTAGTGGACTTRCACTAACAC | | | | |
| | | | CC | | | | |
| Ps04_464099084 | T/C | 70 | CTGWGTARTGGGATAATTGAGA | chr4LG4 | 445125850 | 111.900 | −0.086 |
| | | | AAGGCTTTGAGRCAAGGGTARG | | | | |
| | | | AGGTGTACRCAYTATCCTTCYGG | | | | |
| | | | ARYCTCCCCTATTCYAACTCAAG | | | | |
| | | | TGCACTRCCT[[T/C]]CRWTTTAAA | | | | |
| | | | CGCCTTTTGACCRGATCTTTCRC | | | | |
| | | | CCAACTTCCATTTCCTAGTCCAT | | | | |
| | | | AACGTGCATTTRTGAGTATAACG | | | | |
| | | | TGTAATAAGTCAACAAATAGTG | | | | |
| Ps04_467088335 | G/A | 71 | ATTGCARACATCATGACGAAAG | chr4LG4 | 420833970 | 111.900 | 0.039 |
| | | | AAGTGCATGTCAAAGTGTYMAA | | | | |
| | | | GAAGTTAAGATCTATGATGAAC | | | | |
| | | | GTAGATAGCTTAGACAYAATRA | | | | |
| | | | ATTAGGTGGTGT[[G/A]]TTGAGAA | | | | |
| | | | TGTAATTCGGYGTGWCGATGCA | | | | |
| | | | GCAATTTCGACACARTCAAGGA | | | | |
| | | | AATGCATGRTGTGTCAAAKTRTA | | | | |
| | | | ATAGTTGTTGAAAGAAGTGTGTC | | | | |
| | | | TTY | | | | |
| Ps05_5262178 | T/C | 72 | GATGCTGAGCAGTTGCGTTCATC | chr5LG3 | 362278 | 0.944 | 0.040 |
| | | | RCCAAATGAGGACGACTCTGAT | | | | |
| | | | TGGGAGGATGAAGACCCTGATG | | | | |
| | | | ACGAAGGTATCATTTATGTGGA | | | | |
| | | | ATAGCTGTCCC[[T/C]]GAAAGCAT | | | | |
| | | | CAGTGTTGGTAAGTATGTTTTGA | | | | |
| | | | TGAGCTTATGTCATGAAGATGAT | | | | |
| | | | TAGTTCAGTTATACTTTGTATAT | | | | |
| | | | TTGAGTTTGGTTYGCTTTAGAAT | | | | |
| Ps05_17115394 | C/A | 73 | GATTTAAAGACAATAATCTTTAT | chr5LG3 | 178039871 | 3.206 | −0.041 |
| | | | TCTCAGTTGGTCGGCAACATATC | | | | |
| | | | TCGAGGGGATTTAAATTATATTT | | | | |
| | | | TTCACGAAGCTWAACGATCTGA | | | | |
| | | | TAATGTAGG[[C/A]]TCCAATAGCA | | | | |
| | | | CAAAGYGTGGATGCACAATTGT | | | | |
| | | | GAAAATATATGGTMTTCCATGT | | | | |
| | | | GCCTGTGTTATTGCAAAAAASGT | | | | |
| | | | GAAACTTGGTAGCCTGATAAGA | | | | |
| | | | A | | | | |
| Ps05_23320549 | G/A | 74 | AAAYCAACTAYAACCTGMCAAG | chr5LG3 | 20543180 | 5.019 | 0.035 |
| | | | YTTGGATACTCAGAGAAAMGAA | | | | |
| | | | AGGGGAAAAAACATACCATGAT | | | | |
| | | | AGGCTACTAATTTCTTCTTCTCTT | | | | |
| | | | CTGAGACAAC[[G/A]]GCGTTTTTC | | | | |
| | | | TTTTCTGGTCCAGCAATGATCCT | | | | |
| | | | TTCAAGTGCATCAGATATCTCAT | | | | |
| | | | CTTTACTTATTTCCTTGAGCTCG | | | | |
| | | | CGCCTGGCTGCAAGAATAGCAG | | | | |
| Ps05_48702172 | A/G | 75 | TCATGAAGATCACCATGTAGAA | chr5LG3 | 35018191 | 9.664 | 0.132 |
| | | | ATACATCATGGACATCTAGCCG | | | | |
| | | | ATGAATAGGTCGYGATTTGGAC | | | | |
| | | | AGAGCAATGGTGAGAAYGGAGY | | | | |
| | | | GAATAGTGGCCG[[A/G]]TTTGACC | | | | |
| | | | ACATGACCRAATGTCTCATTATA | | | | |
| | | | ATCTACACCTGAAATATGTGACC | | | | |
| | | | TTCCATCACCTACAAGAYGAGCT | | | | |
| | | | TTGTAGGCTGAAAGCAACCATT | | | | |
| | | | AG | | | | |
| Ps05_51336818 | C/T | 76 | CCTCTTGGTAGTTAAATACATGT | chr5LG3 | 37349400 | 10.300 | −0.044 |
| | | | GTCAAARTTAACTGAAYGTAGG | | | | |
| | | | ACTGAAAGTCATCAAGGAGAAG | | | | |
| | | | GGGTGGGTTAAAGTTAAATTTTG | | | | |
| | | | AGCCTTATAT[[C/T]]CTTTTGTTCA | | | | |
| | | | AAAAAAGCATGAACCRGACCAT | | | | |
| | | | GTTACAAACTTAAAAGACCTAA | | | | |
| | | | TTAAGRCATGATTTATTTTGAAA | | | | |
| | | | GCATATTGTTGAAGCGATAAAG | | | | |
| | | | C | | | | |
| Ps05_53552642 | G/A | 77 | GTGCATGRAGAAAACRAGGAAG | super- | 39703 | 10.928 | −0.053 |
| | | | AAGGATCAGAAGGCGTTGTTTA | scaffold888 | | | |
| | | | ATWTTCATAAGCGTGTRAATAC | | | | |
| | | | AAATGTGTTTGAGAATATCAYTA | | | | |
| | | | ATTCAACGACG[[G/A]]CGAAGRTG | | | | |
| | | | AAGGTGTGAAAAACAYAAGAAA | | | | |
| | | | TGGGGGTTTAAATTGGGTTTTAT | | | | |
| | | | AAGCAAAARYTTTTTCYAAAAC | | | | |
| | | | AAGAACACYACAAACAAGTTAA | | | | |
| | | | AAA | | | | |
| Ps05_54722636 | C/T | 78 | GTWGAACCTCTGACCTTAGRGT | chr5LG3 | 39908055 | 11.277 | 0.030 |
| | | | CATGGGGTCAACCCCCTTACCAA | | | | |
| | | | CTGAGCCAAGCGCTCTTGGTGAC | | | | |
| | | | AAAAGCGTGCTTCAATGAAATA | | | | |
| | | | AATAATAAAA[[C/T]]AACAACAA | | | | |
| | | | AAATAAARCASRCGTGTGATTCA | | | | |
| | | | GGCAATSAGAGTGAGACAACAA | | | | |
| | | | AAGTTTTCAAATKCAAWCGCGT | | | | |
| | | | GAARGTGACTTCGTCTTCAACCT | | | | |
| | | | TT | | | | |
| Ps05_134772954 | A/C | 79 | TAGCTTGGTTACCACTTTGGTTT | chr5LG3 | 96025751 | 34.203 | −0.056 |
| | | | TGGTTGTTTCTCCAACGAAAATT | | | | |
| | | | CGGATGMTCCTTCCATCTCGAAT | | | | |
| | | | TCTAGGTATTGGAATAAGGGTTA | | | | |
| | | | TTTTTCTG[[A/C]]AAATTTGCAAAT | | | | |
| | | | TGGGYCTCTTCACTTACCCCTTC | | | | |
| | | | CAACAKACACCTTTCRTTTTCAT | | | | |
| | | | GTCCTCCTCCATAAAAGTCACAC | | | | |
| | | | TAAGTGATTGTACCYGGCT | | | | |
| Ps05_139126831 | A/G | 80 | CAAACACAATATACACAATCAC | scaffold00462 | 12104 | 35.269 | −0.078 |
| | | | AATATATCACAAAATATGGTCC | | | | |
| | | | AAATGGACAAAGGGAAAATGAC | | | | |
| | | | ATTAACATAAACAACTTGAATG | | | | |
| | | | GTATGAATAATG[[A/G]]CAAATAA | | | | |
| | | | ATAAAGCTTAAAAATTAAAGTG | | | | |
| | | | CATTAAAAGTAAAGGCTTGAAA | | | | |
| | | | TTAAATGTTAGTTGTTAGTTGAT | | | | |
| | | | TAGAAATTAGTATTGCTTTTGAT | | | | |
| | | | TTA | | | | |
| Ps05_173144250 | A/G | 81 | ACTGTACAAAYAATRTGGTTGG | chr5LG3 | 132883215 | 42.800 | −0.027 |
| | | | ATATGAAGCTTATGTCATAGGTC | | | | |
| | | | TCRAAGCTRCTATYGACCTAAGG | | | | |
| | | | ATCAAAACTCTGGAGGTGTACA | | | | |
| | | | GAGACTCAAC[[A/G]]CTYGTTATC | | | | |
| | | | TGTCAAGTAAAAGGMGAATGGG | | | | |
| | | | TAACTCRTCACCCAAATTTGATT | | | | |
| | | | CCTTATCATGATCATGTCTTAGA | | | | |
| | | | ATTGATGAAGAATTTYGAAGAA | | | | |
| | | | A | | | | |
| Ps05_175640373 | G/A | 82 | CCTTCTRGATCAGCTRTTRTTRC | chr5LG3 | 134409547 | 42.923 | 0.036 |
| | | | CATCAATGCAACATTRGCTYGTT | | | | |
| | | | CCTCGTCAGACTCWTCTTCTTCT | | | | |
| | | | TCTGATTYTGAGTCGTCCCATGT | | | | |
| | | | AGCCMTCA[[G/A]]CCCCTTATTRC | | | | |
| | | | CTTTAGAAAAATWCTTCTTAGG | | | | |
| | | | CCYTCTGTCMTTTTTTAGCTTAG | | | | |
| | | | GACAATCATTTTTATAGTGGCCT | | | | |
| | | | KRCTCCTTGYACTCAAAGCAA | | | | |
| Ps05_217776534 | G/A | 83 | TTGRGACTGACACTAAAAAAAT | chr5LG3 | 24130766 | 53.100 | −0.086 |
| | | | GCAAAATTACCTTTTGTTGCTAT | | | | |
| | | | AGAGCAAGAAGRGGTTGGAGAC | | | | |
| | | | TCGAGGAGTYTGTACAKYCTGT | | | | |
| | | | CTAGTTGCTCC[[G/A]]TAGTGAGT | | | | |
| | | | GAAAACTGAGACRRATGRGATT | | | | |
| | | | RWTGTCCTTGATCAGAATTACC | | | | |
| | | | AGCCTAAAATGCACGRCCAYTTT | | | | |
| | | | TCTTCTTMCAKTTAKGAGGTTTG | | | | |
| | | | CC | | | | |
| Ps05_265627777 | A/G | 84 | GGTAGGGATACGCAATTYTGTTT | chr5LG3 | 217510948 | 65.524 | 0.030 |
| | | | CTTGCTGAACCATGAAATCAAAT | | | | |
| | | | TGTTCCCAAGAAGAAACATCCTC | | | | |
| | | | CRGGGGTACTCTTCCTATCATYA | | | | |
| | | | ACACTACC[[A/G]]GYCCAATCRAC | | | | |
| | | | ATCATAATACCCCACTAATATAG | | | | |
| | | | AATTTGAATCACGTGAATACAA | | | | |
| | | | CATTCCATACTCACTTGTGCCAT | | | | |
| | | | TSACATACTTTAGARTCCTTT | | | | |
| Ps05_268032915 | T/C | 85 | GTCTTCTTATCTTTCAAAGATGC | scaffold02449 | 84199 | 66.184 | 0.118 |
| | | | CCCASACGGATAAATCTGACTTT | | | | |
| | | | GGGRAAAACACTTGATATCAAA | | | | |
| | | | ATACCATGGCTTTTCATCTTTGA | | | | |
| | | | TCTCWTCAA[[T/C]]AACAAACACA | | | | |
| | | | TGAGCTGCCTATCAAGACGCATC | | | | |
| | | | ACAGTCAAATTGRRAACTTCATT | | | | |
| | | | CYAGAACTTCACCATAATTATTG | | | | |
| | | | AAACCAACGTTGCAAGAGCAT | | | | |
| Ps05_277838646 | T/C | 86 | TTCAAGGATCCATGCTTGTGAAT | chr5LG3 | 228797264 | 68.450 | −0.027 |
| | | | AGAGGATTGAGTGTTCTCCAAA | | | | |
| | | | GAATGACTTAAACAATTAAAAA | | | | |
| | | | GCAAAATCCCACTAACTTCTAAT | | | | |
| | | | TAAATAACAT[[T/C]]TGACTAACT | | | | |
| | | | TTTAATTTCAAGCCATTTACTCT | | | | |
| | | | TATGCACTTTAAATTCAAACCTT | | | | |
| | | | TTATCATTTTCCATTATACATAT | | | | |
| | | | CATTTGACTTGTTGATGTTTAT | | | | |
| Ps05_284520856 | A/G | 87 | AGGACCARAATCAGCTAACGTC | chr5LG3 | 234213508 | 69.641 | 0.040 |
| | | | TATAAAYYTCTAGGATGCAATT | | | | |
| | | | ATAATTGTTCCCTAAAATCAACC | | | | |
| | | | AACAAATCGGTATTTTCTATCAA | | | | |
| | | | GAACTACGRA[[A/G]]CTCTGATTT | | | | |
| | | | TCTCATTGTACTATGAGAATATG | | | | |
| | | | TAGGCACGAGGRTTCGAATCCTC | | | | |
| | | | GGAGAGCACACCAATTAAAAAC | | | | |
| | | | TTATTTTTCCCCTTTTATCATTG | | | | |
| Ps05_289702502 | C/T | 88 | CATCMAAAATGCAACTTACAYG | chr5LG3 | 236504935 | 70.630 | 0.055 |
| | | | ACCWTACSTAAGCAAGATTCAA | | | | |
| | | | CATCGTATAACRTACMACACCC | | | | |
| | | | TAAAATACCAATGCATGCAGTC | | | | |
| | | | ATATGGAAACAT[[C/T]]TAACAAC | | | | |
| | | | ASCGAACCCACTTCGTTAAGAAT | | | | |
| | | | GAAAAACTAAACCAAACACAAT | | | | |
| | | | TATATTACTCTTAACAAGACATA | | | | |
| | | | ATGCACGTRCAAAAYGAGTTAT | | | | |
| | | | CTG | | | | |
| Ps05_290623435 | G/T | 89 | CTGAWGTTAGTTTGTCCATCAAC | chr5LG3 | 239060496 | 70.890 | −0.056 |
| | | | TTACAAAAAAGGTTAATGGTGC | | | | |
| | | | ATTATGGTTCCAAGAAAGCATG | | | | |
| | | | GAGTKATTWAGGGAGAGTAGGA | | | | |
| | | | GTGYTTTTCCK[[G/T]]AGTTTAAG | | | | |
| | | | GGCCTTGCTTMTGGTGTTTAGAT | | | | |
| | | | AGAKTAAGATCAAASCAAAATT | | | | |
| | | | TATTTACAAAGCTCCAAGCAAA | | | | |
| | | | AGTGGTTGAGAAAAGTGATCTTT | | | | |
| | | | AT | | | | |
| Ps05_320108884 | T/C | 90 | CCCCAATCTTAWTTTTTYCTCCG | chr5LG3 | 268153046 | 78.211 | 0.037 |
| | | | WGGCCCATGCTGAAAAATATCT | | | | |
| | | | CAAACTAGTGGATTACCATATTG | | | | |
| | | | TGAGGGAAAGAGAATTCACTTT | | | | |
| | | | GGATAATTTA[[T/C]]AAGGTTTTR | | | | |
| | | | GAGAAGTGGGAGAATTTTTGCA | | | | |
| | | | ATAAAGGTGTTGGGTAAGTTTA | | | | |
| | | | AACAATCTAATTCATGACWYAA | | | | |
| | | | ATAAGAGTATCAGACTAGAATT | | | | |
| | | | TTA | | | | |
| Ps05_337541850 | C/T | 91 | ARAAATGAATCCACTRGGGACT | chr5LG3 | 2288318 | 80.900 | −0.075 |
| | | | CTACTGATGGGGAAAACAGGGG | | | | |
| | | | TACTTTTRCCGGGKATTGGGCAA | | | | |
| | | | GAAGTAACAAACTGCAAACTAA | | | | |
| | | | GAAGAATATTA[[C/T]]CAGTTACT | | | | |
| | | | SGGTAATAAACTCTTAGGAGACT | | | | |
| | | | TAGAGYATCKATCTAGGCAAGA | | | | |
| | | | GCTAGAAAGAAACRATYAATCA | | | | |
| | | | AGACTCAACCCAATGAGGATAT | | | | |
| | | | AAC | | | | |
| Ps05_338232797 | C/T | 92 | RAAGATGCAAAAGTYGAATMAT | chr5LG3 | 278088295 | 81.296 | 0.040 |
| | | | CCATCCTAAATCTCTTGAGTATC | | | | |
| | | | TCATTGACATACTTTCTTTGATG | | | | |
| | | | TAGCATCATACCTTGCTTYGACA | | | | |
| | | | TTTRAAATT[[C/T]]CAYGCGTAAG | | | | |
| | | | AAATAARACAAGTTTCCAAAAT | | | | |
| | | | CCGACATATCAAATTCCCTCATC | | | | |
| | | | RCCAGCTCTTTGAACTTCGWCA | | | | |
| | | | AGTTCTTCRAGCTATTTCCAGTT | | | | |
| Ps05_352490739 | C/A | 93 | AGGATCCCCTAGCACAAAAGCA | chr5LG3 | 84459019 | 86.015 | 0.025 |
| | | | CACACACATCAAAGAGTCTTAT | | | | |
| | | | GAAGCAAATCAAGAATGGACAA | | | | |
| | | | GAGTTAGTTTAGAGATTTGGTCC | | | | |
| | | | TTCTAATCCAT[[C/A]]TTCAACATT | | | | |
| | | | AGGCATTTTACTCCAATTTTGCA | | | | |
| | | | TAGGGAATATCCTAGAAACTAA | | | | |
| | | | GTCCATTTGTCCATTTMTTTGCA | | | | |
| | | | TTTGGTCCATAACAATCAAAACA | | | | |
| Ps05_358014672 | A/G | 94 | RTAGCATCAAGTACACCACTCYT | chr5LG3 | 306598116 | 86.878 | 0.108 |
| | | | ACTRCTACCRTTTTACTGTCTAC | | | | |
| | | | TCATGATGATGATTCTATGGTTA | | | | |
| | | | AKCWAGGGGGAAAACAAAAAA | | | | |
| | | | AATAGGGTTY[[A/G]]GGAGTTGTT | | | | |
| | | | RCGGAGGAGACTGRAACTTTTGT | | | | |
| | | | AGATGAGAGTGTGTATGCAAAT | | | | |
| | | | CTTTCCATGTGATCAWCGAAGA | | | | |
| | | | GATATTTCCCACYCAGGAGGCA | | | | |
| | | | GT | | | | |
| Ps05_409869744 | G/A | 95 | AGTAACGTCATTCGTCCAAAACC | chr5LG3 | 331834371 | 89.300 | −0.031 |
| | | | CAATCCATAACCCCGTAGTTAGC | | | | |
| | | | CGAACTATGACTTGCTCTGATTC | | | | |
| | | | TCATTCCACATGAGATACGTAGG | | | | |
| | | | CATAAGAC[[G/A]]TGATGTCTTAG | | | | |
| | | | CGAGCATACATCCCCCCAACCC | | | | |
| | | | ATAAGTCAGCCGAGCTACGAAG | | | | |
| | | | ACTTTGATTCTCATATTCATATG | | | | |
| | | | AGATACGTATGCAGTGGATGCG | | | | |
| Ps05_456631333 | A/G | 96 | CACATGAGTTGGCCTATCAAGA | 5 | 413429268 | 90.125 | 0.054 |
| | | | YGCATCACAGTCAAATTAGGAA | | | | |
| | | | CCTCATTCCAATACTTCACTACA | | | | |
| | | | ATCATTGATGCCACTRTTGCGRR | | | | |
| | | | AGCATCTGCC[[A/G]]TCCGGTTTT | | | | |
| | | | CATCTCGAGGGAKATGATGAAA | | | | |
| | | | TTCAACCTTTGTAAAGAAAGTTG | | | | |
| | | | AAATCCTCCTCGCATAATCTCTA | | | | |
| | | | TATGGTATCAAACCGGGTTGATT | | | | |
| Ps05_500234888 | T/G | 97 | AAATCAAGTGAARCAATTTTTTG | scaffold02833 | 50774 | 96.000 | −0.090 |
| | | | AACACTTAGTGTGAGATTAATA | | | | |
| | | | ATGTTTGGGATTCTATGTGGTAT | | | | |
| | | | TGTTGTTCTAAAAATCCAATCAC | | | | |
| | | | ARCCAAATG[[T/G]]TTTATGATCT | | | | |
| | | | ACACAACAAYGCTAATTTCAAA | | | | |
| | | | ATGTCTTTATAATTATGATCCAA | | | | |
| | | | ACAAAATTAATCAAACAATCAA | | | | |
| | | | TGCAATTGACAATTTGCAAACCA | | | | |
| Ps05_534247077 | T/C | 98 | CCAGTAACTRGTATATAAATYCC | chr5LG3 | 492763269 | 105.844 | 0.035 |
| | | | ACTTCTCCCCTCARAGTTAATMT | | | | |
| | | | TTGATATGTTCATCCTAACCGWT | | | | |
| | | | GACGRATTTTCTCCCTTTATGGT | | | | |
| | | | TTTCTACC[[T/C]]ARTAAAAAGGT | | | | |
| | | | AATTATAAATCCTGTTTTCCCCT | | | | |
| | | | GCTGAGTCTATCCTTTATATGTT | | | | |
| | | | CATCCTAACTGATGACAAATATT | | | | |
| | | | CTCTTTTTGGTATTCTATCC | | | | |
| Ps05_543517276 | A/G | 99 | GTCCAGGCCATCTGATCCTTGGT | chr5LG3 | 499899891 | 107.727 | 0.038 |
| | | | CCACATTTTAATTTGAGTCATAC | | | | |
| | | | AATGCAATTACCATGTGTGCAA | | | | |
| | | | GCGCGTTGACTGATGACCACTGT | | | | |
| | | | GGATGATAC[[A/G]]CGCTTGGCCA | | | | |
| | | | TCATATTTGCCACCTCAATTAAK | | | | |
| | | | GAGGGAGATCTTATGGCCCTCAT | | | | |
| | | | TTTTTTGAATTGACCGACTTTCT | | | | |
| | | | ATTTTAATATTTTAATTAGCA | | | | |
| Ps05_550603121 | A/T | 100 | TCATCCTACAACAAGAAATCAG | chr5LG3 | 143390359 | 109.621 | 0.044 |
| | | | ATGGGAGCAATCAATTCAATTAT | | | | |
| | | | YATYRTTAGAAGCAAAGTATTG | | | | |
| | | | TGACCTCGAAGACATAGGGATA | | | | |
| | | | ATAACATGTGT[[A/T]]RGGTTCAA | | | | |
| | | | TAGGGACATGAATAAATATGWT | | | | |
| | | | TTTCATTGCCATTTTTWGATTTT | | | | |
| | | | CGGTAATTTCCCCTTACGGGAAT | | | | |
| | | | TACTTCTTTATGTAATATCGCAC | | | | |
| | | | A | | | | |
| Ps05_551582581 | G/A | 101 | TATAATCATGTACTCCAWGAAC | chr5LG3 | 509926370 | 110.000 | 1−0.037 |
| | | | ACTCCAGTCRTAATAKACACACC | | | | |
| | | | AAATGACATCACTAAATACTCAT | | | | |
| | | | AGTGACCATATCTCATTCTGAAT | | | | |
| | | | GTAGTCTTC[[G/A]]RAATATCGTC | | | | |
| | | | TGACTTCACATGAATCTGATGAT | | | | |
| | | | AACCAAAACACAAATCAATCTT | | | | |
| | | | GTTAAACACACAAGCMCCAACC | | | | |
| | | | AATTGATCCATCAGATYATYGAT | | | | |
| Ps05_556990553 | C/T | 102 | CATAGAGATGATATGTTGGCTCA | 5 | 509729669 | 111.806 | −0.067 |
| | | | TAAGGGTATTCAATCAATAAAG | | | | |
| | | | ACAACCAATGCAAATAGGGATS | | | | |
| | | | ATTGGGTGACTGTTAAAATAGTT | | | | |
| | | | GGGACTCTTT[[C/T]]TGTCATAAA | | | | |
| | | | CACCATCGAAGTCRTAACTCAG | | | | |
| | | | GCCAACATCATAATAGGAAATA | | | | |
| | | | CATCRCATTGAGTGATTCTTCTA | | | | |
| | | | GTAGGTCCAGATGARAGGGTGT | | | | |
| | | | GT | | | | |
| Ps05_564305756 | A/C | 103 | TYRGCTTACATAKCCGAAAACA | chr5LG3 | 522716439 | 114.464 | −0.029 |
| | | | AGTTAAAARTGGCCTTAGAGGC | | | | |
| | | | CRAAGTAAARTACATGGTTGTCG | | | | |
| | | | AAGCCAAAGAGACTAAAGTACA | | | | |
| | | | CCTTGAAGGGT[[A/C]]TGGTGTGG | | | | |
| | | | ATTWTGAACCAAAGCCCCCTGA | | | | |
| | | | TAAAAAMYATGTCGAGCTGACT | | | | |
| | | | AGCTGTTCCTTAGCTTCGGTGAA | | | | |
| | | | TCCACACTCTCTCTCTCTCTCTA | | | | |
| | | | GY | | | | |
| Ps05_568744565 | T/C | 104 | GCTAAGTCAKGTATTCCAGTCGA | chr5LG3 | 124873928 | 115.700 | −0.062 |
| | | | TGCWAGGAATCRTCACTRAAAT | | | | |
| | | | TAGTTRTGTTTTCCCCCACAAAG | | | | |
| | | | TCRGGTATTCTAGTTGTCGCTAA | | | | |
| | | | GAATCATCA[[T/C]]TGAATTCACT | | | | |
| | | | TGGTCAATCCTCARTAGATATCA | | | | |
| | | | AGTATTCCAGCTATTGCTWGGA | | | | |
| | | | ATCGTCACTGAATCTATATTTTC | | | | |
| | | | CCAYCAGATTTAGGYRTTCCAG | | | | |
| Ps05_576520275 | A/G | 105 | TCCAAAACAGGTGGTTTGTTGAT | chr5LG3 | 535824046 | 117.417 | 0.032 |
| | | | TGATCCTCCATATTYAATTGTCT | | | | |
| | | | TTATTTGAGCATAATTTATCTTC | | | | |
| | | | CCTAGAGCTCACCCAATAGGCT | | | | |
| | | | AGGGTGTCT[[A/G]]CTCTGATGCC | | | | |
| | | | AACTGAAATTATGTTCCTCAGGG | | | | |
| | | | GATAGATGTCGAACATGATACT | | | | |
| | | | CCGACAAAAGGTTCKACAATAT | | | | |
| | | | ACATCAKAACCACTATAATAAC | | | | |
| | | | A | | | | |
| Ps05_591946858 | G/A | 106 | AAAAKGTCARACCAAGTTTAWA | chr5LG3 | 551226342 | 124.000 | −0.035 |
| | | | ATAAAGTCTTTGACAAGTAATA | | | | |
| | | | AGACATGTCCARGCTTTATATTT | | | | |
| | | | TTAATTAGGCATGGCCTATTTAA | | | | |
| | | | GTAAAGCTCG[[G/A]]CTCRGTCCG | | | | |
| | | | ACCTATTTCASCCCTACTTYTTCC | | | | |
| | | | CTTCACAAATTACAACAAACCTT | | | | |
| | | | AAGTTTCACTATTCACTTTCTTT | | | | |
| | | | ATTCTTRCCGYCTCCATCGTT | | | | |
| Ps05_596172019 | A/G | 107 | TGCTCTGGAAACTTTATCTTGYA | 5 | 547326524 | 126.100 | −0.037 |
| | | | TGATTGTTGTGAACCAATGGAAT | | | | |
| | | | GATGTACCCATAATAAATGTTAT | | | | |
| | | | GCGCCTTGACAGACCTGCTCATG | | | | |
| | | | TGTTTGCT[[A/G]]CAAAAGGGGTC | | | | |
| | | | ACTGATGACAAACCATGGTATC | | | | |
| | | | ATGATATTAAATGCTTTATCCAA | | | | |
| | | | AGACAAGAGTACCCACCTGGGG | | | | |
| | | | CATCAAACAAAGATAAGAAGAC | | | | |
| Ps06_1859845 | T/C | 108 | TAACCYTACTCTGATAGTGGTAA | chr6LG2 | 1621846 | 3.975 | −0.034 |
| | | | GTCTAACCACSAGAGAKAATATT | | | | |
| | | | TCATTGAAATYAATTCCTTTCTT | | | | |
| | | | TTGAGCATATCCTTTAACAACCA | | | | |
| | | | YTCTTGCA[[T/C]]GATATCTTTCCA | | | | |
| | | | CTTGATCATTACTATCACRCTAA | | | | |
| | | | TCTTATAGACACATTTGTTGYYG | | | | |
| | | | ATGGCTTTCCGACCYTCTGGAAG | | | | |
| | | | TACAAYAAGATCCYATGTC | | | | |
| Ps06_32259152 | A/G | 109 | GTATTTTTAAAGGAGCTCGCATT | scaffold00839 | 4002 | 18.826 | −0.050 |
| | | | AGCAAACTACCTCATTCACCATC | | | | |
| | | | TCCACCAAAAAGTTCATTCATTG | | | | |
| | | | ACGAGATATCGGTTTCTATGCAC | | | | |
| | | | ATATATAT[[A/G]]TCGAACAGATC | | | | |
| | | | ATCAATTTTGAGGGCRAYGATA | | | | |
| | | | ATTGCGRTTTTCGARCGATTTCG | | | | |
| | | | RCTTTGYTCGRTAAAGGAGACA | | | | |
| | | | CCATCAACTTATCCATGAGTTG | | | | |
| Ps06_71058460 | A/C | 110 | AAAGAAAAAGGGGTGARAATTC | scaffold05469 | 17855 | 32.400 | 0.053 |
| | | | ACATCAATAACGACATAARMTG | | | | |
| | | | GAAGGTAGARTTCAAACATCTA | | | | |
| | | | TACATTCARTAAYAATCACACA | | | | |
| | | | ACATCATTATCA[[A/C]]ACCCAAT | | | | |
| | | | CCATCAACATACATAAAACAAT | | | | |
| | | | CATATATTCATCATTTAAAWTTA | | | | |
| | | | TGCAATGRGMCTCACAACCTCA | | | | |
| | | | ACTAGACTCATATGCACGTGGTA | | | | |
| | | | CCT | | | | |
| Ps06_75832558 | C/A | 111 | ATAGTGCAAAACATGCCTTTGA | chr6LG2 | 62010766 | 33.900 | 0.049 |
| | | | GAATTTTGCCAAATTGGTCCAAC | | | | |
| | | | TTCAAGCCCTTCTGTTTTRATGA | | | | |
| | | | TGCAAGCCCYAAATGGGAAAAC | | | | |
| | | | CTCCAACRTC[[C/A]]AAGTTGTAT | | | | |
| | | | ATCTTTTCAATACCATCAAAATG | | | | |
| | | | GACTTAAATTTTGCATCATTTGG | | | | |
| | | | ATTTTTTATGAAGGAGTTATGGG | | | | |
| | | | CACTTGAAGTTGGACTTTTTTG | | | | |
| Ps06_79052113 | A/T | 112 | GCATGATGAACTAATAAAAATA | chr6LG2 | 64688438 | 34.346 | 0.041 |
| | | | TRYCTAAGCTATTATATGWTCAT | | | | |
| | | | GGGGTGGGGTGGGGGGGGGCAT | | | | |
| | | | TTGACCCACAATGGGGGGTGTA | | | | |
| | | | AGCAATACAAC[[A/T]]TAAATGGA | | | | |
| | | | AATGGAAAGAAWTATACAAGTT | | | | |
| | | | ACAAACTATGTATAAYGYCTAA | | | | |
| | | | AACATACAAGTGRCATGAYACT | | | | |
| | | | TCAAACAATACAAGTCGTGAAT | | | | |
| | | | GAGT | | | | |
| Ps06_91302660 | T/C | 113 | AAAATMTCTCTCTCTCTCTATTC | chr6LG2 | 75309486 | 37.846 | 0.027 |
| | | | CCTCTCAATTTTCACTCTCCCTAT | | | | |
| | | | CATTTCATTCAATTCACAAACTC | | | | |
| | | | GACCCTCATATTTCAGCTCTTTC | | | | |
| | | | ATATTCA[[T/C]]ATGTTTACTCTCT | | | | |
| | | | CTGCTAATTCAATCCAATTCAAT | | | | |
| | | | CCTCAAGATCACTCTCTCAGCRT | | | | |
| | | | GAAACTCTCAGAAGAAGAAGTC | | | | |
| | | | TCAGAKATAGGAAGAAAAA | | | | |
| Ps06_97595572 | C/T | 114 | TCATGTAGGACTGMACATGAAA | chr6LG2 | 86301013 | 39.000 | 0.044 |
| | | | GATTTGAAGTCGTRCAAAGGTG | | | | |
| | | | GGAACTTTTTGGAATTCAATGAC | | | | |
| | | | TCTTCGACTCGTCCATGTGRAAC | | | | |
| | | | CATARACCAC[[C/T]]AGTCTCGYT | | | | |
| | | | TGAAGAAGGTAAAAATAAGATA | | | | |
| | | | ACTATGAAAGTGTTTTTCCTCAT | | | | |
| | | | CATCCCTTGTGAAAKCATCTACA | | | | |
| | | | ATGACATCTTAGGAAGGTCATTT | | | | |
| Ps06_108179595 | G/A | 115 | TTGTGGCTGGAATTGACAATCCA | chr6LG2 | 89382481 | 40.998 | 0.040 |
| | | | GYRGTGAAAAGGAAGAATTTTC | | | | |
| | | | TTTTCTCCAAAATTGCTTYGGTA | | | | |
| | | | RAACTYTGTGGAAGTTGCTACAT | | | | |
| | | | ATGGAGYTG[[G/A]]AGTTTCTATT | | | | |
| | | | GCCTTCAAACATATCAAGGCTCA | | | | |
| | | | AATAAGGAACTAGTGGGACTTA | | | | |
| | | | GGATAAGTTGTCGTGAAACGGA | | | | |
| | | | CGCTTGGAGATAGTTAATTGATT | | | | |
| Ps06_137271101 | C/T | 116 | CTYGCAATAAGGTAGCTACAAT | chr6LG2 | 111705293 | 45.500 | 0.033 |
| | | | CAAAGGTTTGTTCGGAGCGTTTG | | | | |
| | | | AAGCAYTTGGTTCAACTCGAAC | | | | |
| | | | CAAGGGGAGAGAAGTAAATAKG | | | | |
| | | | ATCTACAAMAA[[C/T]]ACTTAGGT | | | | |
| | | | TTGMTTGGTATTGATCATTTCTT | | | | |
| | | | CTCTTGTATAAACTTTGCAAWTG | | | | |
| | | | ATAATAAATATCTCAATTCTAGT | | | | |
| | | | TTTARAATTRRGGGCAGACGTAC | | | | |
| Ps06_261243645 | G/A | 117 | AATGTAATTTAGTTGAAGTAGA | chr6LG2 | 191916418 | 54.900 | 0.050 |
| | | | AATGACTGGTAAATAATTCACTT | | | | |
| | | | GGAGTARGCCTTGTACTAGTAAT | | | | |
| | | | CTAATTGTCTATCGCAAGTTGGA | | | | |
| | | | TAGAGCACT[[G/A]]GTTAACGTTG | | | | |
| | | | CWTGGYGCATGGCTTTCCCTGG | | | | |
| | | | TGCATATTTTCRGGTGTTGTGTA | | | | |
| | | | AWTTTCATTCTAATCACAACCTC | | | | |
| | | | ATTCTCCTTAGATGTGGCCTTM | | | | |
| Ps06_375201129 | T/C | 118 | ATAGTTAATTAATAGCTAATTAA | chr6LG2 | 374758162 | 84.486 | −0.029 |
| | | | CAATTTTTTTCATATCACATCCT | | | | |
| | | | AATTAAAACTAGTCTTGAGATG | | | | |
| | | | GTTGTGTAGGTTGGGGTGCCTGT | | | | |
| | | | GATGARCTT[[T/C]]CAATCTCTATT | | | | |
| | | | GCACATGTCTCTKTAGTGTTTGT | | | | |
| | | | AGTCTTTGTCTTTTTTCTTTTTTT | | | | |
| | | | TTCTTCATAATTTGTAGTGTTTGT | | | | |
| | | | AGTATTTTCCTTCTTATT | | | | |
| Ps06_383667570 | T/G | 119 | AGCCAAGGGTCTGATGCTTGTCT | chr6LG2 | 303558968 | 87.000 | 0.100 |
| | | | CYGCACTAAAAWTAACSCCAAC | | | | |
| | | | AAATCAAATCATCTTTTAYCTCA | | | | |
| | | | GTGYACTCTTCAAAYMTTTMAA | | | | |
| | | | AAAGRACGTG[[T/G]]TACTTCCRC | | | | |
| | | | TCTACCGTGAACGRCGCTTAAGC | | | | |
| | | | CTCCATGTGTGAGCAASTAATGT | | | | |
| | | | TTAACTGCTAGAGTGTGATCCAA | | | | |
| | | | GCAGATTGATAAACAACCAAAC | | | | |
| Ps06_402503684 | T/C | 120 | AAAATGGTACAAATAAAAATTT | chr6LG2 | 401325650 | 92.143 | −0.046 |
| | | | ATTAAGAAAAAAAATAACAACC | | | | |
| | | | AACAAATCAACAAAACATACAA | | | | |
| | | | AGTAGATCACAAAAAACACCCA | | | | |
| | | | AGCATATAAAAA[[T/C]]CAATCTC | | | | |
| | | | GTAGAATTTAGGTTAGAGCTCAT | | | | |
| | | | ACTRCCATAACCACCATAAATA | | | | |
| | | | GATCATAATTCACAAAGAGAGT | | | | |
| | | | AACGAAAYCACAAACCAAATCC | | | | |
| | | | TATC | | | | |
| Ps06_410567663 | C/T | 121 | MCCTTTTGGRTGTTCAAATACAC | chr6LG2 | 406586297 | 94.016 | 0.031 |
| | | | ATMTTYATTTATAGCCATTTAGG | | | | |
| | | | AAGGCACTTTTAACAACCATTTG | | | | |
| | | | GAATAGCTYGAWCTTTAAGAGA | | | | |
| | | | CATGYCATT[[C/T]]CAWGTAACAA | | | | |
| | | | TCTGATAGACTCCAGGMGAGCT | | | | |
| | | | ACTRGAGMAAAGGTTTCATCAA | | | | |
| | | | AGTCTACTCCTTCCAYTTGGGTG | | | | |
| | | | TATCCTTGAGCAACAAGTCTGRC | | | | |
| Ps06_427519500 | C/G | 122 | ATCAAAAAACATAAATGAAGAT | chr6LG2 | 426328393 | 98.981 | −0.048 |
| | | | GATGTCGATTCAACGCAGTATA | | | | |
| | | | GAAGACTCATTGGATCACTTTGA | | | | |
| | | | TATGTCACACAAGGCCTAATATA | | | | |
| | | | RCATAGTGTA[[C/G]]GTATGGTGA | | | | |
| | | | GAAGATTCATGCAGAAGCTAAA | | | | |
| | | | TGTATCACACCTAGCAACTACGG | | | | |
| | | | AGAGGATACTAAGGCATCTGAA | | | | |
| | | | AGGAACTCTTGACAATGAAATTT | | | | |
| | | | A | | | | |
| Ps06_446483044 | G/C | 123 | AATCTTGCACTTTTCGACCAGTG | 6 | 438943398 | 104.563 | −0.025 |
| | | | AAAACTGACCCCTGGTCGGTTGT | | | | |
| | | | AATAGTTTATGGGATGCCAAAC | | | | |
| | | | CTGCAGATGATGTAACTCAGGA | | | | |
| | | | CAAAGTCTAT[[G/C]]ACAGCCTCT | | | | |
| | | | TGGTCCACATTTGTTAATGCTAC | | | | |
| | | | GGCTTCGACCCATTTTGTGAAAT | | | | |
| | | | AATCGATACCGACTAACACATA | | | | |
| | | | CCTTTGTTGTTTCGACGAGGCTG | | | | |
| Ps07_9801763 | G/A | 124 | GCCARCAYTTCTGAAACCAAGTT | chr7LG7 | 8316015 | 2.437 | −0.041 |
| | | | MTTATGGTTTGAGCTTTCATATG | | | | |
| | | | ATGCAAGTATTTGGATCCACTTG | | | | |
| | | | AGTTCATYTGCTACATGGTCTGG | | | | |
| | | | ATGCAACT[[G/A]]TAACTTTGAAC | | | | |
| | | | CTGGGTCTAATGCCTTATTCAGG | | | | |
| | | | GYCAATCAAGAARTATGTCATCT | | | | |
| | | | GATACTTGGGAAGATCAAGAAG | | | | |
| | | | AAGATTGTGAAGTTGCTTGCT | | | | |
| Ps07_20773355 | A/G | 125 | ATGGCCTTAGGTTTATATGTTAT | chr7LG7 | 19382049 | 7.295 | −0.038 |
| | | | GGTGTAGCGCTTCAACCTAGGGT | | | | |
| | | | GCTCGAGTGTACTCAAATATCGT | | | | |
| | | | TATGTTTTTCGAGGTATCACTTG | | | | |
| | | | GACTGTTG[[A/G]]TTTTTTACCTTC | | | | |
| | | | CATTTGCTKTCATTCTACAACAT | | | | |
| | | | TGATTTGTTTCATCTAGTCCATT | | | | |
| | | | ATATAAGGGTTGCTCGGACTTTT | | | | |
| | | | CGAACAATTTTGTGTTCCC | | | | |
| Ps07_46743665 | G/T | 126 | CCTAAACCTCTTGTCAAACAAAT | scaffold06512 | 13304 | 16.686 | −0.028 |
| | | | GCATCAAAAGGGTATCAAAACT | | | | |
| | | | CAAAGTCAGSGGGCAAGGTTCC | | | | |
| | | | ACACATAAAGACATGACATACA | | | | |
| | | | TACTAAAACAT[[G/T]]TGCAAAAG | | | | |
| | | | GATTGGTTTCAACATTCAAAGGA | | | | |
| | | | AGTGACACAAGTGCCTCATGCT | | | | |
| | | | AAGCAAGCATCAAAATAAGGAC | | | | |
| | | | TAAAATGCAAACATCAACTTCA | | | | |
| | | | AAA | | | | |
| Ps07_50335973 | T/C | 127 | ATACAACAACAASTAACATTAAT | 7 | 52311972 | 17.332 | −0.064 |
| | | | AAGTTACTTTMATGATTTYYTAA | | | | |
| | | | CAAACATTAAACATTTTTAACAA | | | | |
| | | | GGAATTACTAGAGAAAGATTAC | | | | |
| | | | ACTAAGAAA[[T/C]]CAACCTTGAA | | | | |
| | | | AYATTTTCATGCYCTTKCTTTCA | | | | |
| | | | TTACCATTGTATTCATTATGTTA | | | | |
| | | | AAGTTTATATTTCACCACTTTGC | | | | |
| | | | ATCCAATRTGGATAATTRTGG | | | | |
| Ps07_55350864 | G/A | 128 | AGCATCAATCTGCTTCTGCACTT | chr7LG7 | 50802012 | 17.986 | −0.032 |
| | | | CCTCTTTGATCTTCACTGCCATA | | | | |
| | | | TCAGGATGAGTTCTTCTCAATTT | | | | |
| | | | CTGCTTGACTGGCGGGCATTCTC | | | | |
| | | | ACTTCAAC[[G/A]]GCAATYTATGC | | | | |
| | | | TCCACAATCTCAAAATCCAAACC | | | | |
| | | | AGACATGTCTTGATAGGACCAA | | | | |
| | | | GCAAACACATCAGAATACTCTC | | | | |
| | | | GAAGAAGATCAATCAACCCATT | | | | |
| Ps07_57031312 | G/A | 129 | AGTTAATTGGATTAAAAGAGTTT | chr7LG7 | 52153701 | 18.200 | 0.049 |
| | | | ATTATTGAAAATTTCYGGKTTTC | | | | |
| | | | TTTTTGGCATTTTGATTCTCAAT | | | | |
| | | | AYTAATCRGGGMAAACCTCCAC | | | | |
| | | | TTCGTCGGT[[G/A]]ATCSTTCACCT | | | | |
| | | | CTTCCACACTCTTGCCATGAATT | | | | |
| | | | ATGGATGTTTGCGTGTGATTAAA | | | | |
| | | | TTAAGCCACTGTTGTGATTTGCT | | | | |
| | | | TATTAGAATATTCACTTATT | | | | |
| Ps07_58281807 | A/G | 130 | ATAACACTCGTGTACCAATGYA | chr7LG7 | 56383957 | 19.079 | −0.026 |
| | | | CATACCAAATAGAARAAAAGTT | | | | |
| | | | TAAAGAAAAAAAGTARGATTTG | | | | |
| | | | RTACATACCATACAAGTTTCACA | | | | |
| | | | CACATACCGGT[[A/G]]GCARGTAC | | | | |
| | | | ATACCCGATACTGATACTTTGTC | | | | |
| | | | TAAAATGACCTATCAATGYTTCA | | | | |
| | | | CATRTTCTCACCCATTATGCATG | | | | |
| | | | TTTCAAACTTCCACGATAAATTT | | | | |
| Ps07_84885129 | G/A | 131 | AGTTGGAGTCTAAGGAAAAAGA | chr7LG7 | 1311773 | 26.594 | −0.030 |
| | | | TGCACTTATTAAGCTACTTGAAG | | | | |
| | | | ACCGAGTGASAAAGAGATAGAG | | | | |
| | | | AGAGCCARAGGTKTCATCCTCTA | | | | |
| | | | GCATGCCTYA[[G/A]]CCTTCCGCT | | | | |
| | | | GCTTGGAAGAAGATTGYTGATC | | | | |
| | | | AGCTTGTCCTCGAGAAGACTCA | | | | |
| | | | GATGAAGGCTTCTTTTGAGACCG | | | | |
| | | | AGATYCGTCGCATTCGAAGGAA | | | | |
| | | | GT | | | | |
| Ps07_89781713 | T/G | 132 | ARTTTCTGAATAATMATTTGRTT | chr7LG7 | 88161594 | 27.805 | 0.033 |
| | | | AGYTGTAMTTTAAGCTGGATTA | | | | |
| | | | TTYGTAATGATATCCTCTTTAAT | | | | |
| | | | GGYGGGATGAGAGGGTTCCMRA | | | | |
| | | | AATTGTYTTG[[T/G]]TGGCTAAAT | | | | |
| | | | TGRTTTTTTGGGATTGGTTTRGA | | | | |
| | | | GTTAGATTTAAGGACATTCTTCC | | | | |
| | | | ATATSTTAGGTTTTRTATTTGAGT | | | | |
| | | | TAAATATCCTYTTAMTACRAT | | | | |
| Ps07_112377551 | G/A | 133 | AGCACTCTTACCGAACAGATCCT | chr7LG7 | 45871271 | 35.548 | 0.038 |
| | | | TTCCCCTCAAGGTTTTCAACTCC | | | | |
| | | | TTTCGCAGCTCAAGGAATTGGTC | | | | |
| | | | TTTCATTTCATCCATCTTCTCATA | | | | |
| | | | AACATCC[[G/A]]GACCTTCAGATG | | | | |
| | | | GCTCGAAATGATAGATGGTGTC | | | | |
| | | | CTCAACGCGGGGCAAAGTATGA | | | | |
| | | | ATAACTGTTGGAGGCACAGACA | | | | |
| | | | TGATYGGGCTAGATGTCGGCAT | | | | |
| Ps07_131261098 | T/C | 134 | WTGAACCTCGTTGGAAGCCCAA | scaffold02959 | 72341 | 39.608 | −0.049 |
| | | | ACTGGCTCTKTTGTTGTTGTCGG | | | | |
| | | | ASACCTCTATCATTTGGCCCCAC | | | | |
| | | | TGATCAGAATTACCATCTTCAAC | | | | |
| | | | AATCTTYTG[[T/C]]GCATCTTTAA | | | | |
| | | | ATRAGGACATGGSTKCCCCAATT | | | | |
| | | | CTCTTTTCCTTAGCAATAGACAA | | | | |
| | | | GGCTTGGAACGGAGTTCCAATCT | | | | |
| | | | CATCTTYAGGTTCTACATAYG | | | | |
| Ps07_155895151 | G/A | 135 | GGTTYTGAGATGRAACACCTTCC | chr7LG7 | 89032441 | 46.200 | −0.037 |
| | | | CTATTTATAGGGAAGGTGTTGGG | | | | |
| | | | ATGGATARGKGATAGTAAGGGC | | | | |
| | | | GAGGTTTGATTAGAGATTGCAA | | | | |
| | | | GTGACTTGAA[[G/A]]CATGCTCAA | | | | |
| | | | AAGTKGGATWTTTGGGATAAGG | | | | |
| | | | ATTGGCGTKTTCTGACYTGGTTT | | | | |
| | | | GCATCACCAACCAACTGGTTTTT | | | | |
| | | | CWCCTTAGCATCAAACAAGTGC | | | | |
| | | | A | | | | |
| Ps07_173321635 | G/A | 136 | GTCACTTCGATAGCTTTGTCRCA | chr7LG7 | 16557044 | 50.158 | 0.039 |
| | | | CAAGGTCTAACTTAGCATACAGT | | | | |
| | | | GTAGGTAKGATGAGTAGATTCA | | | | |
| | | | TGCAAAAGCCAAAGGTATCACA | | | | |
| | | | TCTAGCAGCG[[G/A]]TGAAGAGGA | | | | |
| | | | TACTAAGGTATCTAAAAGGAAC | | | | |
| | | | TCACRACTATGGCATTTTGTTTC | | | | |
| | | | CTGCAGCTGATGAAGGAAAATA | | | | |
| | | | ATRCAAATTAGTGGGATACACC | | | | |
| | | | GA | | | | |
| Ps07_231299734 | C/T | 137 | CAATGAACACCMAAACATCTCA | Ichr7LG7 | 223304507 | 61.215 | 0.051 |
| | | | TCACCAYAACACTCAATCATTGT | | | | |
| | | | TTGACTTTAGTACACCACRAGAA | | | | |
| | | | CCATTACTTTGTTTCCAAAACGA | | | | |
| | | | TTCAATGTC[[C/T]]CAATTCGGGC | | | | |
| | | | AACCATACCGTCCATAATTCACC | | | | |
| | | | CAACCACAACGACCCAACTACG | | | | |
| | | | ACAACATGGACATCGAACTCAA | | | | |
| | | | TTACGGRAGCACCGTTGGCGAC | | | | |
| | | | A | | | | |
| Ps07_235684752 | C/T | 138 | AATTAAAAAGAAAAATAGAAAA | chr7LG7 | 310437720 | 62.192 | −0.052 |
| | | | AATGAGGGCCATCAGATCYCCC | | | | |
| | | | TYATTAAYTGAGGTGGCAGATC | | | | |
| | | | TGATGGCCAAGCGCGYSCTATCT | | | | |
| | | | ACCATGGTCCT[[C/T]]AATCAATG | | | | |
| | | | CGTCACAKGAGTGRTAATCAGA | | | | |
| | | | AGGAGCGGMTCAGATTAAAAYA | | | | |
| | | | TTTCAAACAAATCAAATGGCCA | | | | |
| | | | AGAGACGCGCCAACGCATCAMC | | | | |
| | | | GRAG | | | | |
| Ps07_238767894 | T/G | 139 | TGCAYATATATATACMTTGTTCG | chr7LG7 | 310515874 | 63.928 | −0.029 |
| | | | AGCTTCTCCAAGTCTTCCAAGTC | | | | |
| | | | CTTTTCAWTATCCACTTTCAAAG | | | | |
| | | | GGGGAGATCTTAAMCAATTTTG | | | | |
| | | | TGCACATAT[[T/G]]AAAGCTTCAA | | | | |
| | | | CAATGTATGGTGTAAGAGAAAT | | | | |
| | | | CCTATAACAACTAAGAACTCTAC | | | | |
| | | | CCCCTGTGCTAAAAGCAGACTCT | | | | |
| | | | RAAGYAACAGTAGATATTGGCA | | | | |
| Ps07_241735133 | G/A | 140 | ATCTGAATTTACTAGGGKTACTC | chr7LG7 | 158981077 | 64.370 | 0.045 |
| | | | CTAATGGTGATGTTAATCATGAT | | | | |
| | | | CAAAKTACATCTTAAAAGAATA | | | | |
| | | | ATTGTATTGCTAAACCTCCAACA | | | | |
| | | | TTCAGTGGA[[G/A]]AATCTACTGA | | | | |
| | | | ATTTGAATGGTGGAAKAGCAAG | | | | |
| | | | ATGTACACTAAAATCATAGGTCT | | | | |
| | | | TGATGATGGGTTATGRGATATGT | | | | |
| | | | TAGAAGATGGMATTTACATCAA | | | | |
| Ps07_274069066 | C/G | 141 | CTTRATCTTGAGWTKCTTTRRTT | chr7LG7 | 335690162 | 72.370 | −0.038 |
| | | | ATCATCAATGTTGAAGGCKATTT | | | | |
| | | | TCATGYGTTRTCGTGATCAAAAC | | | | |
| | | | TCCAGATGTCGATCTTCATTACT | | | | |
| | | | TSCAAGCA[[C/G]]ATAGATTCACA | | | | |
| | | | TACTGCCCYGGCACCCCYAAGT | | | | |
| | | | CRCCTACCTACAACAAACAGKT | | | | |
| | | | GGGCCTATAACATGTTTRAAATT | | | | |
| | | | TTMACTTATTGTGTKTCTCTTA | | | | |
| Ps07_314769485 | JA/G | 142 | CCTAAATCTCCCACCTYCCGCCA | 7 | 322450055 | 80.500 | −0.040 |
| | | | YAYGAGCCTCTSGTTAACCATTG | | | | |
| | | | TATAAATCATCACCATRATGTCS | | | | |
| | | | CTTCTCTCATGCATATCCTTCAT | | | | |
| | | | CCAGCATA[[A/G]]CATGAAGCCTT | | | | |
| | | | GAAAATAAACAAGCAAAGCTAA | | | | |
| | | | AGTCCAATTCTTATTGKCACATT | | | | |
| | | | GRAGGCCCTACACAAAAAGAGT | | | | |
| | | | ATCACCYTTGTAAATACCATAA | | | | |
| Ps07_327087818 | G/A | 143 | CTCAGATGGTTGGTGCTGACAAC | scaffold00840 | 10989 | 81.186 | −0.028 |
| | | | CATGAGAYCAACYAGGGACAW | | | | |
| | | | RCTCTTYAKACTTCTRTGYCAGT | | | | |
| | | | TACTATTGAAACTCCTGATGATA | | | | |
| | | | ACAAAGATKA[[G/A]]TACAAAGG | | | | |
| | | | TCCTCACCTTCATTTTCATGTTCC | | | | |
| | | | TCCTCAGTCTGCTAAAGCTACTA | | | | |
| | | | TTCAAAATTTGAATCAAGGTGTT | | | | |
| | | | CAGATACCTCCTCCTTATTCTR | | | | |
| Ps07_337883272 | T/C | 144 | TTAYGAGGATCTTTGGTGGAGTT | chr7LG7 | 365136400 | 82.190 | 0.032 |
| | | | AAGGGAGTTAGGGCCACCCCAA | | | | |
| | | | GCGGTCCTTCCTCCCGTTTCTGA | | | | |
| | | | TGTCAACGSTTGGTGCGAGTTCC | | | | |
| | | | ATTYTGGCG[[T/C]]TCCCGGTCAT | | | | |
| | | | TCAATCGAGAACTGTAAATCCTT | | | | |
| | | | AATGTATAAAATGCAAGATTCG | | | | |
| | | | ATAGATTCTAAGGCATTGCGTTT | | | | |
| | | | GCGCCTAACATCCCAAATGTAA | | | | |
| Ps07_466233654 | A/G | 145 | GAATTTGTTGACCTTYTGTGATG | chr7LG7 | 460750292 | 101.754 | −0.050 |
| | | | AACACATCCCAGAAGAATTCTA | | | | |
| | | | GCCCAAACCTTTAGCTTAGGGTG | | | | |
| | | | AAAATCCTTGACCTTRCTAGAGT | | | | |
| | | | GTTTTCGAG[[A/G]]GACGAAARTC | | | | |
| | | | ACTTCGGAAATCTTTGTCAAATT | | | | |
| | | | GGATTCCTTTTCCACTAGTTGTT | | | | |
| | | | CACATMTGATTCTAGACCCATC | | | | |
| | | | ATATCCAATGAGTTTGGCAATA | | | | |
| Ps07_466821729 | A/G | 146 | TTATTGTTGACCCATATGAYATC | scaffold01757 | 47207 | 102.930 | 0.059 |
| | | | TCTTGTATTYTATRTGGGTATTT | | | | |
| | | | YCTAGAATCAGTCTCTCATTTGT | | | | |
| | | | TTRTTACCTRTGTTTTAGCTTCGA | | | | |
| | | | AGATTTG[[A/G]]TATATGATTTTC | | | | |
| | | | AAGTGGTTGTGGTGGGTGGTGG | | | | |
| | | | TTATCCMTAGGGATCMTAGGAT | | | | |
| | | | TCTTTTTKAGATTTTACATCTTTA | | | | |
| | | | KGTGATRGGGMTAAGCTGAG | | | | |
| Ps07_482615897 | G/A | 147 | ATCGAGAAAAGTCCCAATAAAT | chr7LG7 | 481276628 | 113.999 | 0.086 |
| | | | ATAAAAATGYTCTCTCTTGYTGA | | | | |
| | | | ACYCCTAAATAAATGTCCCAAC | | | | |
| | | | AATGGTATCAAAGCCCRGTTCG | | | | |
| | | | ATTGAARGACY[[G/A]]RCTCACTT | | | | |
| | | | GTATYRAAAGTACTCCCCATAG | | | | |
| | | | GTGGTGGGTAGGAGGTGTCCCG | | | | |
| | | | TTGAAGAGTGTCAACGGTGGTA | | | | |
| | | | CAAGTGTATRTGGAAGAAAGAA | | | | |
| | | | CTTA | | | | |

### Example 2. Identifying the Top Genomic Regions (QTLs) Associated With High-Protein Phenotype in Pea Seeds

Based on the GWAS results described above, two high protein QTLs, chr3-4 and chr5-1, were identified after filtering for p-values, as provided in Table 2 below, as mapped to a public reference Pisum sativum genome, Cameor (Pisum sativum Cameor genome v1a).

TABLE 2
QTLs Associated with High-Protein Pea Phenotype
Mapped to Cameor Reference Genome

| | Chr3-4 | Chr5-1 |
|---|---|---|
| QTL | Ps03_531239107 | Ps05_49389403 |
| p-value Peak | 4.15 × 10⁻⁹ | 4.69 × 10⁻⁷ |
| Peak SNP Cameor | Chr5: 563531992 | Chr5: 36,835,261 |
| p-value Range | <1.00 × 10⁻⁶ | <1.00 × 10⁻⁵ |
| Peak Range Cameor | Chr3: 421,821,200-437,541,609 | Chr5: 36,153,174-38,589,532 |
| Size [bp] Cameor | 15,720,410 | 2,436,359 |
| Peak Cameor ID | Psat5g296280 | Psat5g018160 |
| Cameor IDs Range | Psat3g198400-Psat3g208320 | Psat5g018800-Psat5g020640 |
| Gene Model Count | 234 | 44 |
| Cameor | | |
| Alleles | T/C | A/G |
| High protein allele | C | A |
| Beta (effect size) | 4.49 × 10⁻¹ | 4.90 × 10⁻¹ |
| Genetic Map Position | chr3: 113.3 cM | chr5: 9.6 cM |
| Cameor | | |
| ~20 cM range | Chr3: 421,829,254-437,541,609 | Chr5: 1-54,716,217 |
| (+/−10 cM) Cameor | (end of chr) | |
| LD block [bp] | 1,200,000 | 10,600,000 |
| Pleiotropic effect | yield, lodging, height | yield, lodging, height |

### Example 3. The Chr3-4 and Chr5-1 QTLs are Associated With Increased Protein Content

Table 3 shows mean protein %, mean yield, mean lodging, and mean height of the pea plants having homozygous reference alleles, heterozygous alleles, and homozygous alternate alleles of the chr3-4 QTL. As shown in Table 3, the alternate allele of chr3-4 QTL is associated with increased protein content by 1.23% dry weight, which is a 4.6% increase in the protein content relative to the reference allele of chr3-4 QTL without significant decrease in yield.

Table 4 shows mean protein %, mean yield, mean lodging, and mean height of the pea plants having homozygous reference alleles, heterozygous alleles, and homozygous alternate alleles of the chr5-1 QTL. As shown in Table 4, the alternate allele of ch5-1 QTL is associated with increased protein content by 1.04% dry weight, which is a 3.0% increase in the protein content relative to the reference allele of ch5-1 QTL, with an increase in yield by 1.9 bushel/acre or 3.9%.

TABLE 3
Characteristics of Pea Plants Associated with Chr3-4 QTL

| | | Mean_Yield | Mean_Lodging | Mean_Height |
|---|---|---|---|---|
| Alleles | Mean_Protein % | (bushels/acre) | (1-9)* | (inches) |
| Homozygous | 26.50 | 50.00 | 3.37 | 25.72 |
| reference allele | | | | |
| Heterozygous | 27.06 | 48.77 | 3.47 | 26.41 |
| reference/alternate | | | | |
| Homozygous | 27.73 | 49.57 | 3.20 | 26.58 |
| alternate allele | | | | |
| Difference in | 1.23 | −0.42 | −0.17 | 0.86 |
| homozygous alleles | | | | |

*Lodging units from 1 to 9, with 9 being the worst lodging

TABLE 4
Characteristics of Pea Plants Associated with Chr5-1 QTL

| | | Mean_Yield | Mean_Lodging | Mean_Height |
|---|---|---|---|---|
| Alleles | Mean_Protein % | (bushels/acre) | (1-9)* | (inches) |
| Homozygous | 26.48 | 48.23 | 3.43 | 27.03 |
| reference allele | | | | |
| Heterozygous | 26.88 | 48.44 | 3.40 | 27.19 |
| reference/alternate | | | | |
| Homozygous | 27.52 | 50.12 | 3.23 | 25.97 |
| alternate allele | | | | |
| Difference in | 1.04 | 1.89 | −0.20 | −1.06 |
| homozygous alleles | | | | |

*Lodging units from 1 to 9, with 9 being the worst lodging

1. A method of producing a population of high-protein pea plants or seeds, said method comprising:
a) genotyping a first population of pea plants or seeds for the presence of at least one high-protein molecular marker that is within 20 centimorgans of one or more high-protein Quantitative Trait Locus (QTLs) selected from the group consisting of Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps02_432513197, Ps02_440456554, Ps03_158264810, Ps03_205819517, Ps03_206829164, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897;
b) selecting from the first population one or more pea plants or seeds comprising one or more high-protein alleles having the at least one high-protein molecular marker; and
c) producing a second population of progeny pea plants or seeds from the selected one or more pea plants or plants grown from the selected seeds, wherein the second population of progeny pea plants or seeds comprises the one or more high-protein alleles having the at least one high-protein molecular marker,
and wherein the second population of progeny pea plants or seeds are high-protein pea plants or seeds, thereby producing a population of high-protein pea plants or seeds.

2. The method of claim 1, wherein said at least one high protein molecular marker is within 10 centimorgans of said one or more high protein QTLs.

3. The method of claim 1, wherein the one or more high-protein molecular markers confer a yield penalty of less than 5% under normal growing conditions, or wherein the pea plants or seeds comprising said one or more high-protein alleles have yield that is 99% or greater relative to pea plants or seeds without said one or more high-protein alleles.

4. The method of claim 1, wherein genotyping comprises assaying a single nucleotide polymorphism (SNP) marker or a haplotype.

5. The method of claim 1, wherein genotyping comprises the use of an oligonucleotide probe or a pair of primers.

6.-7. (canceled)

8. The method of claim 1, wherein said one or more high protein QTLs are Ps03_531239107, Ps05_49389403, Ps03_531014546, Ps03_531232613, or Ps03_531239107.

9. (canceled)

10. The method of any one of claims 1-9, wherein pea plants or seeds comprising said one or more high-protein alleles have protein content that is greater by at least 1.0% dry weight relative to pea plants or seeds without said one or more high-protein alleles, or wherein the resulting population of high-protein pea plants or pea seeds comprises at least 20%, 21%, 22%, 23%, 24%, 25%, 26%, or 27% protein by weight.

11.-12. (canceled)

13. The method of claim 1, wherein the second population of progeny pea plants or seeds further comprise one or more alleles associated with high yield.

14. The method of claim 1, wherein the method further comprises determining the protein content of the second population of pea plants or seeds, wherein the second population of pea plants or seeds having the one or more high- protein alleles have an increased level of protein when compared to a control population of pea plants or seeds lacking the one or more high-protein alleles.

15. A high-protein population of pea plants produced by the method of claim 1, wherein said high-protein population of pea plants has a greater frequency of the at least one high-protein molecular marker than said first population of pea plants.

16. (canceled)

17. A method of introgressing a high-protein QTL, the method comprising:
(a) crossing a first pea plant comprising a high-protein QTL with a second pea plant of a different genotype to produce one or more progeny plants or seeds; and
(b) selecting a progeny plant or seed comprising one or more high-protein alleles of a polymorphic locus linked to the high-protein QTL,
wherein the polymorphic locus is a chromosomal segment comprising any marker within genomic regions of a Pisum sativum genome corresponding to genomic regions 421,829,254-437,541,609 of chromosome 3, 1-54716217 of chromosome 5, 20877277-371072249 of chromosome 1LG6, 10842575-426699364 of chromosome 2LG1, 104891818-425968089 of chromosome 3LG5, 5972665-445125850 of chromosome 4LG4, 362278-547326524 of chromosome 5LG3, 1621846-438943399 of chromosome 6LG2, 8316015-481276628 of chromosome 7LG7, scaffold 02116, scaffold 04655, scaffold 00066, scaffold 03789, scaffold 02021, scaffold 00644, scaffold 00254, scaffold 02127, scaffold 00706, super-scaffold 888, scaffold 00462, scaffold 02449, scaffold 02833, scaffold 00839, scaffold 05469,, scaffold 06512, scaffold 02959 scaffold 00840, or scaffold 01757 of the Cameor v1a reference genome.

18. (canceled)

19. The method of claim 18, wherein said high-protein QTL comprises a SNP marker associated with high protein content.

20. The method of claim 19, wherein the SNP marker is selected from the group consisting of Ps03_531239107, Ps05_49389403, Ps01_20222535, Ps01_22514126, Ps01_55991509, Ps01_78756169, Ps01_87632539, Ps01_88206114, Ps01_95579585, Ps01_113369982, Ps01_113369984, Ps01_120406755, Ps01_160458316, Ps01_264925535, Ps01_279286967, Ps01_280789385, Ps01_300614888, Ps01_324252121, Ps01_349096914, Ps01_367706416, Ps01_380906255, Ps01_436651445, Ps01_440892085, Ps02_17543117, Ps02_64161520, Ps02_149117835, Ps02_162193050, Ps02_249953551, Ps02_282186543, Ps02_293278647, Ps02_296256342, Ps02_298578096, Ps02_313767424, Ps02_389051201, Ps03_238101773, Ps03_241025997, Ps03_481796573, Ps03_483314788, Ps03_507346266, Ps03_511404191, Ps03_513771826, Ps03_531014546, Ps03_531232613, Ps04_9648139, Ps04_26115694, Ps04_106176050, Ps04_119030031, Ps04_126746363, Ps04_133748675, Ps04_140768543, Ps04_196413843, Ps04_198084088, Ps04_198169869, Ps04_256098157, Ps04_263312773, Ps04_284358817, Ps04_327258970, Ps04_347955117, Ps04_374415380, Ps04_378090615, Ps04_386293806, Ps04_434414625, Ps04_445153308, Ps04_457675265, Ps04_463538432, Ps04_464099084, Ps04_467088335, Ps05_5262178, Ps05_17115394, Ps05_23320549, Ps05_48702172, Ps05_51336818, Ps05_53552642, Ps05_54722636, Ps05_134772954, Ps05_139126831, Ps05_173144250, Ps05_175640373, Ps05_217776534, Ps05_265627777, Ps05_268032915, Ps05_277838646, Ps05_284520856, Ps05_289702502, Ps05_290623435, Ps05_320108884, Ps05_337541850, Ps05_338232797, Ps05_352490739, Ps05_358014672, Ps05_409869744, Ps05_456631333, Ps05_500234888, Ps05_534247077, Ps05_543517276, Ps05_550603121, Ps05_551582581, Ps05_556990553, Ps05_564305756, Ps05_568744565, Ps05_576520275, Ps05_591946858, Ps05_596172019, Ps06_1859845, Ps06_32259152, Ps06_71058460, Ps06_75832558, Ps06_79052113, Ps06_91302660, Ps06_97595572, Ps06_108179595, Ps06_137271101, Ps06_261243645, Ps06_375201129, Ps06_383667570, Ps06_402503684, Ps06_410567663, Ps06_427519500, Ps06_446483044, Ps07_9801763, Ps07_20773355, Ps07_46743665, Ps07_50335973, Ps07_55350864, Ps07_57031312, Ps07_58281807, Ps07_84885129, Ps07_89781713, Ps07_112377551, Ps07_131261098, Ps07_155895151, Ps07_173321635, Ps07_231299734, Ps07_235684752, Ps07_238767894, Ps07_241735133, Ps07_274069066, Ps07_314769485, Ps07_327087818, Ps07_337883272, Ps07_466233654, Ps07_466821729, and Ps07_482615897.

21. The method of claim 19-or 20, wherein the SNP marker is in the Pisum sativum genome and corresponds to: a C at position 425968088 of chromosome 3; a C at position 563531992 of chromosome 5; a C at position 101 of SEQ ID NO: 47 in a genomic region comprising the nucleic acid sequence of SEQ ID NO: 47, or at a corresponding position of a genomic region at least 50 nucleotides of which is aligned to SEQ ID NO: 47 for at least 90% sequence identity; an A at position 36835261 of chromosome 5; an A at position 101 of SEQ ID NO: 148 in a genomic region comprising the nucleic acid sequence of SEQ ID NO: 148, or at a corresponding position of a genomic region at least 50 nucleotides of which is aligned to SEQ ID NO: 148 for at least 90% sequence identity; a G at position 36095 of scaffold 04655; a T at position 108299170 of chromosome 1LG6; a G at position 157869 of scaffold 00066; a T at position 23108565 of chromosome 1LG6; an A at position 122338117 of chromosome 1LG6; a G at position 306857129 of chromosome 1LG6; a G at position 45686305 of chromosome 1LG6; an A at position 371072249 of chromosome 1LG6; a G at position 72083191 of chromosome 1LG6; a C at position 79225752 of chromosome 1LG6; a T at position 8290 of scaffold 02021; an A at position 119392808 of chromosome 2LG1; a T at position 10842575 of chromosome 2LG1; an A at position 169314375 of chromosome 2LG1; a G at position 286755665 of chromosome 2LG1; a G at position 91532 of scaffold 00644; a C at position 301660243 of chromosome 2LG1; an A at position 420361771 of chromosome 2LG1; a G at position 426699364 of chromosome 2LG1; a C at position 26206979 of chromosome 2LG1; a G at position 30219372 of chromosome 3LG5; an A at position 393751811 of chromosome 3LG5; an A at position 417958980 of chromosome 3LG5; a G at position 421049387 of chromosome 3LG5; a T at position 83578489 of chromosome 4LG4; a C at position 109277412 of chromosome 4LG4; an A at position 117043426 of chromosome 4LG4; a G at position 163719335 of chromosome 4LG4; a T at position 18486554 of chromosome 4LG4; a C at position 247901046 of chromosome 4LG4; a T at position 2191851 of chromosome 4LG4; a C at position 444278355 of chromosome 4LG4; a T at position 445125850 of chromosome 4LG4; a T at position 5972665 of chromosome 4LG4; an A at position 96025751 of chromosome 5LG3; an A at position 12104 of scaffold 00462; a C at position 178039871 of chromosome 5LG3; an A at position 132883215 of chromosome 5LG3; a G at position 24130766 of chromosome 5LG3; a T at position 228797264 of chromosome 5LG3; a G at position 239060496 of chromosome 5LG3; a C at position 2288318 of chromosome 5LG3; a G at position 331834371 of chromosome 5LG3; a T at position 50774 of scaffold 02833; a C at position 37349400 of chromosome 5LG3; a G at position 39703 of super-scaffold 888; a G at position 509926370 of chromosome 5LG3; a C at position 509729669 of chromosome 5; an A at position 522716439 of chromosome 5LG3; a T at position 124873928 of chromosome 5LG3; a G at position 551226342 of chromosome 5LG3; an A at position 547326524 of chromosome 5; a T at position 1621846 of chromosome 6LG2; an A at position 4002 of scaffold 00839; a T at position 374758162 of chromosome 6LG2; a T at position 401325650 of chromosome 6LG2; a C at position 426328393 of chromosome 6LG2; a G at position 438943398 of chromosome 6; a T at position 72341 of scaffold 02959; a G at position 89032441 of chromosome 7LG7; an A at position 19382049 of chromosome 7LG7; a C at position 310437720 of chromosome 7LG7; a T at position 310515874 of chromosome 7LG7; a C at position 335690162 of chromosome 7LG7; an A at position 322450055 of chromosome 7; a G at position 10989 of scaffold 00840; an A at position 460750292 of chromosome 7LG7; a G at position 13304 of scaffold 06512; a T at position 52311972 of chromosome 7; a G at position 50802012 of chromosome 7LG7; an A at position 56383957 of chromosome 7LG7; a G at position 1311773 of chromosome 7LG7; a G at position 8316015 of chromosome 7LG7; a C at position 36097 of scaffold 04655; an A at position 20877277 of chromosome 1LG6; a T at position 194893633 of chromosome 1LG6; a G at position 72152 of scaffold 03789; an A at position 30542636 of chromosome 1LG6; a T at position 116776833 of chromosome 1LG6; an A at position 288453266 of chromosome 1LG6; a G at position 367968951 of chromosome 1LG6; a T at position 51330566 of chromosome 1LG6; a C at position 29379 of scaffold 02116; a G at position 87185358 of chromosome 1LG6; a G at position 5787797 of chromosome 2LG1; a C at position 87090294 of chromosome 2LG1; an A at position 383268619 of chromosome 2LG1; a C at position 104891818 of chromosome 3LG5; a G at position 72342 of scaffold 00254; a T at position 173063548 of chromosome 3LG5; a T at position 174636272 of chromosome 3LG5; a C at position 396332351 of chromosome 3LG5; a G at position 423551062 of chromosome 3LG5; a C at position 827484 of chromosome 3LG5; an A at position 425962517 of chromosome 3; a T at position 94928513 of chromosome 4LG4; an A at position 47049 of scaffold 02127; a C at position 165487268 of chromosome 4LG4; an A at position 165597701 of chromosome 4LG4; an A at position 218884465 of chromosome 4LG4; a G at position 228522691 of chromosome 4LG4; a C at position 352524054 of chromosome 4LG4; an A at position 363782042 of chromosome 4LG4; a G at position 285377934 of chromosome 4LG4; a G at position 389689436 of chromosome 4LG4; an A at position 145804 of scaffold 00706; an A at position 374997960 of chromosome 4LG4; a G at position 418184353 of chromosome 4LG4; an A at position 420833970 of chromosome 4LG4; an A at position 134409547 of chromosome 5LG3; an A at position 20543180 of chromosome 5LG3; a G at position 217510948 of chromosome 5LG3; a C at position 84199 of scaffold 02449; a G at position 234213508 of chromosome 5LG3; a T at position 236504935 of chromosome 5LG3; a C at position 268153046 of chromosome 5LG3; a T at position 278088295 of chromosome 5LG3; an A at position 84459019 of chromosome 5LG3; a G at position 306598116 of chromosome 5LG3; a G at position 413429268 of chromosome 5; a G at position 35018191 of chromosome 5LG3; a C at position 362278 of chromosome 5LG3; a C at position 492763269 of chromosome 5LG3; a G at position 499899891 of chromosome 5LG3; a T at position 39908055 of chromosome 5LG3; a T at position 143390359 of chromosome 5LG3; a G at position 535824046 of chromosome 5LG3; an A at position 89382481 of chromosome 6LG2; a T at position 111705293 of chromosome 6LG2; an A at position 191916418 of chromosome 6LG2; a G at position 303558968 of chromosome 6LG2; a T at position 406586297 of chromosome 6LG2; a C at position 17855 of scaffold 05469; an A at position 62010766 of chromosome 6LG2; a T at position 64688438 of chromosome 6LG2; a C at position 75309486 of chromosome 6LG2; a T at position 86301013 of chromosome 6LG2; an A at position 45871271 of chromosome 7LG7; an A at position 16557044 of chromosome 7LG7; a T at position 223304507 of chromosome 7LG7; an A at position 158981077 of chromosome 7LG7; a C at position 365136400 of chromosome 7LG7; a G at position 47207 of scaffold 01757; an A at position 481276628 of chromosome 7LG7; an A at position 52153701 of chromosome 7LG7; or a G at position 88161594 of chromosome 7LG7 of the Cameor v1a reference genome.

22. A high-protein population of pea plants or seeds produced by the method of claim 17, wherein said high-protein population has a greater frequency of the one or more high-protein alleles than said first population of pea plants.

23. (canceled)

24. The high-protein population of pea plants or seeds of claim 22 or 23, comprising at least 20%, 21%, 22%, 23%, 24%, 25%, 26%, or 27% protein by weight or comprising protein content that is greater by at least 1.0% dry weight relative to pea plants or seeds without said one or more high-protein alleles.

25. (canceled)

26. The high-protein population of pea plants or seeds of claim 22, wherein the pea plants or seeds comprising said one or more high-protein alleles have yield that is 99% or greater relative to pea plants or seeds without said one or more high-protein alleles.

27. A nucleic acid molecule for detecting a high-protein molecular marker in a Pisum sativum genome, wherein the nucleic acid molecule comprises at least 15 nucleotides that include or are immediately adjacent to the marker, wherein the nucleic acid molecule is at least 90 percent identical to a sequence of the same number of consecutive nucleotides in either strand of DNA that include or are immediately adjacent to the marker.

28. The nucleic acid molecule of claim 27, wherein the high-protein molecular marker is a SNP marker, and wherein the SNP marker is in a Pisum sativum genome and corresponds to a C at position 425968088 of chromosome 3; a C at position 563531992 of chromosome 5; a C at position 101 of SEQ ID NO: 47 in a genomic region comprising the nucleic acid sequence of SEQ ID NO: 47, or at a corresponding position of a genomic region at least 50 nucleotides of which is aligned to SEQ ID NO: 47 for at least 90% sequence identity; an A at position 36835261 of chromosome 5; an A at position 101 of SEQ ID NO: 148 in a genomic region comprising the nucleic acid sequence of SEQ ID NO: 148, or at a corresponding position of a genomic region at least 50 nucleotides of which is aligned to SEQ ID NO: 148 for at least 90% sequence identity; a G at position 36095 of scaffold 04655; a T at position 108299170 of chromosome 1LG6; a G at position 157869 of scaffold 00066; a T at position 23108565 of chromosome 1LG6; an A at position 122338117 of chromosome 1LG6; a G at position 306857129 of chromosome 1LG6; a G at position 45686305 of chromosome 1LG6; an A at position 371072249 of chromosome 1LG6; a G at position 72083191 of chromosome 1LG6; a C at position 79225752 of chromosome 1LG6; a T at position 8290 of scaffold 02021; an A at position 119392808 of chromosome 2LG1; a T at position 10842575 of chromosome 2LG1; an A at position 169314375 of chromosome 2LG1; a G at position 286755665 of chromosome 2LG1; a G at position 91532 of scaffold 00644; a C at position 301660243 of chromosome 2LG1; an A at position 420361771 of chromosome 2LG1; a G at position 426699364 of chromosome 2LG1; a C at position 26206979 of chromosome 2LG1; a G at position 30219372 of chromosome 3LG5; an A at position 393751811 of chromosome 3LG5; an A at position 417958980 of chromosome 3LG5; a G at position 421049387 of chromosome 3LG5; a T at position 83578489 of chromosome 4LG4; a C at position 109277412 of chromosome 4LG4; an A at position 117043426 of chromosome 4LG4; a G at position 163719335 of chromosome 4LG4; a T at position 18486554 of chromosome 4LG4; a C at position 247901046 of chromosome 4LG4; a T at position 2191851 of chromosome 4LG4; a C at position 444278355 of chromosome 4LG4; a T at position 445125850 of chromosome 4LG4; a T at position 5972665 of chromosome 4LG4; an A at position 96025751 of chromosome 5LG3; an A at position 12104 of scaffold 00462; a C at position 178039871 of chromosome 5LG3; an A at position 132883215 of chromosome 5LG3; a G at position 24130766 of chromosome 5LG3; a T at position 228797264 of chromosome 5LG3; a G at position 239060496 of chromosome 5LG3; a C at position 2288318 of chromosome 5LG3; a G at position 331834371 of chromosome 5LG3; a T at position 50774 of scaffold 02833; a C at position 37349400 of chromosome 5LG3; a G at position 39703 of super-scaffold 888; a G at position 509926370 of chromosome 5LG3; a C at position 509729669 of chromosome 5; an A at position 522716439 of chromosome 5LG3; a T at position 124873928 of chromosome 5LG3; a G at position 551226342 of chromosome 5LG3; an A at position 547326524 of chromosome 5; a T at position 1621846 of chromosome 6LG2; an A at position 4002 of scaffold 00839; a T at position 374758162 of chromosome 6LG2; a T at position 401325650 of chromosome 6LG2; a C at position 426328393 of chromosome 6LG2; a G at position 438943398 of chromosome 6; a T at position 72341 of scaffold 02959; a G at position 89032441 of chromosome 7LG7; an A at position 19382049 of chromosome 7LG7; a C at position 310437720 of chromosome 7LG7; a T at position 310515874 of chromosome 7LG7; a C at position 335690162 of chromosome 7LG7; an A at position 322450055 of chromosome 7; a G at position 10989 of scaffold 00840; an A at position 460750292 of chromosome 7LG7; a G at position 13304 of scaffold 06512; a T at position 52311972 of chromosome 7; a G at position 50802012 of chromosome 7LG7; an A at position 56383957 of chromosome 7LG7; a G at position 1311773 of chromosome 7LG7; a G at position 8316015 of chromosome 7LG7; a C at position 36097 of scaffold 04655; an A at position 20877277 of chromosome 1LG6; a T at position 194893633 of chromosome 1LG6; a G at position 72152 of scaffold 03789; an A at position 30542636 of chromosome 1LG6; a T at position 116776833 of chromosome 1LG6; an A at position 288453266 of chromosome 1LG6; a G at position 367968951 of chromosome 1LG6; a T at position 51330566 of chromosome 1LG6; a C at position 29379 of scaffold 02116; a G at position 87185358 of chromosome 1LG6; a G at position 5787797 of chromosome 2LG1; a C at position 87090294 of chromosome 2LG1; an A at position 383268619 of chromosome 2LG1; a C at position 104891818 of chromosome 3LG5; a G at position 72342 of scaffold 00254; a T at position 173063548 of chromosome 3LG5; a T at position 174636272 of chromosome 3LG5; a C at position 396332351 of chromosome 3LG5; a G at position 423551062 of chromosome 3LG5; a C at position 827484 of chromosome 3LG5; an A at position 425962517 of chromosome 3; a T at position 94928513 of chromosome 4LG4; an A at position 47049 of scaffold 02127; a C at position 165487268 of chromosome 4LG4; an A at position 165597701 of chromosome 4LG4; an A at position 218884465 of chromosome 4LG4; a G at position 228522691 of chromosome 4LG4; a C at position 352524054 of chromosome 4LG4; an A at position 363782042 of chromosome 4LG4; a G at position 285377934 of chromosome 4LG4; a G at position 389689436 of chromosome 4LG4; an A at position 145804 of scaffold 00706; an A at position 374997960 of chromosome 4LG4; a G at position 418184353 of chromosome 4LG4; an A at position 420833970 of chromosome 4LG4; an A at position 134409547 of chromosome 5LG3; an A at position 20543180 of chromosome 5LG3; a G at position 217510948 of chromosome 5LG3; a C at position 84199 of scaffold 02449; a G at position 234213508 of chromosome 5LG3; a T at position 236504935 of chromosome 5LG3; a C at position 268153046 of chromosome 5LG3; a T at position 278088295 of chromosome 5LG3; an A at position 84459019 of chromosome 5LG3; a G at position 306598116 of chromosome 5LG3; a G at position 413429268 of chromosome 5; a G at position 35018191 of chromosome 5LG3; a C at position 362278 of chromosome 5LG3; a C at position 492763269 of chromosome 5LG3; a G at position 499899891 of chromosome 5LG3; a T at position 39908055 of chromosome 5LG3; a T at position 143390359 of chromosome 5LG3; a G at position 535824046 of chromosome 5LG3; an A at position 89382481 of chromosome 6LG2; a T at position 111705293 of chromosome 6LG2; an A at position 191916418 of chromosome 6LG2; a G at position 303558968 of chromosome 6LG2; a T at position 406586297 of chromosome 6LG2; a C at position 17855 of scaffold 05469; an A at position 62010766 of chromosome 6LG2; a T at position 64688438 of chromosome 6LG2; a C at position 75309486 of chromosome 6LG2; a T at position 86301013 of chromosome 6LG2; an A at position 45871271 of chromosome 7LG7; an A at position 16557044 of chromosome 7LG7; a T at position 223304507 of chromosome 7LG7; an A at position 158981077 of chromosome 7LG7; a C at position 365136400 of chromosome 7LG7; a G at position 47207 of scaffold 01757; an A at position 481276628 of chromosome 7LG7; an A at position 52153701 of chromosome 7LG7; and a G at position 88161594 of chromosome 7LG7 of the Cameor v1a reference genome.

29. (canceled)

30. The nucleic acid molecule of claim 27, further comprising a detectable label.

31. (canceled)
